SEQUENCE LISTING (55 sequences)

SEQ ID NO: 1 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 3 - protein, 256 aa, Homo sapiens
MYRMQLLSCIALSLALVTNSMPRGPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 266 aa, Homo sapiens
METDTLLLWVLLLWVPGSTGDAAQPARRAVRSLVPSSDPRKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 318 aa, Homo sapiens
METDTLLLWVLLLWVPGSTGDAAQPARRAVRSLVPSSDPELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 6 - protein, 268 aa, Homo sapiens
METDTLLLWVLLLWVPGSTGDAAQPARRAVRSLVPSSDPESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 7 - protein, 252 aa, Mus musculus
MYRMQLLSCIALSLALVTNSMPRGPVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 253 aa, Mus musculus
MYRMQLLSCIALSLALVTNSMPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 9 - protein, 259 aa, Mus musculus
MYRMQLLSCIALSLALVTNSEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 10 - protein, 253 aa, Mus musculus
MYRMQLLSCIALSLALVTNSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 11 - protein, 619 aa, synthetic construct
  REGION 1..619: note = human IgG Fc - B4GALT1 fusion protein
MYRMQLLSCIALSLALVTNSMPRGPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGAPDLKLMGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 12 - protein, 627 aa, synthetic construct
  REGION 1..627: note = human IgG1 Fc - ST6GAL1 fusion protein
MYRMQLLSCIALSLALVTNSMPRGPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGAPDLKLMEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 13 - protein, 611 aa, synthetic construct
  REGION 1..611: note = mouse IgG1 Fc - B4GALT1 fusion protein
MYRMQLLSCIALSLALVTNSMVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGAPDLKLMGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 14 - protein, 623 aa, synthetic construct
  REGION 1..623: note = mouse IgG1 Fc - ST6GAL1 fusion protein
MYRMQLLSCIALSLALVTNSMPRGPVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGAPDLKLMEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 15 - protein, 600 aa, synthetic construct
  REGION 1..600: note = canine IgG-A (IgG1) Fc - ST6GAL1 fusion protein
MYRMQLLSCIALSLALVTNSPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGKEFQMVRGLEKQAATLSSTQNPPRASQALGSPRGPVKAKSEASFQVWNKDSSSKNLIPRLQKIWRNYLNMNKYKVSYKGPGPGVKFSAEALHCHLRDHVNVSMVEATDFPFNTSEWEGFLPKENIRTKAGPWGRCAVVSSAGSLKSSQLGREIDDHDAVLRFNGAPTASFQQDVGTKTTIRLMNSQLVTTEGRFLKDSLYNEGILIVWDPSVYHSDIPKWYQSPDYSFFENYKSYRKLHPDQPFYILKPQMPWELWDIIQEVSPEEIQPNPPSSGMLGIIIMMTLCDQVDIYEFLPSKRKTDVCYYYQKFFDSACTMGAYHPLLFEKNLVKHLNQGTDEDIYLLGKATLPGFRRIRC

SEQ ID NO: 16 - protein, 502 aa, synthetic construct
  REGION 1..502: note = canine IgG-A(IgG1) Fc - B4GALT1 fusion protein
MYRMQLLSCIALSLALVTNSPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGKMVIEFNMPVDLKLVEKQNPEVKVGGRYTPKNCISPHKVAIIIPFRNRQEHLKYWLYYLHPILQRQQLDYGIYVINQAGETMFNRAKLLNIGFQEALKDYDYNCFVFSDVDLIPMNDHNAYRCFSQPRHISVAMDKFGFSLPYVQYFGGVSALSKEQFLTINGFPNNYWGWGGEDDDIYNRLVFKGMSVSRPNAMVGKCRMIRHSRDKKNEPNPQRFDRIAHTKETMLSDGLNTLTYKVLDKERNPLYTKITVDIGTPS

SEQ ID NO: 17 - protein, 599 aa, synthetic construct
  REGION 1..599: note = feline IgG1 Fc-ST6GAL1 fusion protein
MYRMQLLSCIALSLALVTNSPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGKDFQVLRGLEKQAETSSSTQDPHRGSQALSSPRGPAKAKPEASFQVWNKDSSSKNLIPRLQKIWRNYLNMNKYKVSYKGPGPGVKLSAEALHCHLRERVNVSMVEVTDFPFNTSEWEGFLPKENIRTKAGPWGTCAVVSSAGSLKSSQLGREIDDHDAVLRFNGAPTANFQQDVGTKTTIRLMNSQLVTTEGRFLKDSLYNEGILIVWDPSVYHSDIPKWYQSPDYSFFENYKSYRKLHPDQPFYILRPQMPWELWDIIQEVSPEEIQPNPPSSGMLGIIIMMTLCDQVDIYEFLPSKRKTDVCYYYQKFFDSACTMGAYHPLLFEKNLVKHLNQGTDEDIYLLGKATLPGFRRIRC

SEQ ID NO: 18 - protein, 603 aa, synthetic construct
  REGION 1..603: note = feline IgG1 Fc - B4GALT1 fusion protein
MYRMQLLSCIALSLALVTNSPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGKYLAGRDLNRLPQLVGVPTPLQGGSNGAAAIEQPSAELRPRGAPPLPLLDASSELRSGRDSSPDADSHPGPGPASNLTSAPVPSTTVLSLLACPEESPLLVGPMVIEFNMPVDLKLVEKQNPEVKVGGRYTPKNCISPHKVAIIIPFRNRQEHLKYWLYYLHPILQRQQLDYGIYVINQAGETMFNRAKLLNIGFQEALKDYDYNCFVFSDVDLIPMNDHNAYRCFSQPRHISVAMDKFGFSLPYVQYFGGVSALSKQQFLTINGFPNNYWGWGGEDDDIFNRLVFRGMSVSRPNAVVGKCRMIRHSRDKKNEPNPQRFDRIAHTKETMLSDGLNTLSYKVLDIERNPLYTKITVDIGTPS

SEQ ID NO: 19 - protein, 468 aa, Canis familiaris
MESVFCWVFLVVILKGVQGEVQLVESGGDLVKPGGSLRLSCVASGFTFSSYYMHWIRQAPGKGLQRVAHIRGDGRTTHYADAMKGRFTISRDNAKNTLYLQMNSLTVEDTAIYYCVKDIYYGVGDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 20 - protein, 473 aa, Canis familiaris
MESVLFWVFLVTILKGVQGEVRLVESGGTLVKPGGSLKLSCVASGFTFRRYSMDWVRQAPGKSLQWVAGINGDGTGTSYSQTVKGRFTISRDNAKNTLYLQINSLRAEDSAVYYCAKSWSRNGDLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 21 - protein, 474 aa, Canis familiaris
MESVLYWVFLVAILKGVQGDVQLVESGGDLVKPGGSLRLSCVASGFTFSSCAMSWVRQSPGKGPQWVATIRYDGSDIYYADAVKGRFSISRDNAKNTVYLQMNSLRAEDTAVYYCAKAPPYDSYHYGMDYWGPGTSLFVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 22 - protein, 470 aa, Canis familiaris
MESVLCWVFLVSILKGVQGEVQLVESGGDLVKPGGSLRLSCVASGFTFSDYGMSWVRQSPGKGLQWVAAVSNRGDTYYADAVKGRFTISRDNAKNTLYLQMSSLKAEDTAIYHCVTGVWPRHYYGMDHWGNGTSLFVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 23 - protein, 399 aa, Canis familiaris
MIHTNLKKKFSCCVLAFLLFAVICVWKEKKKGSYYDSLKLQTKEFQMVRGLEKQAATLSSTQNPPRASQALGSPRGPVKAKSEASFQVWNKDSSSKNLIPRLQKIWRNYLNMNKYKVSYKGPGPGVKFSAEALHCHLRDHVNVSMVEATDFPFNTSEWEGFLPKENIRTKAGPWGRCAVVSSAGSLKSSQLGREIDDHDAVLRFNGAPTASFQQDVGTKTTIRLMNSQLVTTEGRFLKDSLYNEGILIVWDPSVYHSDIPKWYQSPDYSFFENYKSYRKLHPDQPFYILKPQMPWELWDIIQEVSPEEIQPNPPSSGMLGIIIMMTLCDQVDIYEFLPSKRKTDVCYYYQKFFDSACTMGAYHPLLFEKNLVKHLNQGTDEDIYLLGKATLPGFRRIRC

SEQ ID NO: 24 - protein, 258 aa, Canis familiaris
MVIEFNMPVDLKLVEKQNPEVKVGGRYTPKNCISPHKVAIIIPFRNRQEHLKYWLYYLHPILQRQQLDYGIYVINQAGETMFNRAKLLNIGFQEALKDYDYNCFVFSDVDLIPMNDHNAYRCFSQPRHISVAMDKFGFSLPYVQYFGGVSALSKEQFLTINGFPNNYWGWGGEDDDIYNRLVFKGMSVSRPNAMVGKCRMIRHSRDKKNEPNPQRFDRIAHTKETMLSDGLNTLTYKVLDKERNPLYTKITVDIGTPS

SEQ ID NO: 25 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 26 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 27 - protein, 399 aa, Felis catus
MIHANLKKKFSCCVLAFLLFAIICVWKEKKKGTYYDSLKLQSKDFQVLRGLEKQAETSSSTQDPHRGSQALSSPRGPAKAKPEASFQVWNKDSSSKNLIPRLQKIWRNYLNMNKYKVSYKGPGPGVKLSAEALHCHLRERVNVSMVEVTDFPFNTSEWEGFLPKENIRTKAGPWGTCAVVSSAGSLKSSQLGREIDDHDAVLRFNGAPTANFQQDVGTKTTIRLMNSQLVTTEGRFLKDSLYNEGILIVWDPSVYHSDIPKWYQSPDYSFFENYKSYRKLHPDQPFYILRPQMPWELWDIIQEVSPEEIQPNPPSSGMLGIIIMMTLCDQVDIYEFLPSKRKTDVCYYYQKFFDSACTMGAYHPLLFEKNLVKHLNQGTDEDIYLLGKATLPGFRRIRC

SEQ ID NO: 28 - protein, 373 aa, Felis catus
AVCALHLGVTLVYYLAGRDLNRLPQLVGVPTPLQGGSNGAAAIEQPSAELRPRGAPPLPLLDASSELRSGRDSSPDADSHPGPGPASNLTSAPVPSTTVLSLLACPEESPLLVGPMVIEFNMPVDLKLVEKQNPEVKVGGRYTPKNCISPHKVAIIIPFRNRQEHLKYWLYYLHPILQRQQLDYGIYVINQAGETMFNRAKLLNIGFQEALKDYDYNCFVFSDVDLIPMNDHNAYRCFSQPRHISVAMDKFGFSLPYVQYFGGVSALSKQQFLTINGFPNNYWGWGGEDDDIFNRLVFRGMSVSRPNAVVGKCRMIRHSRDKKNEPNPQRFDRIAHTKETMLSDGLNTLSYKVLDIERNPLYTKITVDIGTPS

SEQ ID NO: 29 - protein, 990 aa, Bos taurus
GCCTCCACCACAGCCCCGAAAGTCTACCCTCTGAGTTCTTGCTGCGGGGACAAGTCCAGCTCCACCGTGACCCTGGGCTGCCTGGTCTCCAGCTACATGCCCGAGCCGGTGACCGTGACCTGGAACTCGGGTGCCCTGAAGAGCGGCGTGCACACCTTCCCGGCCGTCCTTCAGTCCTCCGGGCTCTACTCTCTCAGCAGCATGGTGACCGTGCCCGGCAGCACCTCAGGAACCCAGACCTTCACCTGCAACGTAGCCCACCCGGCCAGCAGCACCAAGGTGGACAAGGCTGTTGATCCCAGATGCAAAACAACCTGTGACTGTTGCCCACCGCCTGAGCTCCCTGGAGGACCCTCTGTCTTCATCTTCCCACCGAAACCCAAGGACACCCTCACAATCTCGGGAACGCCCGAGGTCACGTGTGTGGTGGTGGACGTGGGCCACGATGACCCCGAGGTGAAGTTCTCCTGGTTCGTGGACGACGTGGAGGTAAACACAGCCACGACGAAGCCGAGAGAGGAGCAGTTCAACAGCACCTACCGCGTGGTCAGCGCCCTGCGCATCCAGCACCAGGACTGGACTGGAGGAAAGGAGTTCAAGTGCAAGGTCCACAACGAAGGCCTCCCAGCCCCCATCGTGAGGACCATCTCCAGGACCAAAGGGCCGGCCCGGGAGCCGCAGGTGTATGTCCTGGCCCCACCCCAGGAAGAGCTCAGCAAAAGCACGGTCAGCCTCACCTGCATGGTCACCAGCTTCTACCCAGACTACATCGCCGTGGAGTGGCAGAGAAATGGGCAGCCTGAGTCAGAGGACAAGTACGGCACGACCCCTCCCCAGCTGGACGCCGACGGCTCCTACTTCCTGTACAGCAGGCTCAGGGTGGACAGGAACAGCTGGCAGGAAGGAGACACCTACACGTGTGTGGTGATGCACGAGGCCCTGCACAATCACTACACGCAGAAGTCCACCTCTAAGTCTGCGGGTAAATGA

SEQ ID NO: 30 - protein, 981 aa, Bos taurus
GCCTCCACCACAGCCCCGAAAGTCTACCCTCTGGCATCCAGCTGCGGAGACACATCCAGCTCCACCGTGACCCTGGGCTGCCTGGTGTCCAGCTACATGCCCGAGCCGGTGACCGTGACCTGGAACTCGGGTGCCCTGAAGAGCGGCGTGCACACCTTCCCGGCTGTCCTTCAGTCCTCCGGGCTCTACTCTCTCAGCAGCATGGTGACCGTGCCCGCCAGCAGCTCAGGACAGACCTTCACCTGCAACGTAGCCCACCCGGCCAGCAGCACCAAGGTGGACAAGGCTGTTGGGGTCTCCATTGACTGCTCCAAGTGTCATAACCAGCCTTGCGTGAGGGAACCATCTGTCTTCATCTTCCCACCGAAACCCAAAGACACCCTGATGATCACAGGAACGCCCGAGGTCACGTGTGTGGTGGTGAACGTGGGCCACGATAACCCCGAGGTGCAGTTCTCCTGGTTCGTGGATGACGTGGAGGTGCACACGGCCAGGTCGAAGCCAAGAGAGGAGCAGTTCAACAGCACGTACCGCGTGGTCAGCGCCCTGCCCATCCAGCACCAGGACTGGACTGGAGGAAAGGAGTTCAAGTGCAAGGTCAACAACAAAGGCCTCTCGGCCCCCATCGTGAGGATCATCTCCAGGAGCAAAGGGCCGGCCCGGGAGCCGCAGGTGTATGTCCTGGACCCACCCAAGGAAGAGCTCAGCAAAAGCACGCTCAGCGTCACCTGCATGGTCACCGGCTTCTACCCAGAAGATGTAGCCGTGGAGTGGCAGAGAAACCGGCAGACTGAGTCGGAGGACAAGTACCGCACGACCCCGCCCCAGCTGGACACCGACCGCTCCTACTTCCTGTACAGCAAGCTCAGGGTGGACAGGAACAGCTGGCAGGAAGGAGACGCCTACACGTGTGTGGTGATGCACGAGGCCCTGCACAATCACTACATGCAGAAGTCCACCTCTAAGTCTGCGGGTAAATGA

SEQ ID NO: 31 - protein, 1764 aa, Bos taurus
CCACCATGCCGGCCGGTCATCAGACCCTGGAAGCAGGCAGTGGCTGGGCTTGGAAGTGCCCCAGGCCTGGGCTCCTGAGGTCCTGCTGGACCCAGCATTCACCCAGCCTCCTCTCTCACAGCCTCCACCACAGCCCCGAAAGTCTACCCTCTGGCATCCAGCTGCGGAGACACATCCAGCTCCACCGTGACCCTGGGCTGCCTGGTCTCCAGCTACATGCCCGAGCCGGTGACCGTGACCTGGAACTCGGGTGCCCTGAAGAGCGGCGTGCACACCTTCCCGGCCGTCCGGCAGTCCTCTGGGCTGTACTCTCTCAGCAGCATGGTGACTGTGCCCGCCAGCAGCTCAGAAACCCAGACCTTCACCTGCAACGTAGCCCACCCGGCCAGCAGCACCAAGGTGGACAAGGCTGTCACTGCAAGGCGTCCAGTCCCGACGACGCCAAAGACAACTATCCCTCCTGGAAAACCCACAACCCCAAGTATGTGAATCCCACACTCCTGCCTGCAGGGGCCTCAGCCCAGGGGTGCTGTGAACCAGGCCCAGCGTGTCAGGGGAGGCCCTGTCTGTCTCTCTCTCCTGAAGGTCTCACAGGCTTGGGGAGGGGTGTTGGACTTTCCACGATGTCCAGGCTGCTGCAGGCTGGATGACGCCTCGGCCCCTGGCCCCACAGAGGCGGGCCCTCGGCTCGGACTACCAAAACTTGTCCCTGCCCTAAGCCCAGACCACAGCTTCCTGCCCCTGGTAACCCCCGGTCTGCTCTCTCTGCAGAGTCTGAAGTTGAAAAGACACCCTGCCAGTGTTCCAAATGCCCAGGTAAGTCAGCTGGCTTCATCCTCTGTCTGACACTGGCGAACAGCACTCAGGGCAGCCGGGTGGAGGACGCGGGTCCAAAGGAGGTTTCCCAGGTGCAGAACCCCCACCATGCTTTCTCACCAACCACAGAACCTCTGGGAGGACTGTCTGTCTTCATCTTCCCACCGAAACCCAAGGACACCCTCACAATCTCGGGAACGCCCGAGGTCACGTGTGTGGTGGTGGACGTGGGCCAGGATGACCCCGAGGTGCAGTTCTCCTGGTTCGTGGACGACGTGGAGGTGCACACGGCCAGGACGAAGCCGAGAGAGGAGCAGTTCAACAGCACCTACCGCGTGGTCAGCGCCCTGCGCATCCAGCACCAGGACTGGCTGCAGGGAAAGGAGTTCAAGTGCAAGGTCAACAACAAAGGCCTCCCGGCCCCCATTGTGAGGACCATCTCCAGGACCAAAGGTGGGCCAGGTGGACTGGACCGGGAGGGTCCCGTGGGCCAATCAGAGTGACCGCTGTACGGGACCGGGCCCTGTGGGCCAATCAGAGTGACCGCTGGACGGGACCGGGAGGGTCCCGTGGGCCAATCAGAGTGACCGCTGTGCTAACAGCCTTCCTGTCCCCACAGGGCAGGCCCGGGAGCCGCAGGTGTATGTCCTGGCCCCACCCCGGGAAGAGCTCAGCAAAAGCACGCTCAGCCTCACCTGCCTGATCACCGGTTTCTACCCAGAAGAGATAGACGTGGAGTGGCAGAGAAATGGGCAGCCTGAGTCGGAGGACAAGTACCACACGACCGCACCCCAGCTGGATGCTGACGGCTCCTACTTCCTGTACAGCAAGCTCAGGGTGAACAAGAGCAGCTGGCAGGAAGGAGACCACTACACGTGTGCAGTGATGCACGAAGCTTTACGGAATCACTACAAAGAGAAGTCCATCTCGAGGTCTCCGGGTAAATGA

SEQ ID NO: 32 - protein, 405 aa, Bos taurus
MTRTSLKKKVFSCCVLIFLLFAIICVWKEKKKGNYYEFLKLQNKEYQVLQGLEKLAVSSSSQPVSSSSTHNPQRNIQALGGPKAKLKATFQVWDKDSSSKNLAPRLQTIRKNYLNMNKYKVTYKGPGPGVKFSAEALLCHLRDHVNISMIEATDFPFNTSDWEGYLPQEDIRTKAGPWGRCAVVSSAGSLKSSRLGREIDDHDAVLRFNGAPTVKFQQDVGTKTTIRLVNSQLVTTEAGFLKDSLYNEGILIVWDPSVYHSDIPKWYRNPDYSFFNNFKSYRKLHPDQPFYILKPQMPWELWDIIQEISSELIQPNPPSSGMLGIAIMMSLCDQVDIYEFLPSKRKTDVCYYYQRYFDSACTMGAYHPLLFEKNMVKYLNLGTDEDIYLLGKATLPGFRTIRCGA

SEQ ID NO: 33 - protein, 402 aa, Bos taurus
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQYFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 34 - protein, 337 aa, Equus caballus
ASTTAPKVFALAPGCGTTSDSTVALGCLVSGYFPEPVKVSWNSGSLTSGVHTFPSVLQSSGFYSLSSMVTVPASTWTSETYICNVVHAASNFKVDKRIEPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 35 - protein, 342 aa, Equus caballus
ASTTAPKYFQLTPSCGITSDATVALGCLVSDYYPEPVTVSWNSGALTSGVHTFPSVLQSSGLYALSSMVTVPASTWTSETYICNVAHPASSTKVDKRIPPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 36 - protein, 354 aa, Equus caballus
ASTTAPKVFPLAPSCGTTSDSTVALGCLVSSYFPEPVTVSWNSGTLTSGVRTFPSVLQSSGLYSLSSMVTVPASSLESKTYICNVAHPASSTKVDKRIEPVLPKPTTPAPTVPLTTTVPVETTTPPCPCECPKCPAPELLGGPSVFIFPPKPKDVLMITRTPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFLPTDITVEWQSNEHPEPEGKYRTTEAQKDSDGSYFLYSKLTVETDRWQQGTTFTCVVMHEALHNHVMQKNVSHSPGK

SEQ ID NO: 37 - protein, 335 aa, Equus caballus
  SITE 245: note = misc_feature - Xaa can be any naturally occurring amino acid
  SITE 247: note = misc_feature - Xaa can be any naturally occurring amino acid
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIHLSVLSAVIKECNGGCPAPECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELXRXNVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 38 - protein, 326 aa, Equus caballus
ESPKAPDVFPLTICGNTPDPTVPVGCLVSNYFPEPVTVSWNCDALKGDIHTFPLDLSNSAHHSLSSMMAVPRSSLNQTYICSVAHPASSTKVDKRIVVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 39 - protein, 324 aa, Equus caballus
ASTTAPKVFQLASHSAGTSDSTVALGCLVSSYFPEPVTVSWNSGALTSGVHTFPSVRQSSGLYSLSSMVTVPASSLKSQTYICNVAHPASSTKVDKRIVIKEPCCCPKCPGRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 40 - protein, 406 aa, Equus caballus
MIHSSLKKKFSFCVLVFLLFAVICVWKEKKKGSYYESLKLQTKELQMPRSPEKRAIGSGSKFASSSSTQDPHRNTQGLSNPRSPAKAKPEGSFQVWNKDSSSKNLIPRLQKIWKNYLSMNKYKVSYKGPGPGVKFSADVLRCRLRDEVNVSMVEATDFPFNTSEWEGYLPMEDIRTKAGPWGKCAVVSSAGSLKSSQLGQEIDDHDAVMRFNGAPTASFQQDVGTKTTIRLMNSQLVTTEGRFLKDSLYNEGILIVWDPSVYHSDIPKWYKNPDYSFFDNYKSYRKLHPDQPFYILKPQMPWELWDIIQEISPEEIQPNPPSSGMLGIIIMMTLCDQVDIYEFLPSKRKTDVCYYYQKYFDTACTMGAYHPLLFEKNMVKHLNQGTDEDIYLFGKATLPGFRSIRC

SEQ ID NO: 41 - protein, 266 aa, Equus caballus
SSTSLVGPMMIEFNMAVDLNRVAEENPEVKLGGRYTPKDCISPHKVAIIIPFRNRQEHLKYWLYYLHPILQRQQLDYGIYVINQAGEAMFNRAKLLNVGFQEALKDYDYNCFVFSDVDLIPMNDHNAYRCFSQPRHISVAMDKFGFSLPYVQYFGGVSALSKEQFLTINGFPNNYWGWGGEDDDIFNRLVFKGMSLSRPNAVIGKCRMIRHSRDKKNEPNPQRFDRIAHTKETMFLDGLNTLFYNVLDVQRYPLYTKVTVDIGTPS

SEQ ID NO: 42 - protein, 619 aa, synthetic construct
  REGION 1..619: note = Fc -B4GALT1 fusion protein
MYRMQLLSCIALSLALVTNSMPRGPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGAPDLKLMGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 43 - protein, 627 aa, synthetic construct
  REGION 1..627: note = Fc-ST6GAL1 fusion protein
MYRMQLLSCIALSLALVTNSMPRGPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGAPDLKLMEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 44 - DNA, 39 bp, synthetic construct
  misc_feature 1..39: note = Human ST6GAL1/hST6:EFQ_Fwd primer
  source 1..39: mol_type = other DNA
aaaaagcttatggaattccaggtgttaaagagtctgggg

SEQ ID NO: 45 - DNA, 45 bp, synthetic construct
  misc_feature 1..45: note = Human ST6GAL1/hST6GAL1(K24) primer
  source 1..45: mol_type = other DNA
ggcgcgccaatgaaggaaaagaagaaagggagttactatgattcc

SEQ ID NO: 46 - DNA, 43 bp, synthetic construct
  misc_feature 1..43: note = Human ST6GAL1/hST6GAL1(K27) primer
  source 1..43: mol_type = other DNA
aagcttatgaagaaagggagttactatgattcctttaaattgc

SEQ ID NO: 47 - DNA, 43 bp, synthetic construct
  misc_feature 1..43: note = Human ST6GAL1/ hST6GAL1(Y32) primer
  source 1..43: mol_type = other DNA
aagcttatgtatgattcctttaaattgcaaaccaaggaattcc

SEQ ID NO: 48 - DNA, 33 bp, synthetic construct
  misc_feature 1..33: note = Human ST6GAL1/hST6_Rev primer
  source 1..33: mol_type = other DNA
aaactcgagttagcagtgaatggtccggaagcc

SEQ ID NO: 49 - DNA, 31 bp, synthetic construct
  misc_feature 1..31: note = Human B4GALT1/hB4:ECD_Fwd primer
  source 1..31: mol_type = other DNA
aaaaagcttatgggccgcgacctgagccgcc

SEQ ID NO: 50 - DNA, 31 bp, synthetic construct
  misc_feature 1..31: note = Human B4GALT1/hB4_Rev primer
  source 1..31: mol_type = other DNA
aaactcgagctagctcggtgtcccgatgtcc

SEQ ID NO: 51 - DNA, 40 bp, synthetic construct
  misc_feature 1..40: note = Human ST6GAL1/hST6_SDM_C353A_F primer
  source 1..40: mol_type = other DNA
caagcgcaagactgacgtggcctactactaccagaagttc

SEQ ID NO: 52 - DNA, 40 bp, synthetic construct
  misc_feature 1..40: note = Human ST6GAL1/hST6_SDM_C353A_R primer
  source 1..40: mol_type = other DNA
gaacttctggtagtagtaggccacgtcagtcttgcgcttg

SEQ ID NO: 53 - DNA, 34 bp, synthetic construct
  misc_feature 1..34: note = Human ST6GAL1/hST6_SDM_C364A_F primer
  source 1..34: mol_type = other DNA
gttcttcgatagtgccgccacgatgggtgcctac

SEQ ID NO: 54 - DNA, 34 bp, synthetic construct
  misc_feature 1..34: note = Human ST6GAL1/hST6_SDM_C364A_R primer
  source 1..34: mol_type = other DNA
gtaggcacccatcgtggcggcactatcgaagaac

SEQ ID NO: 55 - DNA, 38 bp, synthetic construct
  misc_feature 1..38: note = IL2 signal sequence/Kozac_Il2ss_Fwd primer
  source 1..38: mol_type = other DNA
aaactcgaggccaccatgtacaggatgcaactcctgtc

(19) United States
(12) Patent Application Publication
(10) Pub. No.: US 2024/0174988 A1
(43) Pub. Date: May 30, 2024

(54) GLYCOENGINEERING

(71) Applicant: The General Hospital Corporation, Boston, MA (US)

(72) Inventors: Robert M. Anthony, Arlington, MA (US); Maya Kitaoka, Arlington, MA (US)

(21) Appl. No.: 18/142,129

(22) Filed: May 2, 2023

Related U.S. Application Data
(63) Continuation of application No. 16/954,814, filed on Jun. 17, 2020, now Pat. No. 11,674,125.
(60) Provisional application No. 62/607,111, filed on Dec. 18, 2017.

(51) Int. Cl. C12N 9/10; A61P 13/12; A61P 19/02; A61P 37/06

(52) U.S. Cl. CPC C12N 9/1081; A61P 13/12; A61P 19/02; A61P 37/06; C12N 9/1051; C12Y 204/01038; C12Y 204/99001; A61K 38/00

(57) ABSTRACT

This disclosure relates to glycoengineering, and methods of utilizing glycoengineering for various therapeutic purposes.

### CLAIM OF PRIORITY

This application is a continuation of U.S. patent application Ser. No. 16/954,814, filed Jun. 17, 2020, which is a § 371 National Stage Application of PCT/US2018/066013, filed Dec. 17, 2018, which claims the benefit of U.S. Provisional Application No. 62/607,111, filed on Dec. 18, 2017. The entire contents of the foregoing are incorporated herein by reference.

### FEDERALLY SPONSORED RESEARCH OR DEVELOPMENT

This invention was made with Government support under Grant No. AR068272 awarded by the National Institutes of Health (NIH). The Government has certain rights in the invention.

### SEQUENCE LISTING

This application contains a Sequence Listing that has been submitted electronically as an XML file named “40978-0040002_SL_ST26.XML.” The XML file, created on Apr. 28, 2023, is 78,216 bytes in size. The material in the XML file is hereby incorporated by reference in its entirety.

### TECHNICAL FIELD

This disclosure relates to glycoengineering, e.g., modulating IgG effector function by engineering antibody glycans in vivo to for various therapeutic effects, e.g., treating IgG-mediated disorders, attenuating autoantibody-mediated inflammation, treating autoimmune diseases, and/or treating antibody-mediated injury during organ transplantation.

### BACKGROUND

The proteins and cells that make up the human body are decorated by sugars (Varki, A. Glycobiology 3, 97-130 (1993)). Sugars can be linked to many types of biological molecule to form glycoconjugates. The enzymatic process that links sugars/saccharides to themselves and to other molecules is known as glycosylation. Glycoproteins, proteoglycans, and glycolipids are the most abundant glycoconjugates found in mammalian cells.

It has been determined that aberrant glycosylation is associated with many different diseases. Thus, there is a need to develop tools and methods to engineer glycosylation, and further use such tools or methods to treat various disorders associated with abnormal glycosylation.

### SUMMARY

This disclosure relates to glycoengineering.

In one aspect, the disclosure relates to a fusion polypeptide having an antibody heavy chain CH2 region; an antibody heavy chain CH3 region; and a catalytic domain of a sialyltransferase, wherein the catalytic domain of the sialyltransferase catalyzes sialylation of a glycoprotein.

In some embodiments, the sialyltransferase is beta-galactoside alpha-2,6 sialyltransferase 1. In some embodiments, the sialyltransferase is a human sialyltransferase.

In some embodiments, the antibody heavy chain CH2 region is a human IgG heavy chain CH2 region. In some embodiments, the antibody heavy chain CH3 region is a human IgG heavy chain CH3 region.

In another aspect, the disclosure relates to a fusion polypeptide having an antibody heavy chain CH2 region; an antibody heavy chain CH3 region; and a catalytic domain of galactosyltransferase, wherein the catalytic domain of galactosyltransferase catalyzes galactosylation of a glycoprotein.

In some embodiments, the galactosyltransferase is beta-1,4-galactosyltransferase 1. In some embodiments, the galactosyltransferase is a human galactosyltransferase.

In some embodiments, the antibody heavy chain CH2 region is a human IgG heavy chain CH2 region. In some embodiments, the antibody heavy chain CH3 region is a human IgG heavy chain CH3 region.

In one aspect, the disclosure provides a polynucleotide encoding the fusion polypeptide as described herein.

In another aspect, the disclosure also provides a vector that has a polynucleotide sequence encoding the fusion polypeptide as described herein.

In one aspect, the disclosure relates to a cell having the vector as described herein, and the vector optionally expresses the fusion polypeptide as described herein.

In one aspect, the disclosure relates to a heteromultimer that has a first fusion polypeptide having an antibody heavy chain CH2 region, an antibody heavy chain CH3 region, and a catalytic domain of sialyltransferase, wherein the catalytic domain of sialyltransferase catalyzes sialylation of a glycoprotein; and a second fusion polypeptide having an antibody heavy chain CH2 region, an antibody heavy chain CH3 region, and a catalytic domain of galactosyltransferase, wherein the catalytic domain of galactosyltransferase catalyzes galactosylation of a glycoprotein.

In some embodiments, the heteromultimer is a heterodimer, and the first fusion polypeptide associates with the second fusion polypeptide, thereby forming the heterodimer.

In some embodiments, the sialyltransferase is beta-galactoside alpha-2,6 sialyltransferase 1. In some embodiments, the sialyltransferase is a human sialyltransferase.

In some embodiments, the galactosyltransferase is beta-1,4-galactosyltransferase 1. In some embodiments, the galactosyltransferase is a human galactosyltransferase.

In one aspect, the disclosure relates to methods of treating a subject having an IgG-mediated disorder. The methods involve administering to the subject an effective amount of a composition having the heteromultimer as described herein.

In some embodiments, the IgG-mediated disorder is inflammation. In some embodiments, the IgG-mediated disorder is an autoimmune disease.

In some embodiments, the autoimmune disease is arthritis. In some embodiments, the autoimmune disease is Goodpasture's disease. In some embodiments, the autoimmune disease is nephrotoxic nephritis. In some embodiments, the autoimmune disease is celiac disease, diabetes mellitus type 1, Graves disease, inflammatory bowel disease, multiple sclerosis, psoriasis, rheumatoid arthritis, or systemic lupus erythematosus.

In another aspect, the disclosure also relates to methods of treating a subject having an IgG-mediated disorder. The methods involve administering to the subject an effective amount of a first polypeptide having a catalytic domain of sialyltransferase and an effective amount of a second polypeptide having a catalytic domain of galactosyltransferase, wherein the catalytic domain of sialyltransferase catalyzes sialylation of a glycoprotein and the catalytic domain of galactosyltransferase catalyzes galactosylation of a glycoprotein.

In some embodiments, the first polypeptide further has an antibody heavy chain CH2 region, and an antibody heavy chain CH3 region.

In some embodiments, the second polypeptide further has an antibody heavy chain CH2 region, and an antibody heavy chain CH3 region.

In some embodiments, the IgG-mediated disorder is inflammation. In some embodiments, the IgG-mediated disorder is an autoimmune disease.

In some embodiments, the autoimmune disease is arthritis. In some embodiments, the autoimmune disease is Goodpasture's disease. In some embodiments, the autoimmune disease is nephrotoxic nephritis. In some embodiments, the autoimmune disease is celiac disease, diabetes mellitus type 1, Graves disease, inflammatory bowel disease, multiple sclerosis, psoriasis, rheumatoid arthritis, or systemic lupus erythematosus.

In one aspect, the disclosure also relates to methods of treating antibody-mediated injury during organ transplantation in a subject. The methods involve administering to the subject an effective amount of a composition having the heteromultimer as described herein.

In another aspect, the disclosure also relates to methods of treating antibody-mediated injury during organ transplantation in a subject. The methods involve administering to the subject an effective amount of a first polypeptide having a catalytic domain of sialyltransferase and an effective amount of a second polypeptide having a catalytic domain of galactosyltransferase, wherein the catalytic domain of sialyltransferase catalyzes sialylation of a glycoprotein and the catalytic domain of galactosyltransferase catalyzes galactosylation of a glycoprotein.

In one aspect, the disclosure also provides a heteromultimer that has a first fusion polypeptide having a collagen trimerizing domain and a catalytic domain of sialyltransferase; a second fusion polypeptide having a collagen trimerizing domain and a catalytic domain of galactosyltransferase; and a third fusion polypeptide having a collagen trimerizing domain, wherein the first fusion polypeptide, the second fusion polypeptide, and the third fusion polypeptide bind to each other, forming the heteromultimer.

In some embodiments, the third fusion polypeptide further has a catalytic domain of sialyltransferase. In some embodiments, the third fusion polypeptide further has a catalytic domain of galactosyltransferase.

In another aspect, the disclosure also relates to a heteromultimer that has a tetramer having four streptavidin polypeptides; and four polypeptides, wherein each of the four polypeptides is linked with biotin, and one or more of the four polypeptides have a catalytic domain of sialyltransferase or a catalytic domain of galactosyltransferase, wherein each of the four polypeptides binds to the tetramer having the four streptavidin polypeptides.

In some embodiments, each of the four polypeptides has a catalytic domain of sialyltransferase or a catalytic domain of galactosyltransferase.

In some embodiments, each of the four polypeptides has a catalytic domain of sialyltransferase. In some embodiments, each of the four polypeptides has a catalytic domain of galactosyltransferase.

In some embodiments, two of the four polypeptides each has a catalytic domain of sialyltransferase, and two of the four polypeptides each has a catalytic domain of galactosyltransferase.

In one aspect, the disclosure also provides a heteromultimer that has an antibody or antibody fragment thereof; a catalytic domain of sialyltransferase; and a catalytic domain of galactosyltransferase, wherein the catalytic domain of sialyltransferase and the catalytic domain of galactosyltransferase each is linked to the antibody or antibody fragment thereof.

In some embodiments, the heteromultimer has an antibody, and the antibody has two antibody heavy chains, and two antibody light chains. In some embodiments, the catalytic domain of sialyltransferase is linked to C-terminus of the antibody heavy chain. In some embodiments, the catalytic domain of sialyltransferase is linked to C-terminus of the antibody light chain. In some embodiments, the catalytic domain of galactosyltransferase is linked to C-terminus of the antibody heavy chain. In some embodiments, the catalytic domain of galactosyltransferase is linked to C-terminus of the antibody light chain.

As used herein, the term “multimer” refers to a protein having two or more polypeptides or a polypeptide complex formed by two or more polypeptides. The polypeptides can associate with each other, forming a quaternary structure.

As used herein, the term “heteromultimer” refers to a multimer having more than one type of polypeptides.

As used herein, the term “homodimer” refers to a multimer having two identical polypeptides.

As used herein, the term “heterodimer” refers to a multimer having two polypeptides, and the two polypeptides are different.

As used herein, the term “luminal domain” or “enzymatic luminal domain” refers to the portion of a glycosylation enzyme that is located within the lumen of the Golgi apparatus in its native state. The enzymatic luminal domain of a glycosyltransferase is usually the soluble portion of the glycosylation enzyme.

As used herein, the term “soluble portion” or “soluble domain” refers to the portion of glycosylation enzyme that is soluble. For trans-Golgi glycosylation enzymes, the soluble portions are often the enzymatic luminal domains of the glycosylation enzymes. For non-trans-Golgi glycosylation enzymes, the entire glycosylation enzymes can be soluble. Thus, in some embodiments, the soluble portion can be the entire glycosylation enzyme or part of the glycosylation enzyme.

As used herein, the term “catalytic domain” refers to a portion of a protein that has a catalytic activity.

As used herein, the term “IgG-mediated disorder” refers to a disorder caused by or characterized by an increased level or an increased activity of Immunoglobulin G (IgG).

As used herein, the term “linked” refers to being covalently or non-covalently associated, e.g., by a chemical bond (e.g., a peptide bond, or a carbon-carbon bond), by hydrophobic interaction, by Van der Waals interaction, and/or by electrostatic interaction.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1E. Solubilizing and engineering glycosyltransferase enzymes. (A-C) Schematics of IgG, its complex, biantennary Fc glycan, and enzyme-Fc fusions are shown. (A) IgG Fab and Fc with a single, N-linked glycosylation site at N297. (B) The glycan core structure (within the box) consists of GlcNAc (squares), mannose (green circles), and variable addition of fucose (red triangle), bisecting GlcNAc, galactose (yellow circles), or sialic acid (purple diamonds). (C) The trans-Golgi enzymes B4GALT1 and ST6GAL1 have cytoplasmic (cyto), transmembrane (TMD), and enzymatic luminal domains (Lumen). ST6GAL1 Cleavage site EFQ41-43 resulting in its secretion is in red. Enzymatic luminal domains of B4GALT1 and ST6GAL1 were fused to IgG Fc (B4^{Fe} and ST6^{Fe}, respectively). (D, E) Linkage-specific lectin blots assaying for glycosyltransferase activity of B4^{Fe}, ST6^{Fc} individually or together. Terminal β1,4 galactose (ECL) or α2,6 sialic acid (SNA) on target glycoproteins fetuin (D), or mouse and human IgG Fcs (E) following incubation with engineered enzymes and sugar-nucleotide donors (UDP-Gal for B4^{Fe}, CMP-SA for ST6^{Fc}).

FIGS. 2A-2H. Anti-inflammatory activity of in vivo sialylation. (A) Mice were treated with K/B×N sera and PBS (black circles), ST6^{Fe} (pink triangles), B4^{Fc} (orange diamonds), or IVIG (blue squares), and paw swelling monitored over several days. (B) Day 10 clinical scores of individual mice in (A) are plotted. (C) K/B×N treated mice were given PBS (black circles), IVIG (blue squares), or B4ST6^{Fe} (red triangles) and paw swelling monitored over several days. (D) Day 9 clinical scores of individual mice from (C) are shown. (E) H&E of paw sections 7 days after induction of arthritis in mice treated with PBS, IVIG, or B4ST6^{Fc}. Blood urea nitrogen (BUN) levels on day 7 (F) and survival (G) of mice induced with NTN, and treated with PBS (black circles), IVIG (blue squares), or B4ST6^{Fe} (red triangles). (H) PAS of stained frozen kidney sections from NTN-treated mice 7 days following PBS, IVIG, or B4ST6^{Fe} administration. Means and standard deviation are plotted. Results are representative of at least two independent repeats. *p<0.05, **p<0.01, ***p<0.005, ****p<0.001, ns (not significant), determined by two-way ANOVA followed by Tukey's posthoc test.

FIGS. 3A-3G. Receptor requirements for in vivo sialylation. (A) FcγRIIB^{-/-} mice were given K/B×N sera and PBS (black circles), IVIG (blue squares), or B4ST6^{Fe} (red triangles) and paw swelling monitored over several days. (B) Day 6 clinical scores of individual mice from (A) are plotted. (C) WT mice were administered TKO SIGN-R1 antibody and K/B×N sera and PBS (black circles), IVIG (blue squares), or B4ST6^{Fe} (red triangles) and paw swelling monitored over several days. (D) Day 6 clinical scores of individual mice from (C) are shown. (E) SIGN-R1^{-/-} and (F) hDC-SIGN⁺/SIGN-R1^{-/-} mice were given K/B×N sera and PBS (circles), IVIG (squares), or B4ST6^{Fe} (triangles) and paw swelling monitored over several days. (G) Day 6 clinical scores of SIGN-R1^{-/-} and hDC-SIGN⁺/SIGN-R1^{-/-} mice are shown. Means and standard deviation are plotted. Results are representative of at least two independent repeats. **p<0.01, ***p<0.005, ****p<0.001, ns (not significant), determined by two-way ANOVA followed by Tukey's posthoc test.

FIGS. 4A-4E. Enzymatic requirements for in vivo sialylation. (A) Schematics of B4GALT1 and enzymatically dead ST6GAL1 (C350A, C361A) and the resulting B4ST6^{Fc}_{CACA}. (B) Schematic of removal of the Fc glycan on B4^{Fe} and ST6^{Fe} following EndoS-treatment resulting in B4ST6Fc-Endo. (C) Linkage-specific lectin blots assaying for terminal β1,4 galactose (ECL) or α2,6 sialic acid (SNA) on human IgG Fcs following incubation with B4ST6^{Fe}, B4ST6^{Fc}_{CACA}, or B4ST6^{Fc}-Endo. Galactosylation was assayed by incubation with UDP-Gal on (G0) IgG Fc. Sialyltransferase activity was evaluated by incubation with CMP-SA on (G2) IgG Fc. (D) WT mice were administered K/B×N sera and PBS (black circles), IVIG (blue squares), or B4ST6^{Fe} (red triangles), B4ST6^{Fe}_{CACA} (black crosses, red dotted line), or B4ST6Fc-Endo (red triangle with black edge, red dotted line) and paw swelling monitored over several days. (E) Day 6 clinical scores of mice from (D) are shown. Means and standard deviation are plotted. Results are representative of at least two independent repeats. *p<0.05, **p<0.005, ****p<0.001, ns (not significant), determined by two-way ANOVA followed by Tukey's posthoc test.

FIGS. 5A-5E. Characterizing in vivo sialylation during autoimmune inflammation. (A, B) HPLC traces of total glycans recovered from serum or kidney IgG in day 7 NTN-treated mice following administration of PBS, IVIG, or B4ST6^{Fc} Shading corresponds to retention time of terminal sugar (blue, G0; yellow, G1; orange, two G2; pink, one S1; purple, S2). (C, D) Ratios of mono-sialylated and agalactosylated glycans (S1/G0) from IgG described in (A, B). (E) Total IgG purified from serum and kidney of NTN-treated mice received PBS, IVIG, or B4ST6^{Fe} was probed by immunoblotting for mouse and human IgG. Means and standard deviation are plotted. Results are representative of at least two independent repeats. **p<0.01, ns (not significant), as determined by two-way ANOVA followed by Tukey's posthoc test.

FIGS. 6A-6H. Platelet activation and in vivo sialylation. (A) Kidneys of untreated and day 7 NTN-treated mice following treatment with PBS, IVIG and B4ST6^{Fe} were examined for glomeruli (mNephrin, green), mouse IgG (blue), platelets (CD41, red), activated platelets (CD62, yellow). Representative individual and overlaid images are shown. (B, C) Untreated and clopidogrel-treated mice were given K/B×N sera and PBS (black circles), IVIG (blue squares), or B4ST6^{Fe} (red triangles) and paw swelling monitored over several days. (D) Day 6 clinical scores of untreated and clopidogrel-treated mice are shown. NTN was induced in untreated (E, F) and clopidogrel-treated (G, H) animals, and day 7 BUN levels (mg/dL) and survival was monitored. Means and standard deviation are plotted. Results are representative of at least two independent repeats. *p<0.05, ***p<0.005, ****p<0.001, ns (not significant), determined by two-way ANOVA followed by Tukey's posthoc test.

FIGS. 7A-7D. Therapeutic in vivo sialylation. (A, B) Human platelets plasma were untreated, activated (Thrombin+), or activated after clopidogrel treatment (Thrombin+, Clopidogrel+), and assayed for UPD-Gal (A) and CMP-SA (B). (C-D) Mice were treated with K/B×N sera on day 0 and PBS (black circles), IVIG (blue squares), or B4ST6^{Fe} (red triangles), or day 3 and paw swelling monitored (C) over several days. (D) Day 7 clinical scores of individual mice from (C) are shown. Means and standard deviation are plotted. Results are representative of at least two independent repeats. **p<0.01, ***p<0.005, ****p<0.001, ns (not significant), determined by two ANOVA followed by Tukey's posthoc test.

FIGS. 8A-8D. Engineering and characterization of soluble galactosyl- and sialyltransferase proteins. (A) Protein sequence of human ST6GAL1. Yellow and light blue shaded sequences represent cytosolic and transmembrane domain (TMD), respectively. Numbered red triangles indicate the start site of the sialyltransferase which was fused to IgG Fc. The triangle with a star indicates the start site of the soluble ST6GAL1 that was used in all experiments in this manuscript. (B) Schematic representations of each ST6GAL1 are shown. (C) Immunoblots of Fc-enzyme proteins for reactivity to IgG (N, native protein; D, denatured protein). ST6^{Fc}s were cleaved to Fc and ST6GAL1 upon denaturation when fused upstream of EFQ41-43. (D) Immunoblots of ST6^{Fe}, B4^{Fe}, and B4ST6^{Fe} for reactivity to B4GALT1, ST6GAL1, or IgG.

FIGS. 9A-9B. Anti-sheep response after NTN-induction. (A, B) NTN-induced mice were treated with PBS (black circles), high dose IVIG (blue squares), or B4ST6^{Fe} (2.5 mg/kg) (red triangles). Day 7 anti-sheep IgG titers were determined by ELISA.

FIGS. 10A-10B. Receptor requirements of in vivo sialylation control groups. (A, B) Day 6 clinical scores of untreated C57BL/6 mice after K/B×N injection for Figure. 2B and 2D, respectively. These are from control groups for FcγRIIB^{-/-} (A) and TKO-SIGN-R1 (B) treatments shown in FIGS. 3A-3D. Results are representative of at least two independent repeats. *p<0.05, **p<0.01, ***p<0.005, ****p<0.001, ns (not significant), determined by two-way ANOVA followed by Tukey's posthoc.

FIGS. 11A-11D. In vivo sialylation during homeostasis. Serum concentrations of IVIG (A) and B4ST6^{Fc} (B) at defined intervals after administration are plotted with half-lives inset. (C) Blood test values following administration of PBS (black circles), B4ST6^{Fe} one week earlier (red triangles), or B4ST6^{Fe} two months earlier (open red triangles). WBC, white blood cell; LYM, lymphocytes; MONO, monocytes; GRAN, granulocytes; HCT, hematocrit; MCV, mean corpuscular volume; RDW, red blood cell distribution width; HGB, hemoglobin; MCHC, mean corpuscular hemoglobin concentration; MCH, mean corpuscular hemoglobin; RBC, red blood cell (erythrocyte) count; PLT, platelet; MPV, mean platelet volume; BUN, blood urea nitrogen; ALT (GPT), Alanine Amino Transferase; ALP, alkaline phosphatase; GGT, Gamma-Glutamyl Transferase (D) Ratios of mono-sialylated and agalactosylated glycans (S1/G0) are plotted following administration of PBS, B4ST6^{Fe} one week earlier, or B4ST6^{Fe} two months earlier.

FIG. 12. Site-specific in vivo sialylation during inflammation. Ratios of disialylated and agalactosylated glycans (S2/G0) IgG recovered from kidney of NTN-induced mice.

FIGS. 13A-13B. Platelets during in vivo sialylation of NTN. (A) Kidneys of untreated and 7 days after NTN induction in PBS, IVIG and B4ST6^{Fc} treated animals were examined for glomeruli (mNephrin, green), DAPI (blue), platelets (CD41), activated platelets (CD62). Representative individual and overlayed images are shown. (B) Day 7 anti-sheep IgG titers of NTN-induced mice were treated with PBS (black circles), high dose IVIG (blue squares), or B4ST6^{Fe} (2.5 mg/kg) (red triangles) some of which also received clopidogrel.

FIGS. 14A-14B. Preventative and therapeutic in vivo sialylation. (A) K/B×N treated mice were given PBS (black circles), IVIG (blue squares), or B4ST6^{Fc} (red triangles) on day 0 and paw swelling monitored over several days. (B) Day 7 clinical scores of individual mice are shown. These are control groups for data shown in FIGS. 7C and 7D.

FIG. 15A is a diagram showing a soluble portion of a glycosylation enzyme.

FIG. 15B is a diagram showing a dimer formed by soluble portions of glycosylation enzymes that are fused to IgG Fcs.

FIG. 15C is a diagram showing a trimer formed by soluble portions of glycosylation enzymes that are fused to collagen trimerizing domains.

FIG. 15D is a diagram showing a tetramer formed by soluble portions of glycosylation enzymes that are biotinylated by biotin ligase and subsequently incubated with streptavidin (SA).

FIG. 16A is a diagram showing soluble portions of glycosylation enzymes that are fused to the C-terminus of the heavy chains of an antibody.

FIG. 16B is a diagram showing soluble portions of glycosylation enzymes that are fused to the C-terminus of the light chains of an antibody.

FIG. 17 is a diagram showing engineered glycol-enzymes can be used to treat inflammation.

FIG. 18 shows western blot results (with anti-human IgG) and Coomassie Gel stain of the B4ST6^{Fe} with and without “knobs-into-holes” mutations, ST6^{Fc} homodimers, and B4^{Fe} homodimers.

FIG. 19 shows the effects of original B4ST6^{Fe}, heterodimeric B4ST6Fc^{Kln}, and B4ST6Fc^{G3} in arthritis models.

FIG. 20 lists the amino acid sequences of several exemplary glycosylation enzymes.

FIG. 21 lists the amino acid sequences of exemplary fragment crystallizable region (Fc) of several human and mouse immunoglobulin G (IgG).

FIG. 22 lists the amino acid sequences of several exemplary glycosylation enzyme-Fc fusion proteins.

FIG. 23 lists the amino acid sequences of dog IgG heavy chain A, dog IgG heavy chain B, dog IgG heavy chain C, dog IgG heavy chain D, dog ST6GAL1, and dog B4GALT1.

FIG. 24 lists the amino acid sequences of cat IgG1a heavy chain, cat IgG1b heavy chain, cat ST6GAL1, and cat B4GALT1.

FIG. 25 lists the amino acid sequences of cow IgG1 heavy chain constant region, cow IgG2 heavy chain constant region, cow IgG3 heavy chain constant region, cow ST6GAL1, and cow B4GALT1.

FIG. 26 lists the amino acid sequences of horse IgG1 heavy chain constant region, horse IgG2 heavy chain constant region, horse IgG3 heavy chain constant region, horse IgG4 heavy chain constant region, horse IgG5 heavy chain constant region, horse IgG6 heavy chain constant region, horse ST6GAL1, and horse B4GALT1.

### DETAILED DESCRIPTION

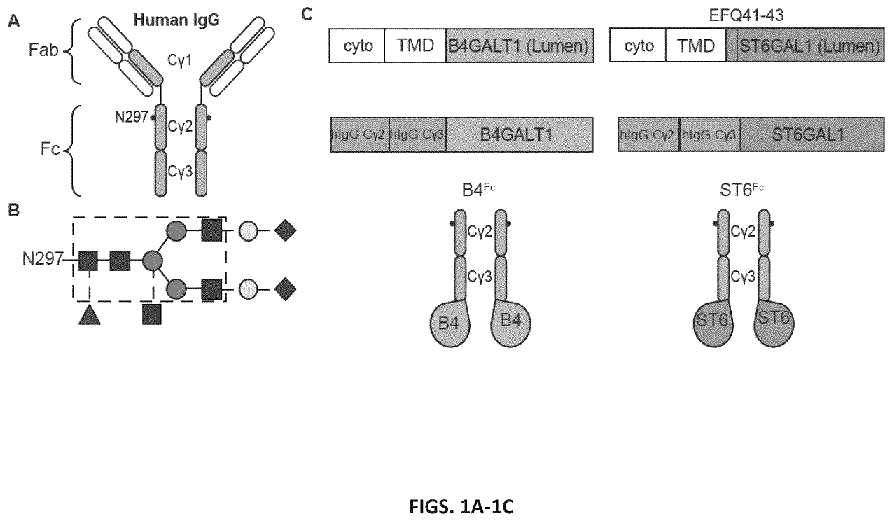

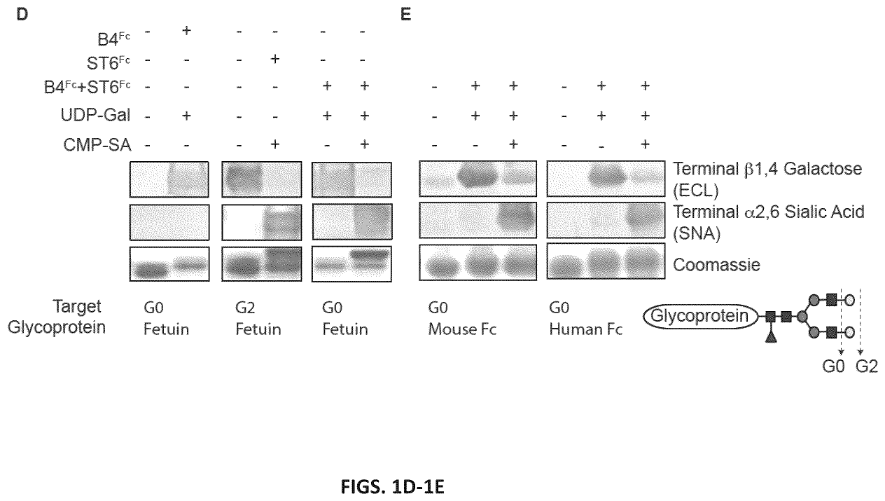

Immunoglobulin gamma (IgG) antibodies are the preeminent effector proteins of the immune system. They are essential following pathogen exposure or vaccination, bridging the adaptive and innate immune system for clearance of microbes, but can also contribute to the pathogenesis of autoimmune diseases when they are generated against self-antigens (Nimmerjahn and Ravetch, 2008b). The bimodal activity of IgG antibodies allows simultaneous recognition of antigen by the antigen-binding fragment (Fab, FIG. 1A) with high affinity, and recruitment and activation of leukocytes through interactions between the crystallizable fragment (Fc, FIG. 1A) and Fc gamma receptors (FcγRs) expressed by innate immune cells, or the initiator of the complement cascade, C1q (Nimmerjahn et al., 2015). This triggers the canonical inflammatory effector functions of IgG, such as antibody-dependent cytotoxicity (ADCC), uptake of recognized antigens, and complement-dependent cytotoxicity (CDC) (Franklin, 1975, Huber et al., 1976).

A single N-linked glycan is present on each heavy chain of all IgG, positioned at asparagine-297 in the Fc (N-297, FIGS. 1A-1B) (Arnold et al., 2007). The core heptasaccharide of the glycan has a complex biantennary structure that can vary by the addition of fucose, N-acetylglucosamine (G1cNAc), galactose, or sialic acid (FIG. 1B). These variable additions account for tremendous heterogeneity, with over 30 distinct glycans identified on circulating IgG in healthy individuals (Kaneko et al., 2006b). Importantly, studies over the last decade have demonstrated the composition of the Fc glycan exerts profound influence over IgG effector functions (Jefferis, 2005, Jefferis, 2009a, Jefferis, 2009b). IgG with afucosylated Fc glycans have 50-fold enhanced affinity to the activating FcγR, FcγRIIIA, compared to fucosylated IgG, and exhibit markedly enhanced ADCC in vivo (Ferrara et al., 2011, Natsume et al., 2005, Okazaki et al., 2004, Shields et al., 2002). As a consequence, some next-generation therapeutic IgG intended to elicit ADCC are being engineered to lack fucose (Beck et al., 2010). Indeed, recent studies have extended these findings to infectious diseases, identifying an association of dengue-specific IgG with afucosylated Fc glycans with dengue hemorrhagic fever (Wang et al., 2017), and afucosylated IgG in controlling latent, and not active, TB infections (Lu et al., 2016). The most successful HIV vaccine trial to date resulted in increased levels of bisecting GlcNAc on IgG Fc glycans, a modification that also increased affinity to FcγRIIIA, albeit to a lesser extent than afucosylation (Ackerman et al., 2013, Chung et al., 2014, Davies et al., 2001). Conversely, terminal sialylation of the Fc glycan reduces IgG affinity for type I FcγRs, and sialylated IgG have reduced capacity to initiate ADCC in vivo (Scallon et al., 2007, Anthony et al., 2008a, Li et al., 2017). Enhanced sialylation on IgG following influenza vaccination was attributed to improved affinity maturation through a type II FcγR-CD23, pathway (Wang et al., 2015). Although the regulation of IgG glycosylation is not completely understood, IL-23 has been implicated in regulating expression of ST6GAL1 (Pfeifle et al., 2017).

Paradoxically, IgG is commonly used in the clinic to suppress inflammation (Negi et al., 2007). Intravenous immunoglobulin (IVIG) is a therapeutic preparation of polyclonal, monomeric IgG derived from tens of thousands of healthy donors and has successfully been used in the clinic for almost 40 years at a high dose (1-2 g/kg) for the treatment of inflammatory and autoimmune diseases (Imbach et al., 1981, Nimmerjahn and Ravetch, 2008a). Mechanistic studies revealed that the Fc portion of IVIG was sufficient for anti-inflammatory activity in vivo (Debre et al., 1993, Samuelsson et al., 2001), and that this required inhibitory FcγRIIB (Samuelsson et al., 2001, Schwab et al., 2012, Schwab et al., 2014, Tackenberg et al., 2009, Tackenberg et al., 2010). Further studies demonstrated sialylation of the Fc glycan was essential for this activity (Kaneko et al., 2006b, Anthony et al., 2008a). Instead of binding the activating type I FcγRs, sialylated IgG Fc bound type II FcγRs, human DC-SIGN or murine SIGN-R1, culminating in increased surface expression of the inhibitory FcγRIIB on inflammatory effector cells (Anthony et al., 2011, Anthony et al., 2008b, Samuelsson et al., 2001). Thus, the IgG Fc glycan composition, and specifically terminal sialic acid, along with DC-SIGN and FcγRIIB are responsible for the anti-inflammatory activity of IgG in vivo (Kaneko et al., 2006b, Tackenberg et al., 2009, Anthony et al., 2011, Washburn et al., 2015).

This disclosure relates to methods and compositions comprising a fusion peptide comprising a catalytic domain of a glycosylation enzyme fused to Fc (e.g., glycosylation enzyme-Fc fusion proteins). The methods and compositions described herein can be used to modulate IgG effector function by engineering antibody glycans in vivo for various therapeutic effects. For example, this disclosure relates to modulation of IgG effector function by engineering antibody glycans in vivo as a novel means to attenuate autoantibody-mediated inflammation. It is well established that glycosylation, including sialylation, profoundly affects IgG biology. Indeed, the contribution of IgG glycosylation to infectious diseases is increasingly appreciated, and IgG glycoforms are reported to contribute to the clinical presentation of Dengue fever and Tuberculosis (Lu et al., 2016, Wang et al., 2017). Afucosylated IgG with enhanced affinity for activating FcγRIIIA were found in patients more likely to suffer from dengue hemorrhagic fever, and also in patients with latent but not active TB infections. Sialylation markedly reduces the affinity of IgG to FcγRs, rendering IgG unable to trIgGer inflammation (Scallon et al., 2007, Kaneko et al., 2006b, Anthony et al., 2008a, Li et al., 2017). Enhanced sialylation on influenza-specific IgG Fc glycans was found following vaccination, and was associated with flu-specific broadly neutralizing antibodies in a CD23-type II FcγR dependent manner (Wang et al., 2015). Also, sialylated IgG Fcs convey anti-inflammatory activity when administered at a sufficiently high dose (Anthony et al., 2008a, Kaneko et al., 2006b, Washburn et al., 2015).

The mechanisms governing the dose-dependent anti-inflammatory actions of IgG have been extensively debated (Clynes, 2007, Schwab and Nimmerjahn, 2013). However, functional tests of IVIG have consistently shown that sialylation of IgG is responsible for this anti-inflammatory activity in vivo (Washburn et al., 2015, Zhang et al., 2016, Fiebiger et al., 2015, Schwab et al., 2012, Schwab et al., 2014, Ohmi et al., 2016). Removal of the Fc glycan from IgG rendered IVIG unable to suppress autoimmune inflammation (Kaneko et al., 2006b). Moreover, IVIG treated with neuraminidase, to removed terminal sialic acid from the Fc glycan, also exhibited no anti-inflammatory activity (Kaneko et al., 2006b). Thus, the IgG Fc glycan composition, and specifically sialic acid, is responsible for the anti-inflammatory activity of IgG in vivo.

Generation of sialylated IgG Fcs is not trivial, and has likely contributed to confusion in the literature. Indeed, contaminating LPS, degradation of Fcs, improper lectin-enrichment (Stadlmann et al., 2009), irrelevant in vitro assays (Bayry et al., 2009), and heterogeneity of Fab-specificity in IVIG have confounded results. Further, characterization of the sialylated material provides an explanation for some of the disparity, as ST6GAL1 can both attach and remove sialic acid (Washburn et al., 2015). Importantly, a number of groups have reported findings similar to original reports that sialylated hIgG1 is in fact anti-inflammatory in experimental models of Guillain-Barre syndrome (Zhang et al., 2016), and collagen-induced arthritis (Ohmi et al., 2016). Of note, through sialylating IgG in vivo, many of the technical issues of generating anti-inflammatory sialylated IgG have been circumvented.

Successful glycoengineering in vivo efforts have used bacterial-derived glycan-modifying enzymes (Albert et al., 2008, Xiao et al., 2016). The Streptococcal endoglycosidase, EndoS, has been demonstrated to attenuate IgG-mediated inflammation (Albert et al., 2008). Also, a Vibrio cholerae neurmaminidase was targeted to tumor glycocalixes to improve ADCC (Xiao et al., 2016). These studies demonstrated the power of glycoengineering, however repeated administration of these drugs may prove difficult because of immune responses targeting the bacterial-derived enzymes. While it is possible that B4ST6^{Fe} will be targeted by the immune response, other human IgG Fc fusions have been well tolerated.

The potential applications for in vivo sialylation extend well beyond autoimmune and inflammatory conditions. Indeed, these methods can be applied to conditions currently treated by high dose IVIG. But modulation of IgG sialylation could also be utilized to improve vaccine efficacy, as studies have reported that initial IgG generated following vaccination are sialylated, and these sialylated IgG contribute to improved affinity maturation through a type II FcγR-dependent mechanism (Wang et al., 2015). Furthermore, in vivo sialylation can be effective at truncating the activity of therapeutic IgG at defined intervals after treatment. Also, these glycosyltransferases and the Fc portion of these fusion proteins can be further engineered, including increased FcRn affinity to extended serum half-life or increased/decreased receptor binding (Schlothauer et al., 2016).

Therefore, this disclosure relates the functions of sialylation on IgG biology by fusing human glycosylation enzymes found in the trans-Golgi to IgG Fcs. Efficient sialylation required both enzymes that attach galactose and sialic acid (Anthony et al., 2008a). Importantly, this combination of glycosylation enzymes is able to attenuate autoantibody-mediated inflammation in two distinct models in vivo, by selectively sialylating IgG deposited at the site of inflammation. Administration of this enzyme combination does not appear to affect sialylation of IgG in circulation, or that of other glycoproteins in circulation. The data show that this is likely because platelets release galactose and sialic acid substrates only at sites of inflammation. Thus, the present disclosure provides a potent approach to attenuate harmful autoantibody-mediated inflammation through glycoengineering endogenous antibodies and converting them to anti-inflammatory mediators.

The methods and compositions (e.g., glycosylation enzyme-Fc fusion proteins) described herein can also be used for various other therapeutic effects, e.g., treating IgG-mediated disorders, treating autoimmune diseases, or treating antibody-mediated injury during organ transplantation. Further, the glycosylation enzyme-Fc fusion proteins as described in the present disclosure are well tolerated, as Complete Blood Count (CBC) and Comprehensive Metabolic Panel (CMP) analysis are within normal levels both one week and two months after administration, making them suitable for clinic use. Thus, this disclosure provides a useful approach to treat IgG-mediated disorders (e.g., inflammation, and autoimmune diseases) and antibody-mediated injury during organ transplantation.

### Glycans

Glycans have an important role in the function of many proteins. Glycans are saccharides (i.e., a plurality of monosaccharides linked glycosidically) that form the carbohydrate portion of glycoconjugates (e.g., glycoproteins, glycopeptides, peptidoglycans, glycolipids, glycosides and lipopolysaccharides). They can be added to proteins in the endoplasmic reticulum, and further modified as proteins travel through the Golgi apparatus. Precursor glycan structures can be attached to asparagine (N-linked), serine or threonine (O-linked), phospholipids (GPI), tryptophan (C linked), or by phosphodiester bonds (phosphoglycosylation). While many biological functions are ascribed to glycans, two general types of functions of glycosylation are (1) maintaining or modifying the structural or functions of glycoproteins, and (2) providing a glycan-based recognition platform for carbohydrate receptors or lectins.

### N-Linked Glycosylation

In the lumen of the endoplasmic reticulum, a 14-residue precursor oligosaccharide (glucose₃ mannose₉N-acetylglucosamine₂, (Glc₃Man₉GlcNAc₂)) is transferred to an asparagine residue found in the consensus sequence N-X-S/T by the enzyme oligosaccharyltransferase (Schachter et al., in Essentials of Glycobiology, Edn. 2010/03/20, eds. V. A et al., Cold Spring Harbor Laboratory Press, Cold Spring Harbor (NY), 2009). The precursor glycan is trimmed to a high-mannose structure (Man₈₋₉GlcNAc₂) by exoglycosidases while the protein being synthesized is assembled and transported to the Golgi. The glycan can then be further processed as a protein progresses through the secretory pathway.

The core complex biantennary glycan structure (GlcNAc₃Man₉GlcNAc₂) is generated by the transfer of GlcNAc by β1,2 N-acetylglucosaminyltransferase-II. This structure is found in the constant region of IgG antibodies, and can be further modified. The core GlcNAc is available for fucosylation by α1,6-fucosyltransferase. Bisecting N-acetylglucosamine is attached to the core by N-acetylglucosaminyltransferase-III. As a protein progresses along the secretory pathway, the glycan can be further modified in the trans-Golgi by the addition of galactose and sialic acid to the arms by β1,4 galactosyltransferase and α2,6 sialyltransferase, respectively.

### O-Linked Glycosylation

O-linked glycosylation is initiated by the attachment of GlcNAc to serine or threonine (Schachter et al., in Essentials of Glycobiology, Edn. 2010/03/20, eds. V. A et al., Cold Spring Harbor Laboratory Press, Cold Spring Harbor (NY), 2009). The pathway responsible for these modifications is less understood than N-linked glycosylation. An estimate suggests at least eight mammalian N-acetylglucosaminyltransferase enzymes exist, which are differentially expressed depending on cell type. Thus, it is not clear whether the ER or Golgi is the initial site for this glycosylation. No known consensus amino acid sequence exists for O-linked glycans, although secondary structural preferences are suggested instead. In fact, most O-glycosylation is found in β turns. Further, enrichment for proline residues is noted at the −1 and +3 positions, while charged residues are not favored at these positions.

Most O-glycans contain a Core 1 subtype structure formed by the addition of galactose in a β1-3 linkage to the GlcNAc by the Core 1 β1-3 galactosyltransferase (Core 1 GalT). Core 2-type O-glycans can be generated by addition of GlcNAc to the GalNAc in a β1-6 linkage. Core 2 O-glycans require the Core 1 structure as a substrate. Generation of the Core 3 O-glycan is controlled by Core 3 GlcNAcT activity, which uses the GlcNAc-serine/threonine substrate. It is possible that competition between Core 1 GalT and Core 3 GalT enzymes in some cell types or among certain glycoproteins regulates Core 1 and Core 3 generation.

### GPI-Linked Glycosylation

The de novo biosynthesis of glycosphingolipids begins on the inner leaflet of membranes in the ER-Golgi pathway (Schachter et al., in Essentials of Glycobiology, Edn. 2010/03/20, eds. V. A et al., Cold Spring Harbor Laboratory Press, Cold Spring Harbor (NY), 2009). Ceramide is first synthesized, and then glucosylated or galactosylated by specific glycosyltransferases. The addition of a β-linked galactose residue follows. Thereafter, the molecules can be elongated in a stepwise fashion, giving a wide variety of different core structures. The outer extensions of glycosphingolipids, including the addition of sialic acids, fucose, or glucuronic acid residues share much in common with those of N- and O-glycans.

### Glycosylation Enzymes

Glycosylation enzymes are responsible for the reaction in which a carbohydrate, i.e. a glycosyl donor, is attached to a hydroxyl or other functional group of another molecule (a glycosyl acceptor, e.g., proteins, lipids, and glycans). The soluble portions (or the enzymatic luminal domains) or the catalytic domains of these glycosylation enzymes can be fused with Fc, or other appropriate peptides to form multimers, and can be used in any methods described herein.

### α-2,6 Sialyltransferase (ST6GAL1)

Attachment of sialic acid to galactose on IgG Fc glycans is catalyzed by the enzyme α-2,6 sialyltransferase (ST6GAL1; NP_003023.1; SEQ ID NO: 1). Sialylation of IgG by ST6GAL1 typically occurs in the trans-Golgi where ST6GAL1 is found anchored in the Golgi by a transmembrane domain (TMD, FIG. 1C). This trans-Golgi enzyme can attach terminal sialic acid to complex biantennary glycans. As shown in FIG. 1C, the trans-Golgi enzyme ST6GAL1 has a cytoplasmic domain (cyto), a transmembrane domain (TMD), and an enzymatic luminal domain (Lumen) (amino acids 24-406 of SEQ ID NO:1, amino acids 29-406 of SEQ ID NO:1, amino acids 34-406 of SEQ ID NO: 1, or amino acids 41-406 of SEQ ID NO: 1). The catalytic domain (amino acids: 160-390 of SEQ ID NO: 1) is located within the enzymatic luminal domain.

Several distinct promoters regulate the cellular and tissue specific expression of this transferase (Kalcheva et al. Mammalian genome: official journal of the International Mammalian Genome Society 8, 619-620 (1997); Wang et al. The Journal of biological chemistry 268, 4355-4361 (1993)). For example, promoter 1 is used exclusively to express ST6GAL1 by hepatocytes, while B cells use promoter 2 (Appenheimer, M. M. et al. Glycobiology 13, 591-600 (2003)). Hepatocytes are responsible exclusively for production of soluble ST6GAL1 (sST6GAL1), which is cleaved and secreted into the circulation. Importantly, genetic disruption of liver-specific promoter 1 resulted markedly reduced levels of circulating sialylated IgG, compared to wild type controls. However, B cells from both mouse strains were able to express the enzyme, implicating the hepatic soluble form of this enzyme in IgG sialylation.

### β-1,4-Galactosyltransferase 1 (B4GALT1)

Beta-1,4-galactosyltransferase 1 is an enzyme that is encoded by the B4GALT1 gene in humans. B4GALT1 (NP_001488.2; SEQ ID NO: 2) is a type II membrane-bound glycoprotein that appear to have exclusive specificity for the donor substrate UDP-galactose. It transfers galactose in a beta1,4 linkage to similar acceptor sugars (e.g., N-Acetylglucosamine (GlcNAc)) that are either monosaccharides or the non-reducing ends of glycoprotein carbohydrate chains. The catalytic domain (amino acids: 185-390 of SEQ ID NO: 2) is located within the luminal domain (amino acids 79-398 of SEQ ID NO: 2, or amino acids 106-398 of SEQ ID NO: 2) of B4GALT1.

### Soluble Glycosylation Enzymes

Many glycosylation enzymes are secreted. The soluble forms can be derived, e.g., from their membrane-associated forms by proteolytic cleavage near the transmembrane (TM) region, in the carboxyl-terminal direction.

As shown in FIG. 1C, the trans-Golgi enzymes B4GALT1 and ST6GAL1 include cytoplasmic domains (cyto), transmembrane domains (TMD), and enzymatic luminal domains (Lumen). The single hydrophobic segment serves as a signal-anchor sequence. This transmembrane segment (TMD) spans the lipid bilayers of the secretory pathway, including the membrane of the Golgi apparatus. The enzymatic luminal domain of a glycosyltransferase is located within the lumen of the Golgi apparatus. Membrane-tethered transferases are susceptible to proteolytic cleavages within its “stem” region. Proteolysis liberates a catalytically active, soluble form of the enzyme that may be released from the cell. As a consequence, many glycosylation enzymes are found in soluble form in the circulation and in various body fluids. Intriguingly, the production of these soluble enzymes in hepatocytes and endothelium can also be dramatically increased under certain inflammatory conditions.

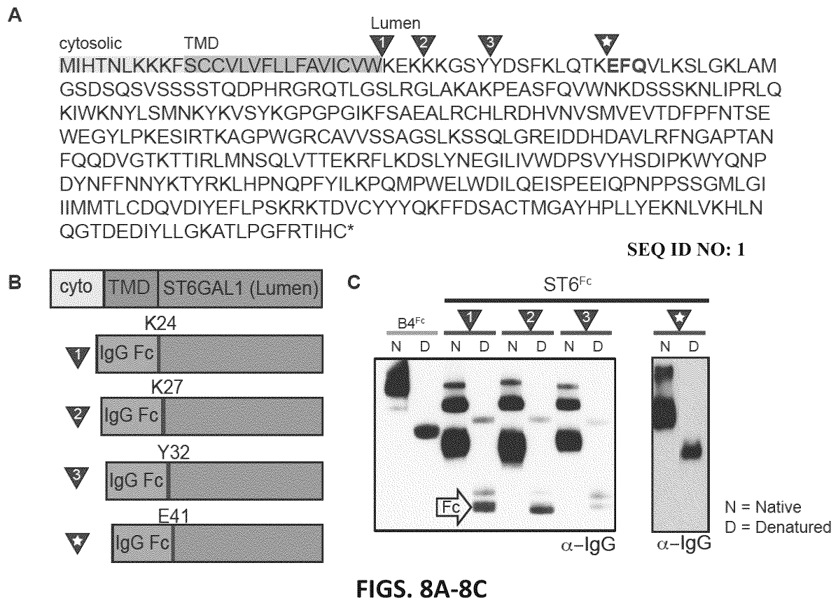

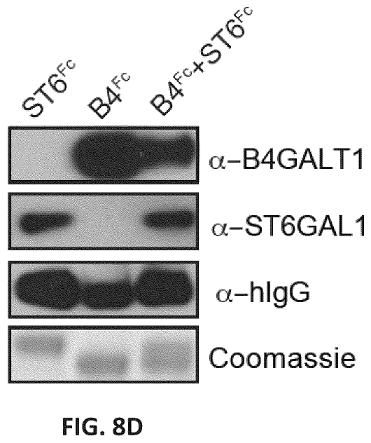

ST6GAL1 has a β-Secretase (BACE1) cleavage site in its luminal domain at EFQ41-43, which can result in its secretion (FIG. 1C, FIGS. 8A-8D). The soluble ST6GAL1 is enzymatically active in the circulation, and can contribute to sialylation of IgG Fc glycans.

The cleavage site for B4GALT1 is L79 and S106. A subpopulation of this enzyme is secreted following proteolytic cleavage in its stem domain, and the soluble form of this enzyme is enzymatically active.

Thus, in one aspect, this disclosure provides a fusion protein or a peptide comprising or consisting of the enzymatic luminal domain of sialyltransferase or the catalytic domain of sialyltransferase. In some embodiments, the enzymatic luminal domain of sialyltransferase does not have BACE1 cleavage site. In some embodiments, the enzymatic luminal domain of sialyltransferase comprises or consists of amino acids 24-406 of SEQ ID NO: 1, amino acids 29-406 of SEQ ID NO: 1, amino acids 34-406 of SEQ ID NO: 1, or amino acids 41-406 of SEQ ID NO: 1. The catalytic domain of sialyltransferase catalyzes sialylation of a glycoprotein. In some embodiments, the catalytic domain of sialyltransferase comprises or consists of amino acids 160-390 of SEQ ID NO: 1.

This disclosure also provides a fusion protein or a peptide comprising or consisting of the enzymatic luminal domain of galactosyltransferase or the catalytic domain of galactosyltransferase. In some embodiments, the enzymatic luminal domain of galactosyltransferase comprises or consists of amino acids 79-398 of SEQ ID NO: 2, or amino acids 106-398 of SEQ ID NO: 2. The catalytic domain of galactosyltransferase catalyzes galactosylation of a glycoprotein. In some embodiments, the catalytic domain of galactosyltransferase comprises or consists of amino acids 185-390 of SEQ ID NO: 2.

### Nucleic Acid Sequences and Amino Acid Sequences

This disclosure provides various nucleic acid sequences and amino acid sequences.

In some embodiments, the nucleic acid sequence is at least 60%, 65%, 70%, 75%, 80%, 85%, 90%, 91%, 92%, 93%, 94%, 95%, 96%, 97%, 98%, 99% identical to any of the nucleic acid sequences disclosed herein. In some embodiments, the nucleic acid sequence is identical to any of the sequences described in this disclosure.

In some embodiments, the amino acid sequence is at least 60%, 65%, 70%, 75%, 80%, 85%, 90%, 91%, 92%, 93%, 94%, 95%, 96%, 97%, 98%, 99% identical to any of the amino acid sequences disclosed herein. In some embodiments, the amino acid sequence is identical to any of the sequences described in this disclosure.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

### FcB4, FcST6, and FcChm

Golgi enzymes, including ST6GAL1 and B4GALT1, are Golgi type II membrane proteins. The enzymatic transferase activity is located in the C-terminus of the protein (FIG. 1C).

The glycosylation enzyme, enzymatic luminal domain, soluble domain, or the catalytic domain thereof can be fused to IgG (e.g., IgG1, IgG2, IgG3, IgG4) or a part thereof. In some embodiments, the glycosylation enzyme, enzymatic luminal domain, soluble domain, or the catalytic domain thereof can be used to the Fc portions of IgG (e.g., IgG1, IgG2, IgG3, IgG4). Fc fusions have a number of advantageous: the soluble protein will have an extended serum half-life (e.g., more than 5 days, 10 days, 14 days, or 20 days), and also will form a dimer. In some embodiments, these fusion polypeptides can form homodimers or heterodimers, depending on the glycosylation target.

The IgG Fc can be the Fc region of any IgG known in the art. For example, the IgG Fc can be a human IgG1-Fc (e.g., comprising amino acids 26-256 of SEQ ID NO: 3), a human IgG2-Fc (e.g., comprising amino acids 52-266 of SEQ ID NO: 4), a human IgG3-Fc (e.g., comprising amino acids 41-318 of SEQ ID NO: 5), a human IgG4-Fc (e.g., comprising amino acids 53-268 of SEQ ID NO: 6), a mouse IgG1-Fc (e.g., comprising amino acids 26-252 of SEQ ID NO: 7), a mouse IgG2a-Fc (e.g., comprising amino acids 21-253 of SEQ ID NO: 8), a mouse IgG2b-Fc (e.g., comprising amino acids 21-259 of SEQ ID NO: 9), a mouse IgG3-Fc (e.g., comprising amino acids 21-253 of SEQ ID NO: 10), a canine IgG-A Fc (e.g., comprising amino acids 21-244 of SEQ ID NO: 15), or a feline IgG1 Fc (e.g., comprising amino acids 21-243 of SEQ ID NO: 17). Preferably, the species of the immunoglobulins is chosen to correspond with the species of the subject to whom the fusion protein will be administered.

In some embodiments, the peptides comprise an IgG antibody heavy chain CH2 region, an IgG antibody heavy chain CH3 region, and an enzymatic luminal domain or a catalytic domain of sialyltransferase. In some embodiments, the peptide has the amino acid sequence that is set forth in SEQ ID NO: 11. The disclosure also provides polypeptides comprising an IgG antibody heavy chain CH2 region, an IgG antibody heavy chain CH3 region, and an enzymatic luminal domains or a catalytic domain of galactosyltransferase. In some embodiments, the peptide has the amino acid sequence that is set forth in SEQ ID NO: 12. In some embodiments, these polypeptides can form a homodimer. The homodimer can have two enzymatic luminal domains (or catalytic domains) of sialyltransferase (e.g., FcST6 or ST6^{Fc}). In some other cases, the homodimer can have two enzymatic luminal domains (or catalytic domains) of galactosyltransferase (e.g., FcB4 or B4^{Fc}). In some embodiments, these polypeptides can form a heterodimer (FcChm or FcB4/FcST6), which has one enzymatic luminal domain (or catalytic domain) of sialyltransferase and one enzymatic luminal domain (or catalytic domain) of galactosyltransferase.

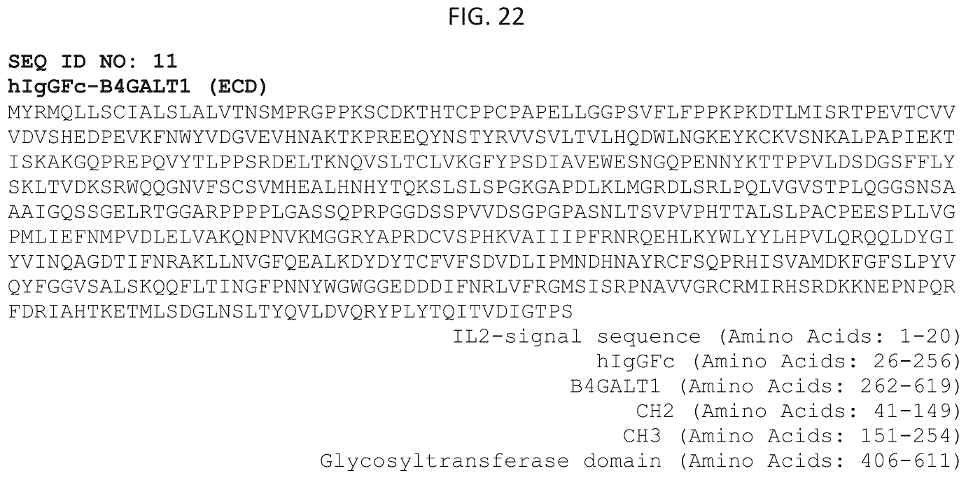

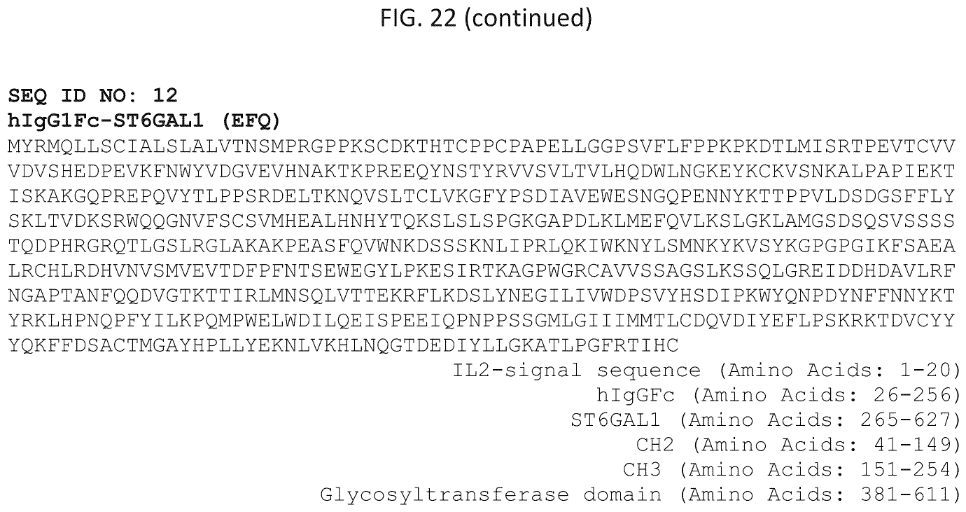

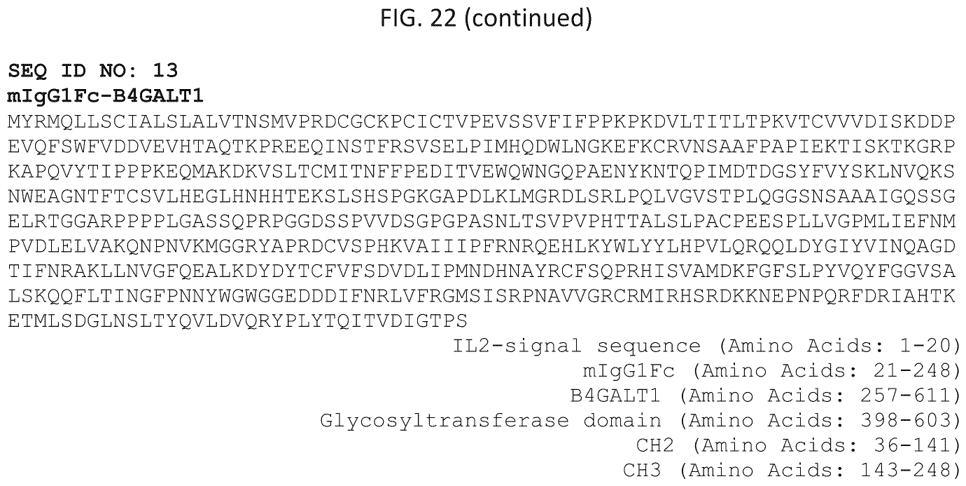

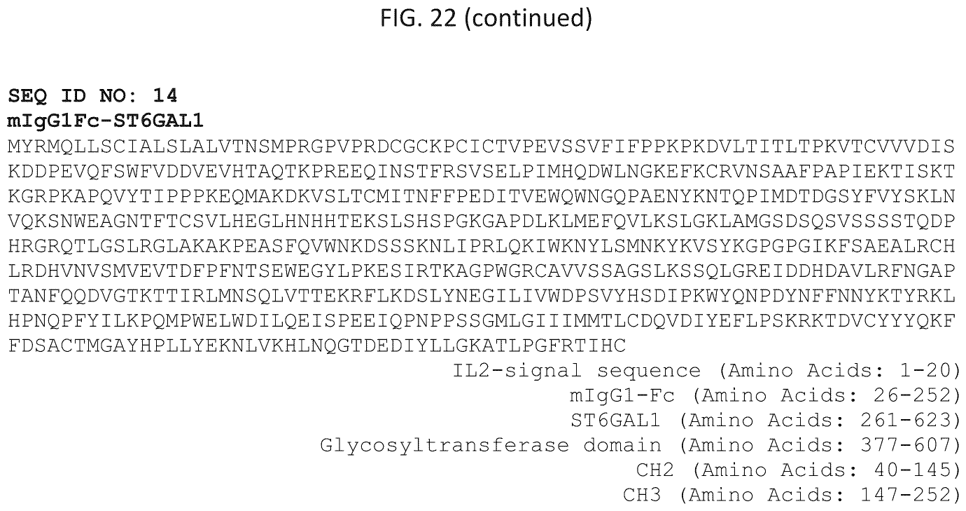

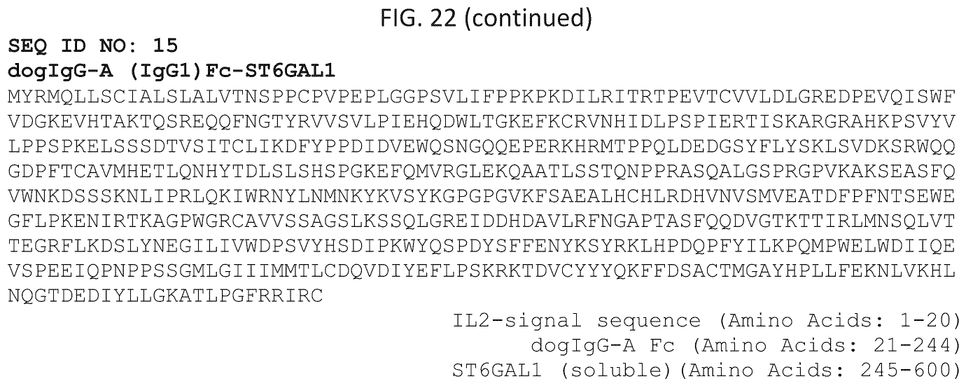

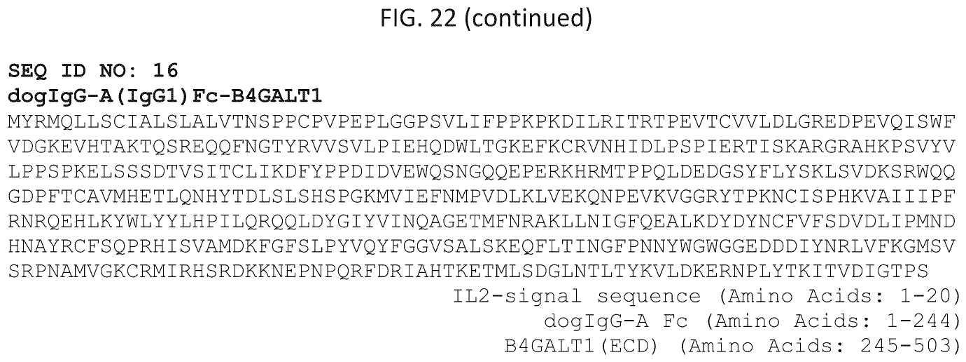

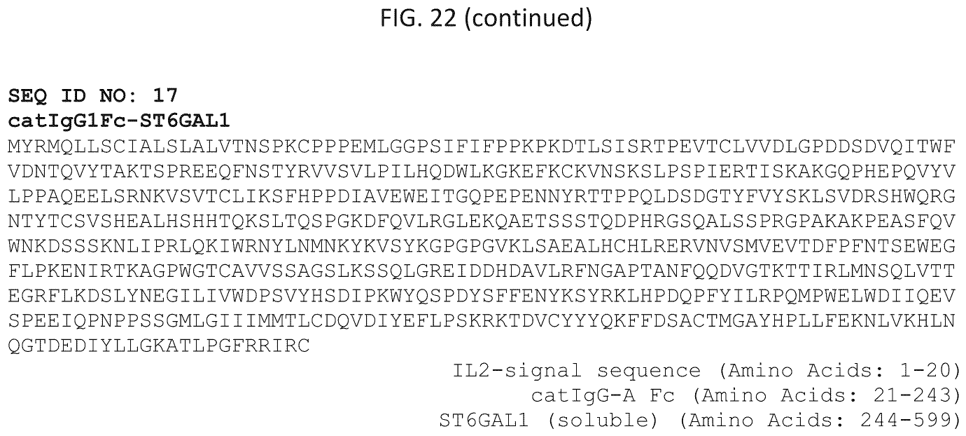

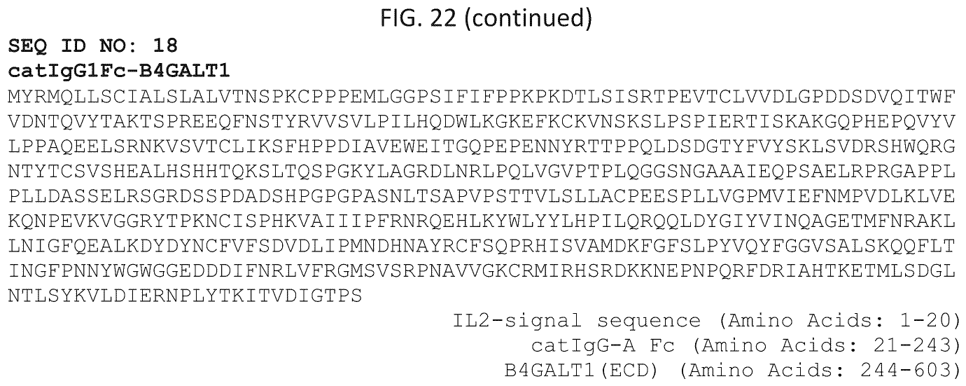

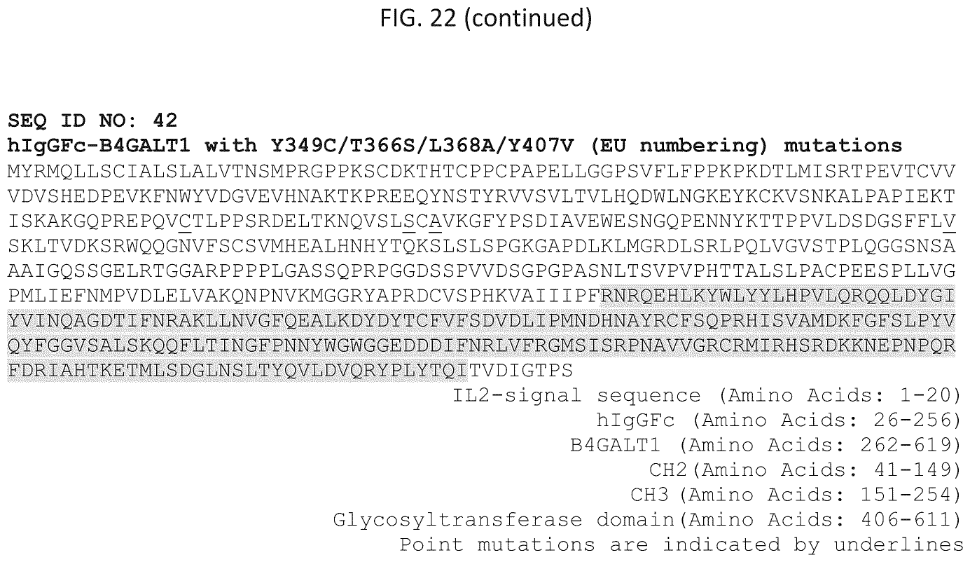

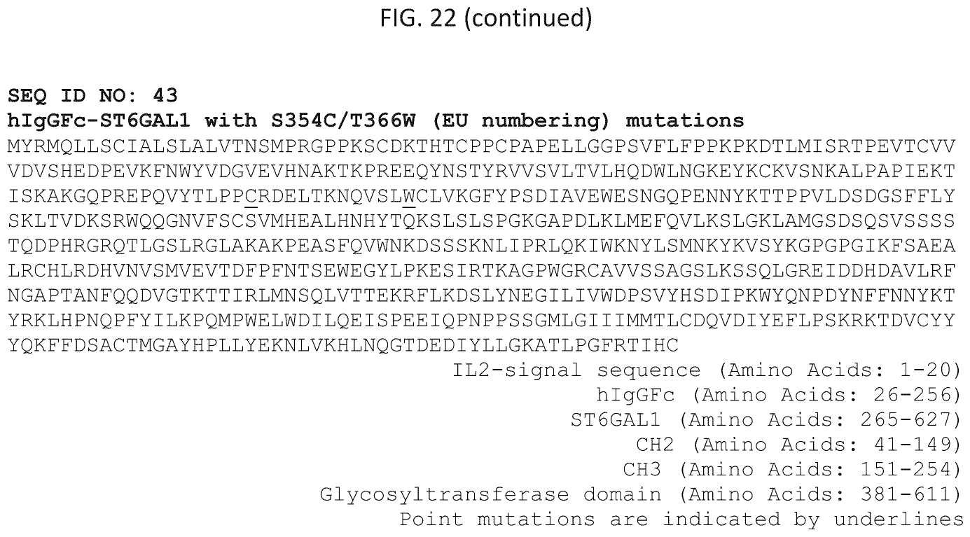

FIG. 22 shows some examples of glycosylation enzyme—Fc fusion proteins, including human IgG Fc-B4GALT1 fusion protein (SEQ ID NO: 11), human IgG1 Fc-ST6GAL1 fusion protein (SEQ ID NO: 12), mouse IgG1 Fc-B4GALT1 fusion protein (SEQ ID NO: 13), mouse IgG1 Fc-ST6GAL1 fusion protein (SEQ ID NO: 14), canine IgG-A (IgG1) Fc-ST6GAL1 fusion protein (SEQ ID NO: 15), canine IgG-A(IgG1) Fc-B4GALT1 fusion protein (SEQ ID NO: 16), feline IgG1 Fc-ST6GAL1 fusion protein (SEQ ID NO: 17), feline IgG1 Fc-B4GALT1 fusion protein (SEQ ID NO: 18).

In some embodiments, these peptides can additionally have signal sequences, e.g., IL2-signal sequence (amino acids 1-20 of SEQ ID NO: 3), or x-signal sequence (amino acids 1-39 of SEQ ID NO: 4). These signal sequences usually present at the N-terminus of the peptides.

In some embodiments, the Fc regions can have “knobs-into-holes” (KIH) mutations. The KIH mutations can be used for facilitating the formation of heterodimers. In some embodiments, one Fc has one or more of mutations selected from the group consisting of Y349C, T366S, L368A, and Y407V (all in EU numbering). The other Fc can have one or both mutations selected from the group consisting of S354C and T366W (all in EU numbering). In some embodiments, Fc-B4GALT1 fusion protein has a sequence set forth in SEQ ID NO: 42. In some embodiments, Fc-ST6GAL1 fusion protein has a sequence set forth in SEQ ID NO: 43.

In some embodiments, the enzymatic luminal domain, soluble domain or the catalytic domain of glycosylation enzymes can be fused to a part or the entire part of IgG1, IgG2, IgG3, or IgG4. In some embodiments, the enzymatic luminal domain, soluble domain or the catalytic domain of glycosylation enzymes can be fused to Fc portions of IgG1, IgG2, IgG3, or IgG4. In some embodiments, the Fc portion is the Fc of IgG3.

Intact antibodies with desired specificity can be fused to glycosylation enzymes, enabling specific targeting of the enzymes. Further, similar protein fusions can be generated using dog/cat/horse/cow equivalent/homologous antibodies or glycosylation enzymes, enabling treatment of non-human animals (e.g., pets and livestock).

### Monomers, Dimers, Trimers, and Tetramers

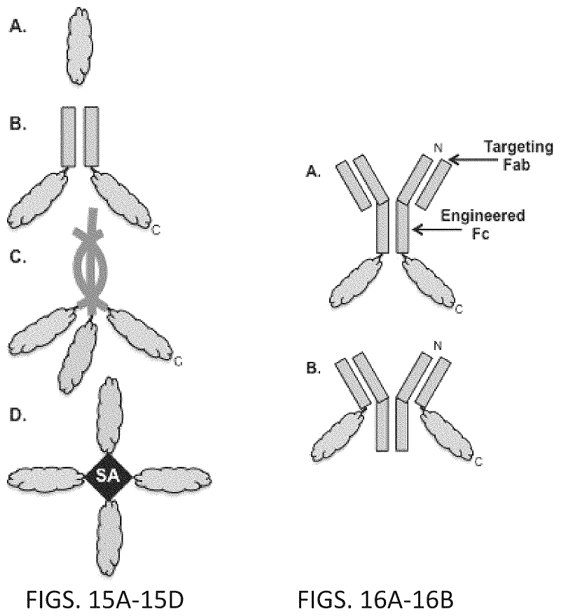

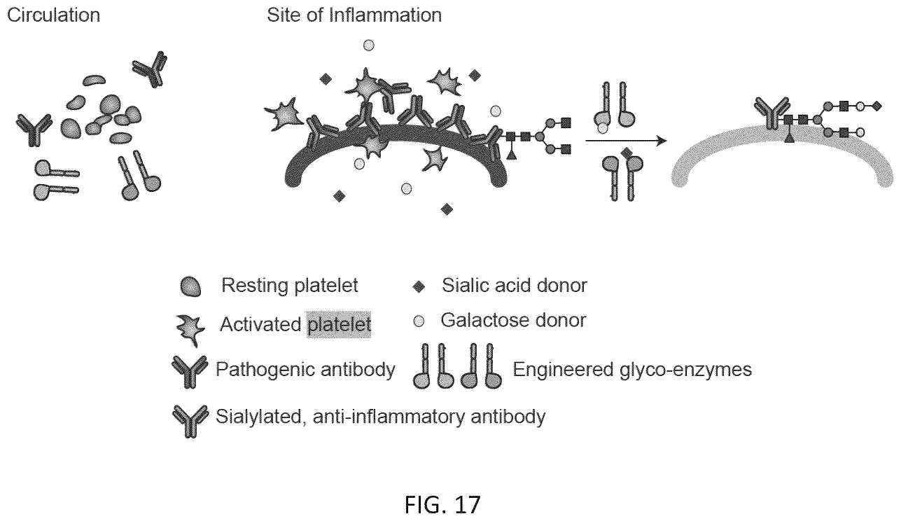

Exemplary monomers are shown in FIG. 15A. As shown in FIG. 15A, a monomer can be the enzymatic luminal domain, the soluble domain, or the catalytic domain of a glycosylation enzyme (e.g., sialyltransferase or galactosyltransferase).

Multimers can be generated by any methods known in the art. In some embodiments, the multimer can have one, two, three, four, or more than four enzymatic luminal domains, soluble domains, or catalytic domains of glycosylation enzymes. In some embodiments, the multimer can have one, two, three, four, or more than four enzymatic luminal domains (or catalytic domains) of sialyltransferase. In some embodiments, the multimer can have one, two, three, four, or more than four enzymatic luminal domains (or catalytic domains) of galactosyltransferase.

In some embodiments, the multimers can be dimers (e.g., FcB4, FcST6, and FcChm).

The multimers can also be trimers, or tetramers. Trimers, and tetramers can be generated by fusing the luminal enzymatic portion or the soluble portion to, e.g., a collagen-like peptide scaffold (Gly-Pro-Pro)×10, or biotinylation sites, respectively. As shown in FIGS. 15C-15D, soluble portions of glycosylation enzymes can be engineered as trimers by fusing to collagen trimerizing domains (FIG. 15C), or tetrameters by including biotin-ligase recognitions sites, biotinylating the fusion with biotin ligase, and subsequently incubating with streptavidin (FIG. 15D). In FIGS. 15C-15D, the multimers are drawn as heteromultimers, but can also be made as homomultimers, as needed. The detailed method of producing multivalent protein binders is described, e.g., in Fan, Chia-Yu, et al. “Production of multivalent protein binders using a self-trimerizing collagen-like peptide scaffold.” The FASEB Journal 22.11 (2008): 3795-3804, which is incorporated by reference in its entirety.

### Site-Specific Glycoengineering

Although soluble Golgi enzymes are found throughout the body, there are applications and circumstances where the ability to selectively target these enzymes to defined anatomical locations is more desirable. In some appropriate cases, the enzymatic luminal domain or the catalytic domain can be linked to immunoglobulin (e.g., antibody, or single-chain variable fragment) to yield full length antibody-enzyme conjugates.

A single native IgG antibody comprises two identical copies of a light chain and two identical copies of a heavy chain. The heavy chains, which each contain one variable domain (or variable region, VH) and three constant domains (or constant regions, CH1, CH2, CH3), bind to one another via disulfide bonding within their constant domains to form the “stem” of the antibody. The light chains, which each contain one variable domain (or variable region, VL) and one constant domain (or constant region, CL), each bind to one heavy chain via disulfide binding. The variable region of each light chain is aligned with the variable region of the heavy chain to which it is bound. The fragment antigen-binding (Fab) fragment is a region on an antibody that binds to antigens. It is composed of one constant and one variable region of each of the heavy and the light chain. The fragment crystallizable region (Fc region) is the tail region of an antibody that interacts with cell surface receptors called Fc receptors and some proteins of the complement system. This property allows antibodies to activate the immune system.

In IgG, the Fc region is composed of two identical protein fragments, derived from the second and third constant regions of the antibody's two heavy chains.

Fusion to the Fc or constant C-terminus of the Fab will depend on the specific application (FIGS. 16A-16B). Similarly, the variable region sequence used in the full-length immunoglobulin will be selected by the ability to localize the immunoglobulin-attached glycosylation enzymes. Criteria for variable region sequences will include recognition of a motif located adjacent to a glycosylation site of interest, such that a fused enzyme will be in close proximity to modify the target glycan. Also, variable region sequences that bind directly to the glycan or block the accessibility of the glycan will be avoided. Finally, the Fc portion can be engineered to elicit desired antibody-dependent effector functions (FIG. 16A). For example, for glycosylation enzymes that target infectious agents, Fcs that exhibit enhance antibody-dependent cytotoxicity (ADCC) can be effective. In contrast, glycosylation enzymes-fusions for other applications, including xenograft transplantation, would contain Fcs incapable of ADCC.

As shown in FIGS. 16A-16B, enzyme fusions can be generated using immunoglobulin variable chains and constant chains of interest. Enzymes can be fused to the C-terminus of the Fc or constant Fab, depending on the application. Fabs are selected for their specificity, ability to bring glycosylation enzyme near enough to the intended glycan. The Fc can be engineered to elicit, or not elicit, desired immunoglobulin effector functions.

In some embodiments, the disclosure provides a heteromultimer. The heteromultimer includes an antibody or antibody fragment thereof, an enzymatic luminal domain (or catalytic domain) of sialyltransferase, and/or an enzymatic luminal domain (or catalytic domain) of galactosyltransferase. In some embodiments, the antibody or antibody fragment thereof has two heavy chains, and two light chains. The enzymatic luminal domain (or catalytic domain) of sialyltransferase can be fused to the C-terminus of the heavy chain or the light chain. Similarly, the enzymatic luminal domain (or catalytic domain) of galactosyltransferase can also be fused to the C-terminus of the heavy chain or the light chain.

### Methods of Treatment

The methods described herein include methods for the treatment of IgG-mediated disorders (e.g., inflammation, autoimmune diseases) and antibody-mediated injury in organ transplantation. In some embodiments, the disorder is an autoimmune disease such as idiopathic thrombocytopenic purpura (ITP), multiple sclerosis, systemic lupus erythematosus, rheumatoid arthritis, chronic inflammatory demyelinating neuropathy, Sjogren's syndrome, and Granulomatosis with Polyangitis (Wegner's), etc. These autoimmune diseases are described, e.g., in Dal, Mehmet Sinan, et al. “Assessment of the underlying causes of the immune thrombocytopenia: Ten years experience.” JPMA. The Journal of the Pakistan Medical Association 67.7 (2017): 1004; Prineas, John W., and John D E Parratt. “Multiple sclerosis: Serum anti-CNS autoantibodies.” Multiple Sclerosis Journal (2017): 1352458517706037; Bai, Yungiang, et al. “Self—dsDNA in the pathogenesis of systemic lupus erythematosus.” Clinical & Experimental Immunology (2017); Hughes, Graham R V. “Frequency of anti-DNA antibodies in SLE, RA and other diseases: experience with the ammonium sulphate precipitation technique.” Scandinavian Journal of Rheumatology 4.sup11 (1975): 42-51; Fu, S. M., et al. “Autoantibodies and glomerulonephritis in systemic lupus erythematosus.” Lupus 12.3 (2003): 175-180; Tan, Eng M. “Autoantibodies and autoimmunity: A three-decade perspective. A tribute to Henry G. Kunkel.” Annals of the New York Academy of Sciences 815.1 (1997): 1-14; Querol et al., “Autoantibodies in chronic inflammatory neuropathies: diagnostic and therapeutic implications.” Nat Rev Neurol. 2017 Sep.; 13(9):533-547; each of which is incorporated by reference herein in its entirety.

Generally, the methods include administering a therapeutically effective amount of agents (e.g., fusion proteins or peptides) comprising or consisting of the enzymatic luminal domain or the catalytic domain of glycosylation enzymes, multimers (e.g., FcB4, FcST6, FcChm), or compositions as described herein, to a subject who is in need of, or who has been determined to be in need of, such treatment.

As used in this context, to “treat” means to ameliorate at least one symptom of the disorders or the diseases. Often, the treatment results in an improvement in the symptoms. In some embodiments, the treatment can result in a reduction of inflammation. In some embodiments, one or more of the clinical symptoms are ameliorated or reduced, the duration is shortened, the frequency of the occurrence of the symptoms is reduced, or the clinical symptoms are prevented from manifesting (i.e., the risk of the symptoms is reduced).

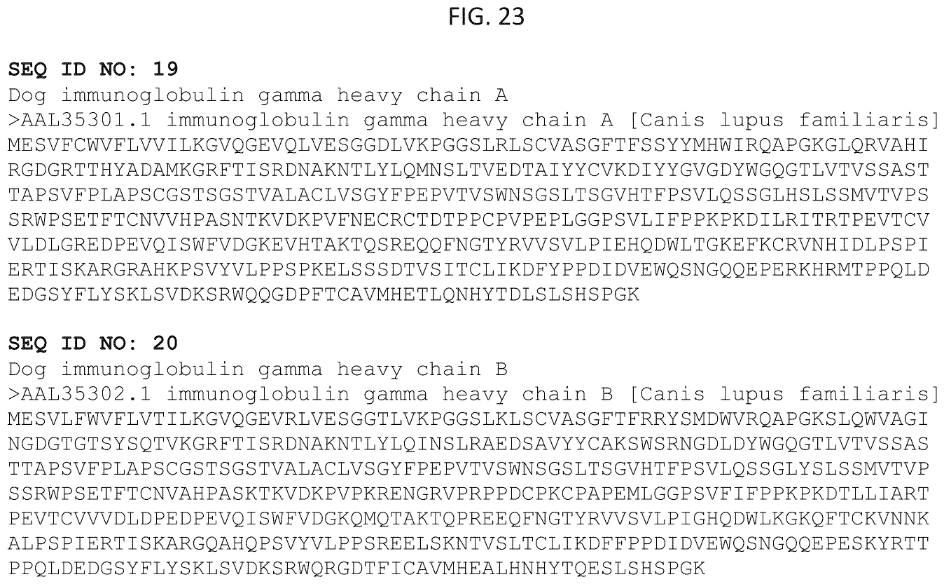

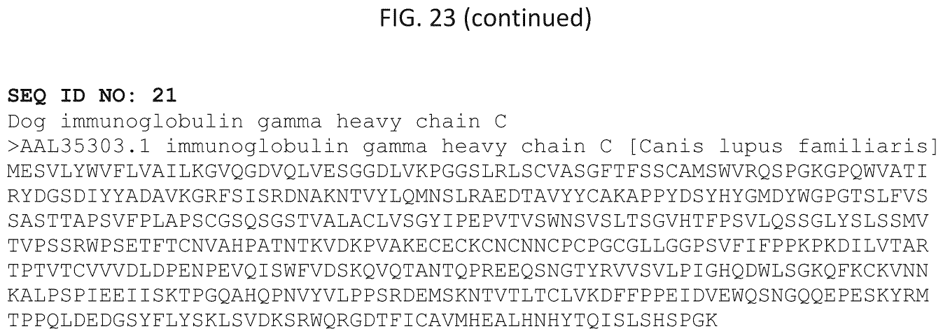

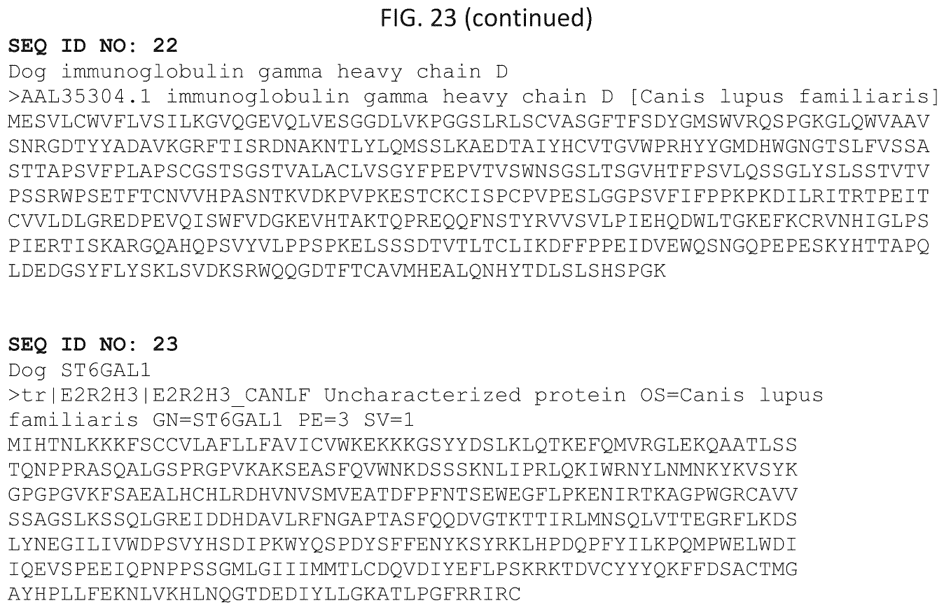

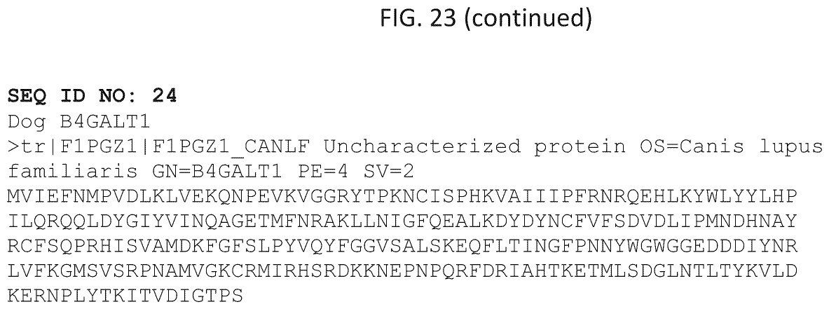

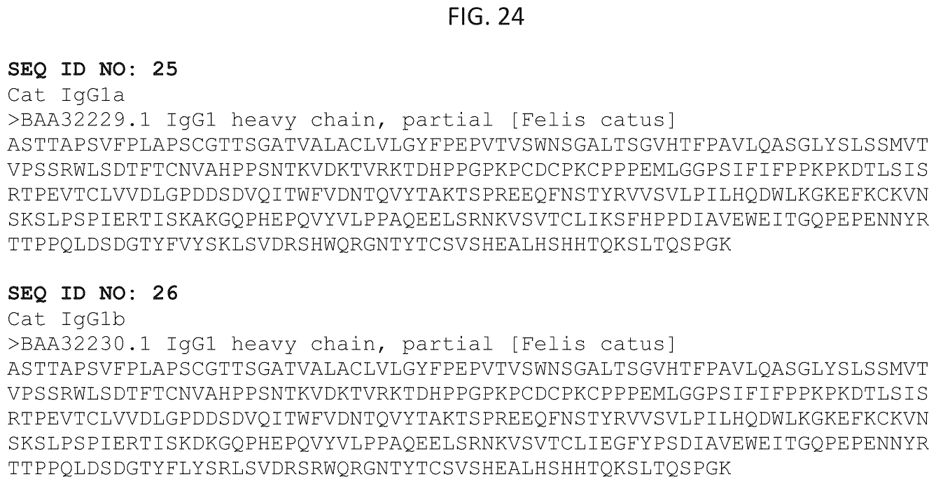

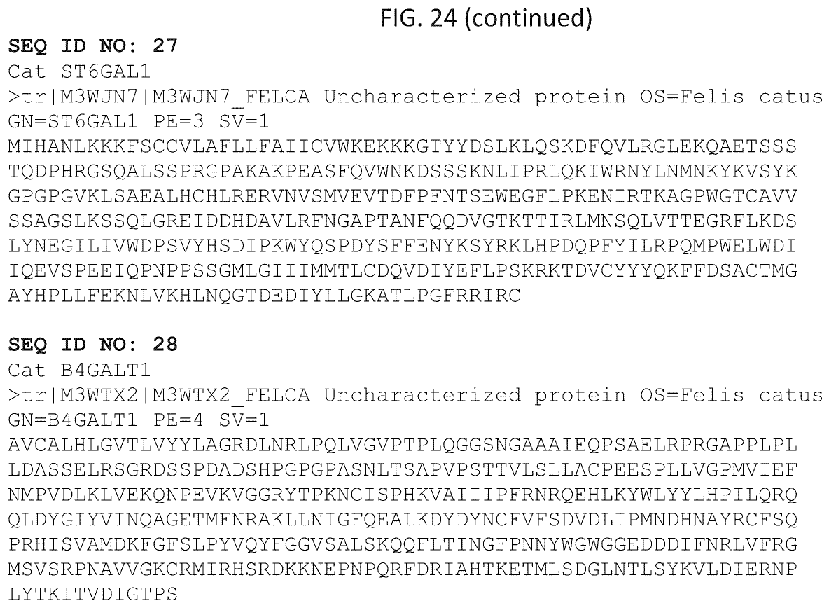

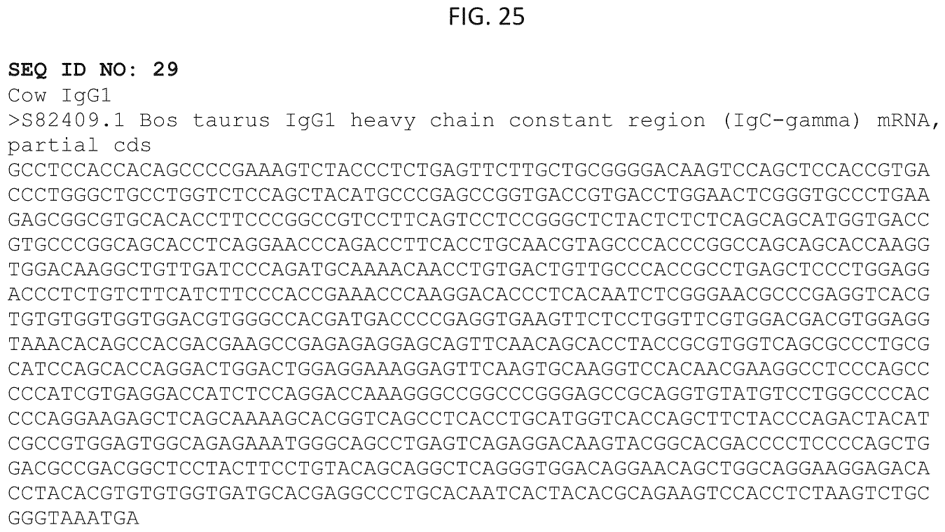

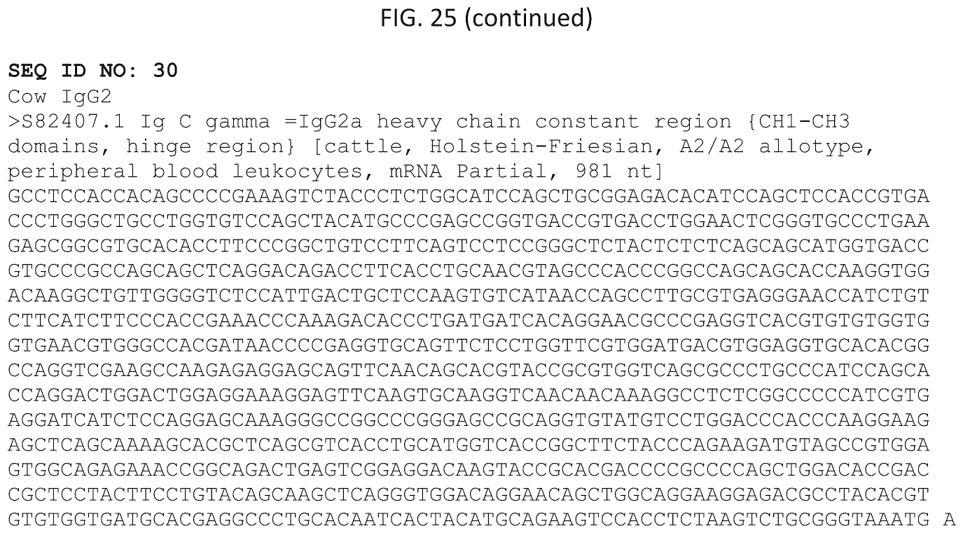

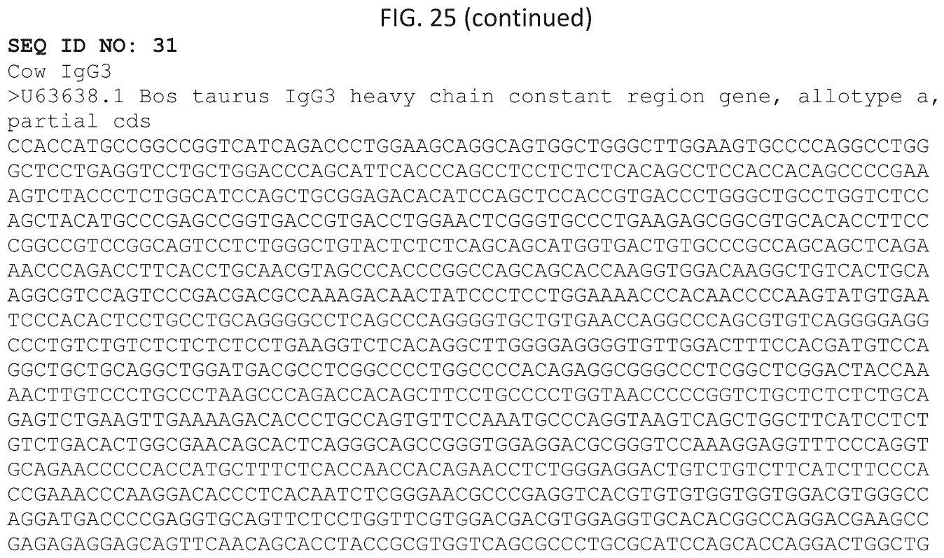

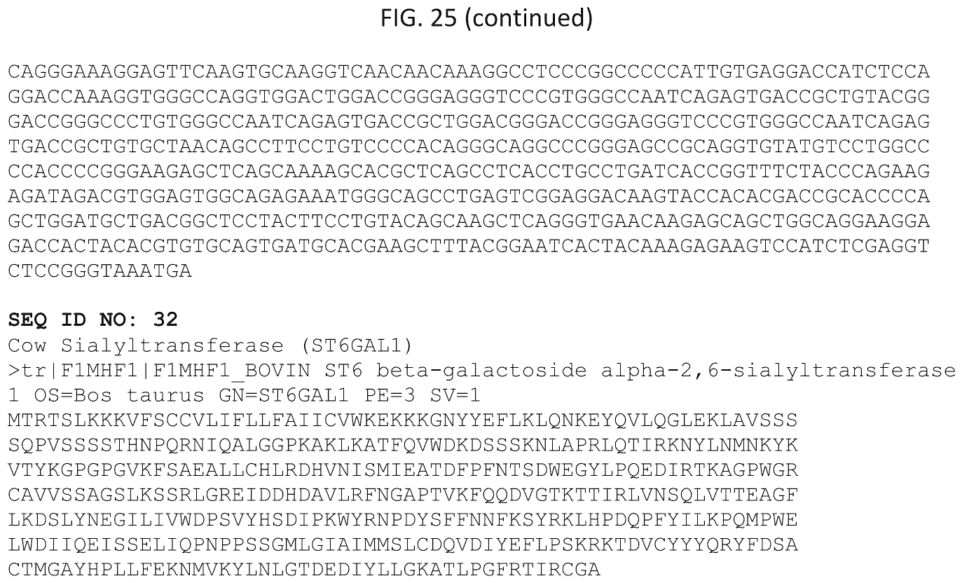

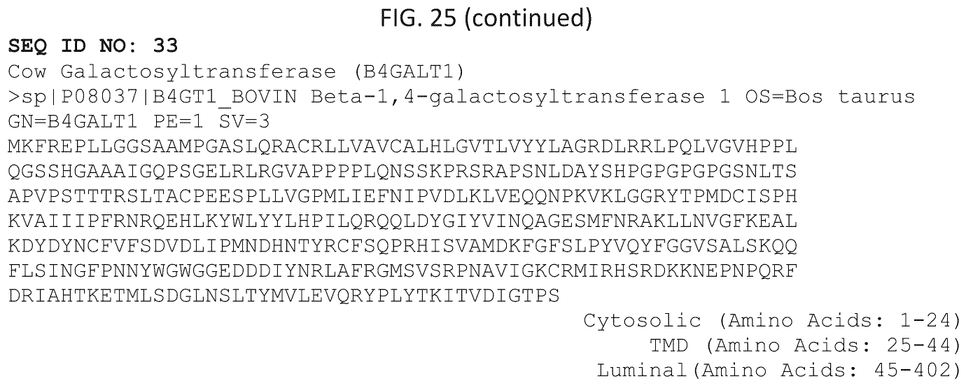

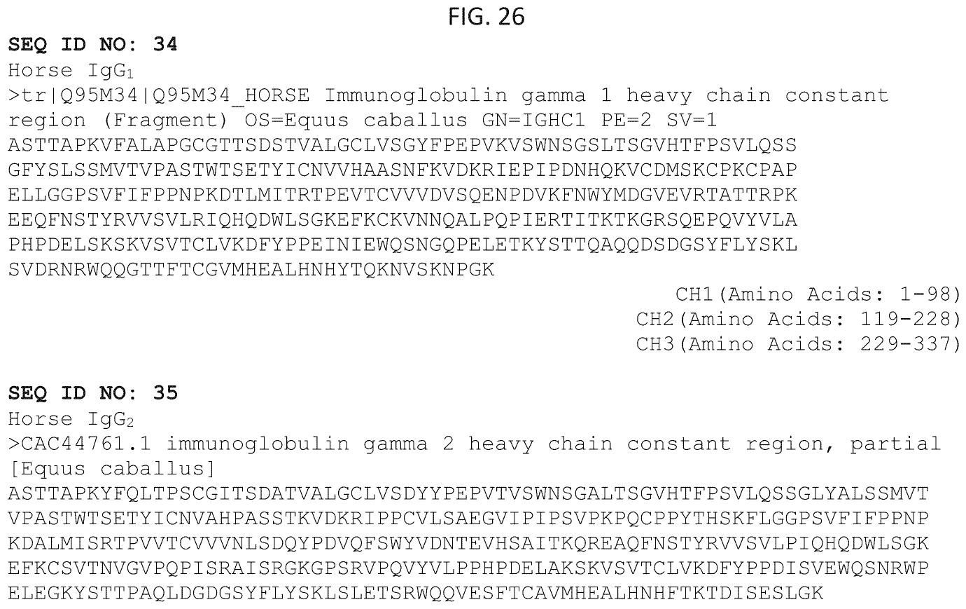

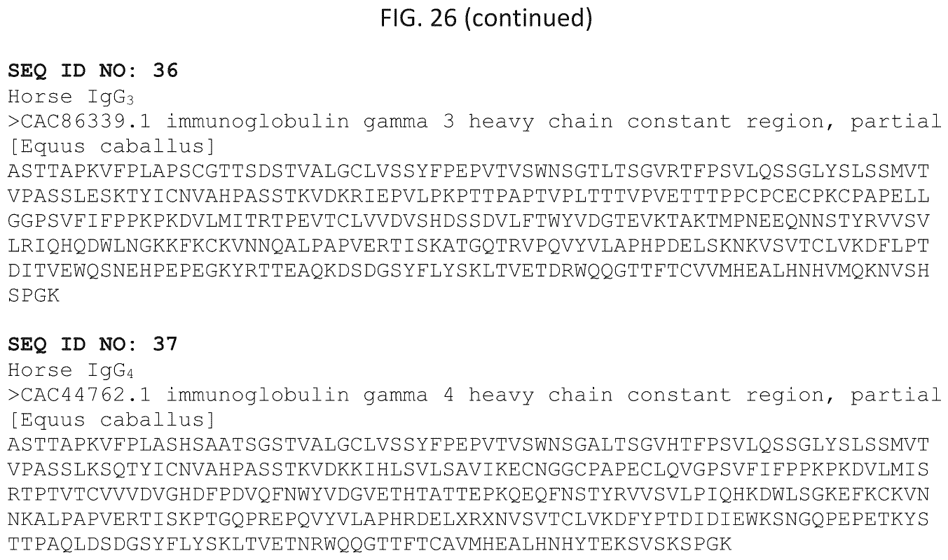

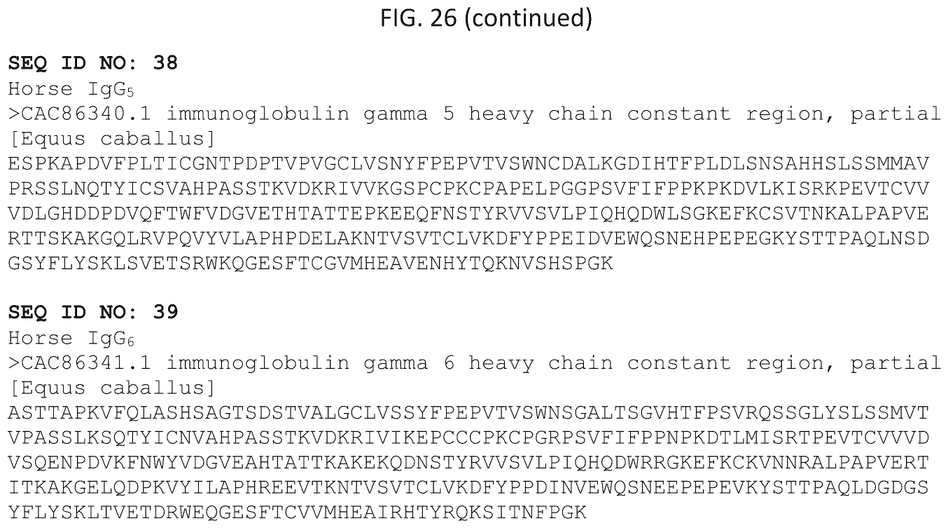

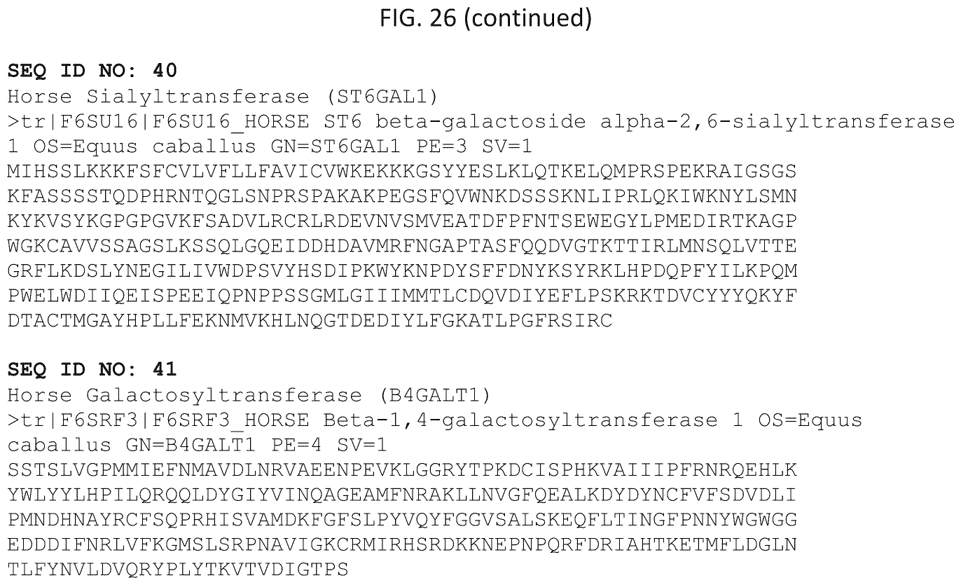

As used herein, the terms “subject” and “patient” are used interchangeably throughout the specification and describe an animal, human or non-human, e.g., a mammal, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. Thus, the glycosylation enzymes, the antibodies, or the parts thereof (e.g., Fc regions of the antibodies or the catalytic domain of the glycosylation enzymes) as described herein can also derive from these non-human animals. The present disclosure further provides the amino acid sequences of the glycosylation enzymes, and the antibodies or the parts thereof that derive from some of these non human animals. For example, FIG. 23 lists the amino acid sequences of dog IgG heavy chain A, dog IgG heavy chain B, dog IgG heavy chain C, dog IgG heavy chain D, dog ST6GAL1, and dog B4GALT1. FIG. 24 lists the amino acid sequences of cat IgG1a heavy chain, cat IgG1b heavy chain, cat ST6GAL1, and cat B4GALT1. FIG. 25 lists the amino acid sequences of cow IgG1 heavy chain constant region, cow IgG2 heavy chain constant region, cow IgG3 heavy chain constant region, cow ST6GAL1, and cow B4GALT1. FIG. 26 lists the amino acid sequences of horse IgG1 heavy chain constant region, horse IgG2 heavy chain constant region, horse IgG3 heavy chain constant region, horse IgG4 heavy chain constant region, horse IgG5 heavy chain constant region, horse IgG6 heavy chain constant region, horse ST6GAL1, and horse B4GALT1.

In some embodiments, the subject is a human (e.g., male human or female human) with an age over 25 years old, 30 years old, 40 years old, 50 years old, 60 years old, 70 years old, or 80 years old.

As used herein, the terms “therapeutically effective” and “effective amount”, used interchangeably, applied to a dose or amount refers to a quantity of a composition, compound or pharmaceutical formulation that is sufficient to result in a desired activity upon administration to a subject in need thereof. Within the context of the present disclosure, the term “therapeutically effective” refers to that the composition, compound or pharmaceutical formulation, in a sufficient amount, can reduce or eliminate at least one symptom or one condition of the disorders as described herein.

### IgG-Mediated Disorder

As used herein, the term “IgG-mediated disorder” refers to any disorder caused by or characterized by an increased level or an increased activity of IgG. Therefore, inhibiting the activity of IgG is often the treatment for a IgG-mediated disorder. The IgG-mediated disorder can include, but are not limited to, inflammation, and various auto-immune diseases.

In fact, IgG is widely used in the clinic to suppress inflammation. Intravenous immunoglobulin (IVIG) is a therapeutic preparation of polyclonal, monomeric IgG derived from tens of thousands of healthy donors. It is given to immunocompromised patients at 400-600 mg/kg as an antibody replacement therapy. In 1981, IVIG was administered at a high dose (1-2 g/kg) to pediatric patients suffering from an autoimmune disease in which autoantibodies target platelets (immune-mediated thrombocytopenia, ITP). The treatment restored platelet counts to normal, offering temporary relief to the patients (Imbach, P. et al. Lancet 1, 1228-1231 (1981)). Since then, IVIG is routinely used as an anti-inflammatory therapy for the treatment of many diseases, including the autoimmune diseases ITP, multiple sclerosis, systemic lupus erythematosus, and for solid organ transplantation (see, e.g., Fillit, H. Lancet Neurol 3, 704 (2004); Fillit, H., Hess, G., Hill, J., Bonnet, P. & Toso, C. Neurology 73, 180-185 (2009); Hack, C. E. & Scheltens, P. J Neurol Neurosurg Psychiatry 75, 1374-1375 (2004); Ishii, N., Hashimoto, T., Zillikens, D. & Ludwig, R. J. Clin Rev Allergy Immunol 38, 186-195 (2010); Nimmerjahn, F. & Ravetch, J. V. Annu Rev Immunol 26, 513-533 (2008)).

The mechanisms governing the dose-dependent pro- and anti-inflammatory actions of IgG have been extensively studied (Clynes, R. Curr Opin Immunol 19, 646-651 (2007); Schwab, I. & Nimmerjahn, F. Nat Rev Immunol 13, 176-189 (2013)). Removal of the Fc glycan from IgG has shed light on the mechanism driving the anti-inflammatory activity of IgG (Kaneko, Y., Nimmerjahn, F. & Ravetch, J. V. Science 313, 670-673 (2006)). De-glycosylated IVIG was unable to suppress inflammation in a model of rheumatoid arthritis. Further, IVIG treated with neuraminidase, to removed terminal sialic acid from the Fc glycan, also exhibited no anti-inflammatory activity. Thus, the IgG Fc glycan composition, and specifically terminal sialic acid, is responsible for the anti-inflammatory activity of IgG. Further, sialylated IgG Fc exhibited anti-inflammatory activity at a 30-fold lower dose than IVIG.

Therefore, the methods described in this disclosure can be used to treat various IgG-mediated disorder (e.g., inflammation, autoimmune diseases).

In another aspect, the methods described in this disclosure can be used to treat any diseases or disorders that can be treated by IVIG. These disease or disorders include, but not limited to, inflammation, autoimmune diseases, idiopathic thrombocytopenic purpura (ITP), multiple sclerosis, systemic lupus erythematosus, and Alzheimer's Disease.

In some embodiments, the methods include the steps of identifying a subject having an IgG-mediated disorder, and administering to the subject any polypeptides, multimers, or compositions as described in this disclosure to the subject.

### Inflammation

Inflammation is part of the complex biological response of body tissues to harmful stimuli. The function of inflammation is to eliminate the initial cause of cell injury, clear out necrotic cells and tissues damaged from the original insult and the inflammatory process, and to initiate tissue repair. However, in some cases, inflammation can cause harm to the body. As discussed above, inflammation is often mediated by antibodies. Therefore, the methods described herein can be used to treat, inhibit, reduce, or control inflammation.

### Autoimmune Diseases

An autoimmune disease is a condition arising from an abnormal immune response to a normal body part. As auto-immune diseases are often mediated by abnormal function of IgG, thus the methods described in this disclosure can be used to treat various autoimmune diseases. The autoimmune diseases can affect major organ (e.g., heart, kidney, liver, lung, skin, and reproductive organs), gland (e.g., adrenal gland, pancreas, thyroid gland, and salivary gland), digestive system, blood, connective tissue, muscle, nervous system, eye, ear, vascular system, etc. Some common autoimmune diseases include celiac disease, diabetes mellitus type 1, Graves disease, inflammatory bowel disease, multiple sclerosis, psoriasis, rheumatoid arthritis, and systemic lupus erythematosus.

A list of autoimmune diseases that can be treated by the methods described in this disclosure includes, but are not limited to, Addison's disease, agammaglobulinemia, alopecia areata, amyloidosis, ankylosing spondylitis, anti-gbm/anti-tbm nephritis, antiphospholipid syndrome, autoimmune hepatitis, autoimmune inner ear disease, axonal & neuronal neuropathy, Behcet's disease, bullous pemphigoid, Castleman disease, celiac disease, chagas disease, chronic inflammatory demyelinating polyneuropathy, chronic recurrent multifocal osteomyelitis, Churg-Strauss, cicatricial pemphigoid/benign mucosal pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, systemic lupus erythematosus, Dressler's syndrome, endometriosis, eosinophilic esophagitis (EoE), eosinophilic fasciitis, erythema nodosum, essential mixed cryoglobulinemia, evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves disease, Guillain-Barre syndrome, Hashimoto's thyroiditis, hemolytic anemia, Henoch-Schonlein purpura, herpes gestationis or pemphigoid gestationis, hypogammalglobulinemia, IgA nephropathy, IgG4-related sclerosing disease, inclusion body myositis, interstitial cystitis, juvenile arthritis, juvenile diabetes (Type 1 diabetes), juvenile myositis, kawasaki disease, lambert-eaton syndrome, leukocytoclastic vasculitis, lichen planus, lichen sclerosus, ligneous conjunctivitis, linear IgA disease, lupus, lyme disease chronic, Meniere's disease, microscopic polyangiitis, mixed connective tissue disease, Mooren's ulcer, Mucha-Habermann disease, multiple sclerosis, myasthenia gravis, myositis, narcolepsy, neuromyelitis optica, neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria, Parry Romberg syndrome, pars planitis (peripheral uveitis), Parsonnage-Turner syndrome, pemphigus, peripheral neuropathy, perivenous encephalomyelitis, pernicious anemia, POEMS syndrome (polyneuropathy, organomegaly, endocrinopathy, monoclonal gammopathy, skin changes), polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, Rheumatoid Arthritis, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis, Susac's syndrome, sympathetic ophthalmia, Takayasu's arteritis, temporal arteritis/giant cell arteritis, thrombocytopenic purpura (e.g., idiopathic thrombocytopenic purpura), Tolosa-Hunt syndrome, transverse myelitis, type 1 diabetes, ulcerative colitis, undifferentiated connective tissue disease, uveitis, vasculitis, vitiligo, Wegener's granulomatosis (now termed Granulomatosis with Polyangiitis).

In some cases, the autoimmune disease is an acquired autoimmune disorder. For example, infection with HIV can cause destruction of the immune system leading to damage to several organ systems and tissues. Thus, in one aspect, the methods described in this disclosure can be used to treat an acquired autoimmune disorder.

### Solid Organ Transplantation

Transplant rejection occurs when transplanted tissue is rejected by the recipient's immune system, which destroys the transplanted tissue. The transplant rejection often involves antibody-mediated injury. The role of antibodies in transplantation is described, e.g., in Hourmant et al. “Frequency and clinical implications of development of donor-specific and non-donor-specific HLA antibodies after kidney transplantation.” Journal of the American Society of Nephrology 16.9 (2005): 2804-2812, which is incorporated by reference in its entirety. Thus, in one aspect, the methods described in this disclosure can be used to treat or control transplant rejection (e.g., reduce or minimize antibody-mediated injury), or treat graft-versus-host disease. In some embodiments, the transplanted organ is heart, kidneys, liver, lungs, pancreas, intestine, skin, or thymus.

### Vaccination Improvement

Vaccines can stimulate the immune system to generate antibodies that are specific for pathogen antigens. Studies have demonstrated that sialylated antibodies specific for vaccine antigens, through a feed-forward mechanism, result in generation of highly-specific and high affinity antibodies. This mechanism is described, e.g., in Wang, Taia T., et al. “Anti-HA glycoforms drive B cell affinity selection and determine influenza vaccine efficacy.” Cell 162.1 (2015): 160-169. Thus, the methods described herein can also be used in conjunction with standard vaccinations to generate sialylated antibodies specific for vaccine antigens, ultimately yielding highly-specific and high affinity antibodies for pathogen antigens.

In some embodiments, the methods include administering a therapeutically effective amount of polypeptides, multimers (e.g., FcB4, FcST6, and/or FcChm), or compositions as described herein, to a subject, before, during, or after the subject is administered with vaccines. In some embodiments, a composition comprising a vaccine and the polypeptides, multimers (e.g., FcB4, FcST6, and/or FcChm), or compositions as described herein is administered to the subject.

### Expression Systems

To use the fusion proteins or peptides as described herein, it may be desirable to express them from a nucleic acid that encodes them. This can be performed in a variety of ways. For example, the nucleic acid encoding the fusion proteins or peptides can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion proteins or peptides for production. The nucleic acid encoding the fusion proteins or peptides can also be cloned into an expression vector, for administration to a plant cell, animal cell, preferably a mammalian cell or a human cell, fungal cell, bacterial cell, or protozoan cell.

To obtain expression, a sequence encoding a fusion protein or peptide is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available. In some embodiments, the fusion proteins and peptides are expressed by transfection of HEK-293T cells, Expi293 cells, or CHO cells with vectors comprising the polynucleotides encoding fusion proteins and peptides as described in this disclosure.

The promoter used to direct expression of a nucleic acid depends on the particular application. For example, a strong constitutive promoter is typically used for expression and purification of fusion proteins. In contrast, when a vector encoding the fusion protein or peptide is to be administered in vivo, either a constitutive or an inducible promoter can be used, depending on the particular need. In some embodiments, the promoter for administration of the vector encoding the fusion protein or peptide can be a weak promoter, such as HSV TK or a promoter having similar activity. The promoter can also include elements that are responsive to trans activation, e.g., hypoxia response elements, Ga14 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein or peptide, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the fusion protein or peptide can include RNA Pol III promoters to drive expression of the guide RNAs, e.g., the H1, U6 or 7SK promoters. These human promoters allow for expression of fusion protein or peptide in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the fusion protein or peptide.

The present disclosure also includes the vectors and cells comprising the vectors, as well as kits comprising the proteins and nucleic acids described herein, e.g., for use in various methods as described herein.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of polypeptides, multimers (e.g., FcB4, FcST6, FcChm), or compositions (i.e., an effective dosage) depends on the polypeptides, multimers, or compositions that are selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the polypeptides, multimers, or compositions described herein can include a single treatment or a series of treatments.

Dosage, toxicity and therapeutic efficacy of the polypeptides, multimers, or compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Polypeptides, multimers, or compositions which exhibit high therapeutic indices are preferred. While polypeptides, multimers, or compositions that exhibit toxic side effects may be used, care should be taken to design a delivery system that targets polypeptides, multimers, or compositions to the site of affected tissue in order to minimize potential damage to uninfected cells and, thereby, reduce side effects.

The data obtained from cell culture assays and animal studies can be used in formulating a range of dosage for use in humans. The dosage of polypeptides, multimers, or compositions lies preferably within a range of circulating concentrations that include the ED50 with little or no toxicity. The dosage may vary within this range depending upon the dosage form employed and the route of administration utilized. For any polypeptides, multimers, or compositions used in the methods as described in this disclosure, the therapeutically effective dose can be estimated initially from cell culture assays. A dose may be formulated in animal models to achieve a circulating plasma concentration range that includes the IC50 (i.e., the concentration of the test polypeptide, multimer, or composition which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the use of pharmaceutical compositions comprising any polypeptides or multimers (e.g., FcB4, FcST6, and/or FcChm) as described in this disclosure as an active ingredient, as well as the compositions themselves. In some embodiments, the composition comprises FcB4, FcST6, and FcChm.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating polypeptides, multimers, or compositions as described in this disclosure in the required amount in an appropriate solvent with one or a combination of ingredients enumerated above, as required, followed by filtered sterilization. Generally, dispersions are prepared by incorporating the polypeptides, multimers, or compositions into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active agents can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the composition can be delivered in the form of an aerosol spray from a pressured container or dispenser that contains a suitable propellant, e.g., a gas such as carbon dioxide, or a nebulizer. Such methods include those described in U.S. Pat. No. 6,468,798.

In some embodiments, the polypeptides or multimers are prepared with carriers that will protect the polypeptides or multimers against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1: In Vivo Sialylation of IgG Antibodies Attenuates Autoimmune Disease

Experiments were performed to determine therapeutic effects of modulating IgG-mediated inflammation through glycoengineering of endogenous IgG in vivo.

### Materials and Methods

Construction and production of the glycosylation enzyme-Fc fusions. Human IgG Fc, preceded by IL2 secretion signal sequence, and soluble domains of ST6GAL1 (Beta-galactoside alpha-2,6 sialyltransferase 1) or B4GALT1 (Beta-1,4-galactosyltransferase 1) were joined by overlapping PCR, such that human IgG Fc is fused to 5′ end of the enzymes. A list of the primers used in this study is provided in Table 2. Restriction sites for HindIII (AAGCTT), XhoI (CTCGAG) were indicated in the table. The Fc-enzyme fusion genes were then TOPO cloned into a mammalian expression vector, pcDNA3.4, according to the manufacturer's protocol (Life Technologies). Recombinant Fc-enzymes were generated by transient transfection of the plasmids to Expi293 cells using Expi293 Expression System Kit (Life Technologies) according to the manufacturer's protocol. B4ST6^{Fe} enzyme was produced by co-transfecting pcDNA3.4/ST6^{Fe} and pcDNA3.4/B4^{Fe} at a 1:1 ratio. The enzymes were purified from the culture supernatant using Protein G agarose beads (Thermo Scientific) and dialyzed in PBS for in vivo injections.

In vivo Animal Studies. 7-8 weeks old C57BL/6 and NOD mice were purchased from the Jackson Laboratory and maintained in the animal facility at Massachusetts General Hospital (MGH) under specific pathogen free conditions according to the National Institutes of Health (NIH) guidelines. KRN TCR transgenic mice on a C57BL/6 background (K/B) were gifts from D. Mathis and C. Benoist (Harvard Medical School, Boston, MA) and were bred to NOD mice to generate K/B×N mice (Korganow et al., 1999). K/B×N serum was prepared as described previously (Kaneko et al., 2006). Inflammatory arthritis was induced by intravenous injection of K/B×N sera (200 μL of pooled K/B×N serum per mouse). For therapeutic intervention experiments, IVIG (1 g/kg), B4^{Fe} (1.25 mg/kg or 2.5 mg/kg), ST6^{Fc} (1.25 mg/kg or 2.5 mg/kg), B4ST6^{Fc} (2.5 mg/kg), or saline was injected day 0 or day 3 after K/B×N serum. Arthritis was scored by clinical examination, and the index of all four paws was added (0=unaffected, 1=swelling of one joint, 2=swelling of more than one joint, 3=severe swelling of the entire paw) as described (Kaneko et al., 2006). PBS, IVIG (1 g/kg) or B4ST6^{Fe} (50 μg) was injected 1 hr. prior to K/B×N serum injection. For nephrotoxic nephritis experiments, mice were pre-immunized with 200 μg of sheep IgG (BioRad) in CFA via intraperitoneal route, followed by intravenous injection of sheep NTS (Probetex, Inc.) (2 μl of serum per gram of mouse) 4 days later. IVIG (1 g/kg), B4ST6^{Fe} (50 μg) or its vehicle alone was injected 1 hour before sheep NTS injection. Urea nitrogen (BUN) in sera was measured by the enzyme coupled equilibrium method using a modified urease kit (Stanbio Laboratory). All animal experiments were conducted in compliance with the Institutional Animal Care and Use Committee of MGH.

Clopidogrel Treatment. Platelet inhibition was performed by daily injections of 10 mg/kg clopidogrel (Selleckchem) as previously described (Pucci et al., 2016). Treatment was initiated 2 days before K/B×N sera in the inflammatory arthritis model. In the nephrotoxic nephritis model, treatment was initiated 2 days after pre-immunization with sheep IgG. Treatment was continued for duration of 3 weeks.

In vitro glycosylation. Enzymatic activity of fusion enzymes was examined in vitro as previously described (Anthony et al., 2008). Briefly, glycan-acceptor protein (fetuin, human or mouse IgG Fc) was treated with Sialidase A (ProZyme) and β1,4-galactosidase-S(New England Biolabs, Inc.) overnight at 37° C. to remove sialic acid and galactose. To assess the galactosyltransferase activity of B4^{Fe} or B4ST6^{Fe}, asialylated, agalactosylated glycan-acceptor protein was incubated with 5 mM UDP-galactose (Calbiochem) in 2× galactosylation buffer (50 mM MOPS, 20 mM MnCl₂, pH7.2) overnight at 37° C. To assess the sialyltransferase activity of ST6^{Fe} or B4ST6^{Fe}, asialylated glycoprotein was incubated with 5 mM CMP-sialic acid (Nacalai tesque) in the sialylation buffer (150 mM NaCl, 20 mM HEPES, pH7.4) overnight at 37° C.

Western and Lectin Blots. Western and lectin blots were performed as described previously (Anthony et al., 2008). Briefly, equal amounts of protein were resolved on 4-12% Bis-Tris SDS-PAGE gel (Life Technologies) and then transferred to polyvinylidene difluoride membranes. After blocking the membranes with 5% dry milk in PBST (0.05% Tween 20) for western blot, proteins were detected using either anti-human IgG-HRP (20 ng/ml, Promega); anti-human B4GALT1 (100 ng/ml, Sigma-Aldrich) followed by anti-rabbit IgG-HRP (song/ml, Promega); or anti-human ST6GAL1 sera (1:100, generous gift from Dr. J. Paulson) followed by anti-rabbit IgG-HRP. For lectin blots, the membranes were blocked in Protein Free Blocking Buffer (Thermo Fisher Scientific), and probed with either biotinylated Sambucus Nigra Lectin (SNA; 5 μg/ml, Vector Laboratories) or with biotinylated Erythrina Cristagalli Lectin (ECL; 5 μg/ml, Vector Laboratories) to detect terminal sialic acid or galactose, respectively.

HPLC Glycan Analysis. Total or Fc specific N-linked glycan was released from glycoproteins using PNGaseF or Endo S (New England Biolabs, Inc.), respectively, according to manufacturer's instruction. Deglycosylation reactions were carried out at 37° C. overnight to ensure effective release of glycans. Glycans were purified from the reaction using GlykoClean™ G Cartridges (Prozyme), dried, and fluorescently labeled with 2-AB (2-aminobenzamide) (Sigma-Aldrich). Labeled glycans were cleaned with GlykoClean™ S-plus Cartridges (Prozyme), dried, and subjected to HPLC analysis. Glycan samples were dissolved in 100 mM ammonium formate (pH4.5) and separated using Agilent 1260 Infinity Quaternary LC system, outfitted with AdvanceBio Glycan Mapping column 2.1×150 mm, 2.7 μm and a fluorescent detector. Resulting peaks were analyzed in OpenLAB software (Agilent) and assigned glycoforms by comparing peaks of commercially available human IgG N-linked glycan library.

Measurement of sheep IgG-specific circulating IgG levels. 96-well ELISA plates coated with 5 μg/mL of sheep IgG were incubated with 1:500 diluted sera after blocking with 5% bovine serum albumin. After washing with PBS containing 0.05% Tween 20, the plates were incubated with HRP conjugated anti-mouse IgG-Fc (Bethyl Laboratories). The amount of IgG bound was assessed by 3,3′,5,5′-tetramethylbensidine (TMB; Biolegend) and the absorbance measured at 450 nm after 2M sulfuric acid addition.

Preparation of kidney and joint homogenate for IgG purification. Mice were bled on days 4, and 7 after anti-GBM antiserum injection. The serum was separated from the blood by serum gel tubes (BD) and incubated with Protein G high-capacity agarose beads (Thermo Fisher Scientific) for IgG purification. Paws cut above joints and kidneys were dissected, suspended in 1 mL PBS supplemented with protease inhibitor and 2 mM EDTA and cut into small pieces before being mechanically homogenized with stainless steel beads and TissueLyser II (Qiagen) for two minutes at 3 Hz/s. Homogenate was then diluted 5-fold the volume (PBS with Protein Inhibitor (Thermo) and 2 mM EDTA), filtered through 70 μm mesh, and centrifuged at 1000×g for 5 min. Supernatant was used to purify IgG with Protein G high-capacity agarose beads.

Histology. Ankle joints were dissected and incubated in the fixative and decalcifier solution Cal-Ex II for 48 hrs-72 hrs (Fisher Chemical), and embedded in paraffin. 4 μm sections were stained with hematoxylin/eosin for histological analysis. Kidneys were dissected, fixed in 10% buffered formalin and embedded in paraffin. 4 μm paraffin sections were stained with periodic acid-schiff (PAS), and hematoxylin/eosin for analysis by light microscope. 4 μm OCT (Tissue-Tek) frozen kidneys sections were fixed in acetone and stained, where indicated, with DAPI (Biolegend) or rabbit anti-mouse IgG-Fc specific-DyLight405 (Jackson ImmunoResearch) in combination with rat anti-mouse CD41-APC (Biolegend), rat anti-mouse CD62P-PE (Biolegend), and goat anti-mouse Nephrin (R&D Systems) followed by donkey anti-goat IgG-AF488 (Jackson ImmunoResearch) according to manufacturer's instructions. Slides were examined using a fluorescence microscope (Carl Zeiss).

Platelet preparation. Platelet isolation, activation and inhibition were adapted from past studies (Boilard et al., 2010, Lee et al., 2014). Healthy individuals gave informed consent and whole blood was collected in sodium citrate buffered blood collection tubes (BD) and centrifuged for 10 min at 200×g. The platelet rich plasma (PRP) in supernatant was collected and further centrifuged for 5 min at 900×g. After removing the platelet poor plasma in supernatant the pelleted platelets are resuspended in 140 mM NaCl, 3 mM KCl, 0.5 mM MgCl₂, 5 mM NaHCO₃, 10 mM D-glucose, 10 mM HEPES, pH 7.4. Platelet activation was achieved using 0.2U of Thrombin (Roche) for 5 min at 37° C. Platelet activation was inhibited using 0.25 mg of Clopidogrel for 15 min at room temperature. Platelets were pelleted by centrifugation at 1000×g for 5 min, and platelet supernatant was quantified for UDP-Galactose and CMP-SA.

Quantification of galactose and sialic acid donor in human serum. Quantitation of the glycan donor was performed on human platelet supernatant using sialyltransferase and glycosyltransferase activity kit as indicated by the manufacturer (R & D systems), with one exception. Standard curves were generated using a range of UDP-Gal and CMP-SA in order to more accurately report the concentration.

Serum Half-life Experiments. 50 μg of IVIG or B4ST6^{Fe} was intravenously administered to C57BL/6 female mice. The mice they were bled daily up to 4 days after the injection and every other day until day 10. IVIG or B4ST6^{Fe} in mice sera was detected by ELISA. Briefly, 96-well plates were coated with 5 μg/ml of anti-human IgG Fc (Bethyl Laboratories), blocked with 2% BSA in PBS, and probed with anti human IgG-HRP (20 ng/ml, Promega). 3,3,5,5-tetramethylbenzidine (TMB; Thermo Fisher Scientific) was used for the detection, and 2M sulfuric acid was used to stop the reaction.

Blood testing. 50 μg of B4ST6^{Fe} was intravenously injected to mice. After 1 week or 2 months of the administration of the enzyme the mice were bled, and whole blood and sera were sent to MGH Histopathology Research Core for complete blood count and comprehensive metabolic panel tests.

Quantification and Statistical Analysis. Data were analyzed in GraphPad Prism: *p<0.05, **p<0.01, ***p<0.005, ****p<0.001 as determined by two-way ANOVA followed by Tukey's posthoc.

TABLE 1
Materials and Resources

| | | |
|---|---|---|
| REAGENT or RESOURCE | SOURCE | IDENTIFIER |

Antibodies

| | | |
|---|---|---|
| Anti-human IgG-HRP | Promega | Cat#: W4031 |
| Anti-human B4GALT1 | Sigma-Aldrich | Cat#: HPA010807 |
| Anti-rabbit IgG-HRP | Promega | Cat#: W4011 |
| Anti-mouse IgG-Fc-HRP | Bethyl Laboratories | Cat#: A90-131A |
| Anti-human ST6GAL1 | Dr. J. Paulson | n/a |
| Anti-human IgG-Fc | Bethyl Laboratories | Cat#: A80-104A |
| Sheep IgG | BioRad | Cat#: PSPOl |
| PE anti-mouse/rat CD62P | Biolegend | Cat#: 148305 |
| APC anti-mouse CD41 Antibody | Biolegend | Cat#: 133913 |
| Mouse nephrin antibody | R & D systems | Cat#: AF3159 |
| DyLight ™ 405 AffiniPure Rabbit Anti- | Jackson | Cat#: 315-475-008 |
| Mouse IgG, Fcγ fragment specific | ImmunoResearch | |
| Alexa Fluor ® 488 AffiniPure Donkey | Jackson | Cat#: 705-545-003 |
| Anti-Goat IgG (H + L) | ImmunoResearch | |

Chemicals, Peptides, and Recombinant Proteins

| | | |
|---|---|---|
| pcDNA ™3.4 TOPO ™ TA Cloning Kit | Thermo Fisher | Cat#: A14697 |
| Expi293 ™ Expression System Kit | Thermo Fisher | Cat#: A14635 |
| Pierce ™ Protein G Plus Agarose | Thermo Fisher | Cat#: 22852B |
| Sialidase A | ProZyme | Cat#: GK80040 |
| β1-4 Galactosidase S | New England Biolab | Cat#: P0745L |
| UDP-α-D-Galactose, Disodium Salt | Millipre Sigma | Cat#: 670111 |
| | | CAS#: 137868-52-1 |
| CMP-Sialic acid (Cytidine-5′- | Nacalai USA, Inc. | Cat#: 10432-24 |
| monophospho-N-acetylneuraminic Acid | | |
| Disodium Salt) | | |
| Pierce ™ Protein-Free (TBS) Blocking | Thermo Fisher | Cat#: 37570 |
| Buffer | | |
| Biotinylated Sambucus Nigra Lectin (SNA) | Vector Laboratories | Cat#: B-1305 |
| Biotinylated Erythrina Cristagalli Lectin | Vector Laboratories | Cat#: B-1145 |
| (ECL) | | |
| PNGase F | New England Biolab | Cat#: P0704L |
| Endo S | New England Biolab | Cat#: P0741L |
| GlykoClean ™ G Cartridges | ProZyme | Cat#: GC250 |
| GlykoClean ™ S-plus Cartridges | ProZyme | Cat#: GC210 |
| Anthranilamide (2-AB) | Sigma-Aldrich | Cat#: A89804 |
| NTS (Sheep Anti-Rat Glomeruli (Anti- | Probetex | Cat#: PTX001-S |
| GBM) Serum) | | |
| TMB Substrate | Biolegend | Cat#: 421101 |
| Protease Inhibitor Mini Tablets | Thermo Fisher | Cat#: 88665 |
| Cal-Ex ™ II Fixative/Decalcifier | Fisher Chemical | Cat#: CS511-1D |
| QuikChange II XL Site-Directed | Agilent | Cat#: 200521 |
| Mutagenesis Kit | | |
| Clopidogrel | Selleck Chemicals | Cat#: S1415 |
| DAPI (4′,6-Diamidino-2-Phenylindole, | Biolegend | Cat#: 422801 |
| Dilactate) | | |

Critical Commercial Assays

| | | |
|---|---|---|
| Urea Nitrogen (BUN) Liqui-UV Test | Stanbio Laboratory | Cat#: 2020-430 |
| Sialyltransferase Activity Kit | R & D systems | Cat#: EA002 |
| Glycosyltransferase Activity Kit | R & D systems | Cat#: EA001 |

Experimental Models: Cell Lines

| | | |
|---|---|---|
| Expi293F ™ Cells | Thermo Fisher | RRID:CVCL_D615 |
| | | Cat#: A14527 |

Experimental Models: Organisms/Strains

| | | |
|---|---|---|
| Mouse: C57BL/6J | The Jackson | RRID: |
| | Laboratory | IMSR_JAX:000664 |
| Agilent AdvanceBio Glycan Mapping | Agilent | Cat#: 683775-913 |
| column (120 Å, 2.1 × 150 mm, 2.7 μm) | | |

TABLE 2
Oligonucleotides
Primers: target gene/primer name/sequence (5′→3′)
Human ST6GAL1/hST6:EFQ_Fwd
AAA(AAGCTT)ATGGAATTCCAGGTGTTAAAGAGTCTGGGG
Human ST6GAL1/hST6GAL1(K24)
GGCGCGCCAATGAAGGAAAAGAAGAAAGGGAGTTACTATGATTCC
Human ST6GAL1/hST6GAL1(K27)
AAGCTTATGAAGAAAGGGAGTTACTATGATTCCTTTAAATTGC
Human ST6GAL1/hST6GAL1(Y32)
AAGCTTATGTATGATTCCTTTAAATTGCAAACCAAGGAATTCC
Human ST6GAL1/hST6_Rev
AAA(CTCGAG)TTAGCAGTGAATGGTCCGGAAGCC
Human B4GALT1/hB4:ECD_Fwd
AAA(AAGCTT)ATGGGCCGCGACCTGAGCCGCC
Human B4GALT1/hB4_Rev
AAA(CTCGAG)CTAGCTCGGTGTCCCGATGTCC
Human ST6GAL1/hST6_SDM_C353A_F
CAAGCGCAAGACTGACGTGGCCTACTACTACCAGAAGTTC
Human ST6GAL1/hST6_SDM_C353A_R
GAACTTCTGGTAGTAGTAGGCCACGTCAGTCTTGCGCTTG
Human ST6GAL1/hST6_SDM_C364A_F
GTTCTTCGATAGTGCCGCCACGATGGGTGCCTAC
Human ST6GAL1/hST6_SDM_C364A_R
GTAGGCACCCATCGTGGCGGCACTATCGAAGAAC
IL2 signal sequence/Kozac_Il2ss_Fwd
AAACTCGAGGCCACCATGTACAGGATGCAACTCCTGTC
Restriction sites for HindIII (AAGCTT) and XhoI (CTCGAG) are indicated by brackets.

### Example 2: Engineering Soluble Glycosyltransferases

ST6GAL1 catalyzes attachment of α2,6 sialic acid to galactose on N-linked glycans (Meng et al., 2013). Sialylation by ST6GAL1 typically occurs in the trans-Golgi where the glycosyltransferase is anchored by a transmembrane domain (TMD, FIG. 1C). A β-Secretase (BACE1) cleavage site is present in the luminal domain of ST6GAL1 at EFQ41-43, which results in ST6GAL1 secretion (FIGS. 1C and 8) (Woodard-Grice et al., 2008). Remarkably, recent studies have suggested a soluble ST6GAL1 contributes to sialylation of IgG Fc glycans (Jones et al., 2012, Sugimoto et al., 2007, Jones et al., 2016). The extraordinary effects of sialylation on IgG biology prompted us to explore the therapeutic potential of glycoengineering IgG in vivo.

To this end, glycosylation enzymes were fused to human IgG1 Fc, a common approach to generate soluble forms of membrane proteins. Expression constructs with fusions of Fc and ST6GAL1 that included the region upstream of EFQ41-43 in the amino acid sequence resulted in multiple protein products, consistent with BACE1 activity (FIG. 8). However, when the Fc was linked directly to E41, omitting the first 40 amino acids of ST6GAL1, a single product was generated (ST6^{Fc}, FIGS. 1C and 8B). Because sialylation efficiency improves with increased galactose content (Anthony et al., 2008a), a similar approach was used for the B4GALT1 enzyme responsible for attachment of galactose to the IgG Fc glycan. The engineered fusions of B4GALT1 luminal domains with human IgG1 Fc resulted in a single protein product (B4^{Fe}, FIGS. 1C and 8B). The engineered glycosyltransferases were determined to be of the correct molecular weight, and were recognized by antibodies specific for B4GALT1, ST6GAL1, and human IgG by immunoblotting (FIG. 8D).

The activity of engineered enzymes in vitro using fetuin, a highly glycosylated protein, as a target for glycoengineering was examined. Fetuin was first treated with glycosidases that remove sialic acid and galactose residues, generating asialylated, galactosylated (G2) and agalactosylated (G0) fetuin (FIG. 1D). The G0 and G2 fetuin was incubated with B4^{Fe}, ST6^{Fe}, or with both enzymes (B4ST6^{Fc}) and sugar-nucleotide donors (UDP-Galactose (UDP-Gal) and CMP-Sialic Acid (CMP-SA) for galactose and sialic acid, respectively). Linkage-specific glycosylation was examined by lectin blotting, and revealed that B4^{Fe} efficiently attached terminal galactose in β1,4 linkages, and ST6^{Fe} attached α2,6 terminal sialic acid (FIG. 1D). B4ST6^{Fc} attached β1,4 galactose when incubated with a galactose donor (UDP-Gal, FIG. 1D). As mentioned above, studies demonstrated galactosylation increases efficiency of sialylation, presumably by increasing the number of potential sialylation sites (Anthony et al., 2008a), and B4ST6^{Fe} efficiently sialylated fetuin when incubated with both galactose and sialic acid donors (FIG. 1D). Further, B4ST6^{Fe} transferred galactose and sialic acid to both mouse and human IgG (FIG. 1E).

### Example 3: Anti-inflammatory Activity of In Vivo Sialylation

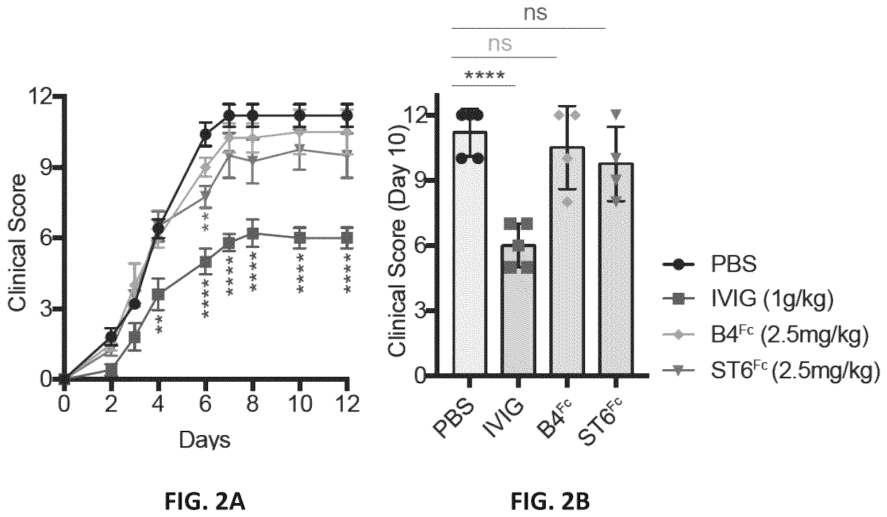

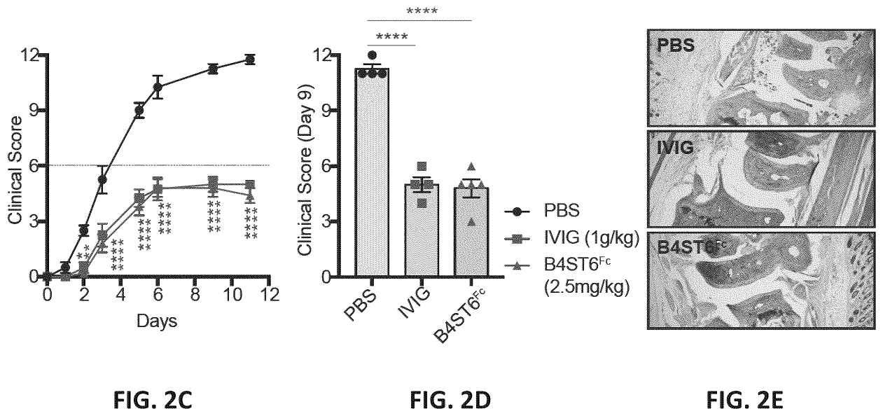

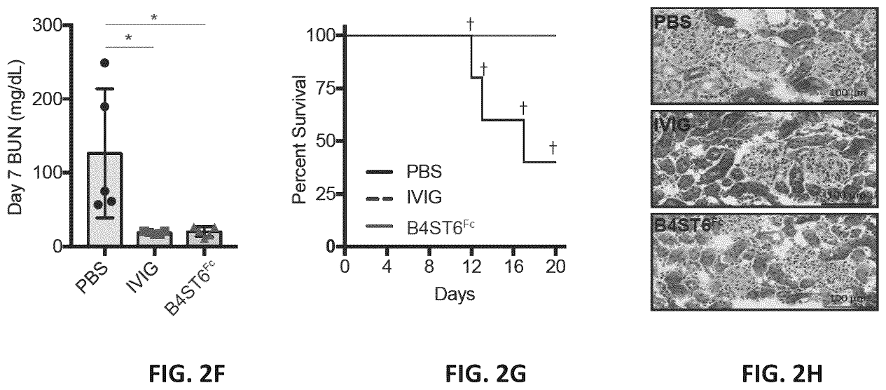

The ability of these engineered glycosylation enzymes to attenuate inflammation in vivo was tested. Mice were given arthritogenic K/B×N sera, which initiates joint inflammation mediated primarily by IgG1-autoantibodies that presents with edema and inflammatory cell infiltration within days after treatment (Korganow et al., 1999). Animals also received PBS, high dose IVIG (1 g/kg), B4^{Fe} (2.5 mg/kg), ST6^{Fe} (2.5 mg/kg), or both B4^{Fe} and ST6^{Fc} (B4ST6^{Fe}, 2.5 mg/kg, FIGS. 2A-2D). The arthritogenic sera induced robust inflammation in PBS-treated animals as measured by clinical score, while inflammation was attenuated by IVIG (FIGS. 2A-2B). Neither B4^{Fe} nor ST6^{Fe} individually was able to reduce induced inflammation. However, when the engineered enzymes were co-administered (B4ST6^{Fe}, 2.5 mg/kg), inflammation was significantly attenuated to a similar level achieved by IVIG (FIGS. 2C-2D). The inflammatory infiltrate to the joint, and tissue destruction 7 days after treatment was markedly reduced in IVIG and B4ST6^{Fc}-treated animals compared to PBS-treated controls (FIG. 2E). Together, these results demonstrate that administration of enzymes that attach both galactose and sialic acid are effective at attenuating passive autoimmune arthritic inflammation in vivo.

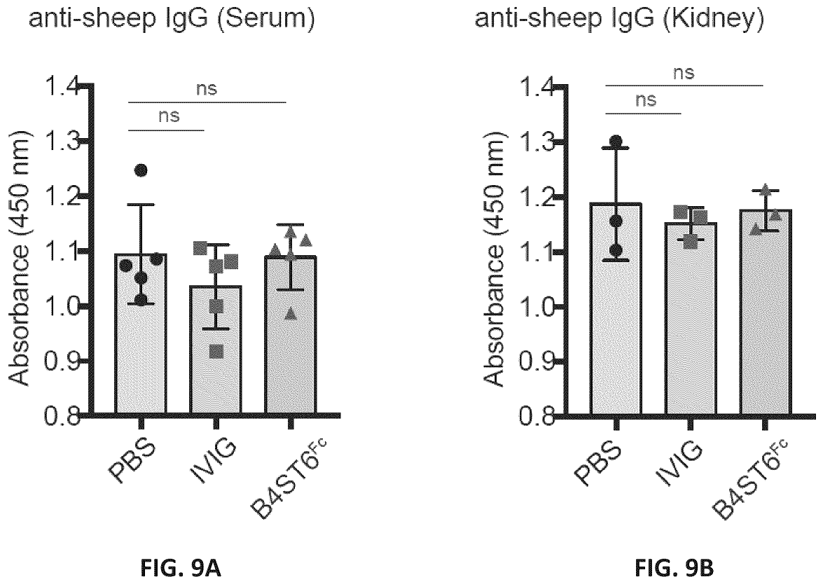

To extend these findings to an active model of autoimmune disease, a model of Goodpasture's disease that results in nephrotoxic nephritis (NTN), driven predominantly by IgG2b-based immune complexes deposited in the kidneys resulting in kidney damage (Lemer et al., 1967, Schrijver et al., 1990, Kaneko et al., 2006a) was used. Administration of B4ST6^{Fe} suppressed kidney pathology as effectively as IVIG, as measured by blood urea nitrogen (BUN) levels at day 7 (FIG. 2F), and extended survival (FIG. 2G). Indeed, inflammatory cell infiltration into the kidneys at day 7 was reduced by B4ST6^{Fe} and IVIG, compared to PBS controls (FIG. 2H). Neither IVIG nor B4ST6^{Fe} affected the induced pathogenic antibody response, as measured by serum or kidney IgG titers (FIGS. 9A-9B). Together, these results demonstrate that in vivo sialylation by B4ST6^{Fe} effectively ameliorates autoantibody-mediated kidney destruction at a dose that is 400-fold lower than immunomodulatory high dose IVIG.

### Example 4: Requirements for In Vivo Sialylation

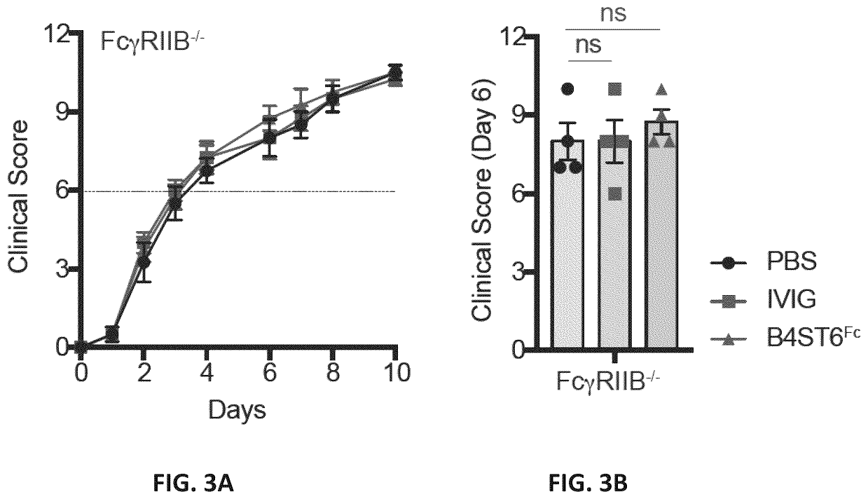

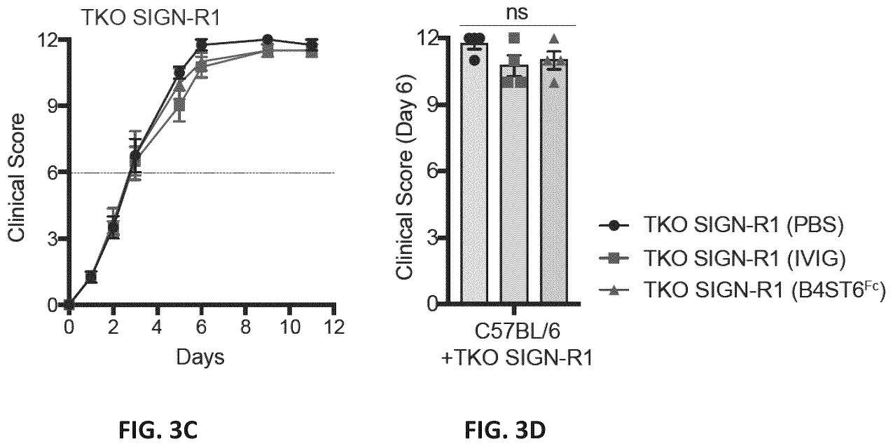

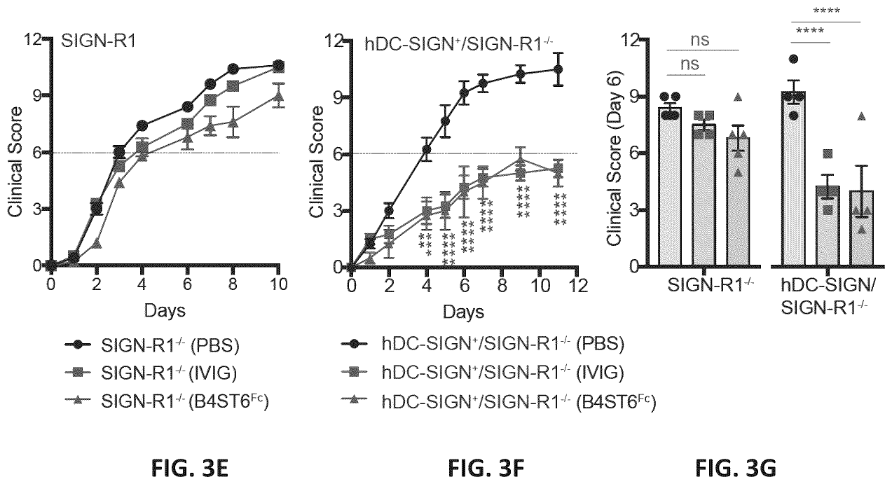

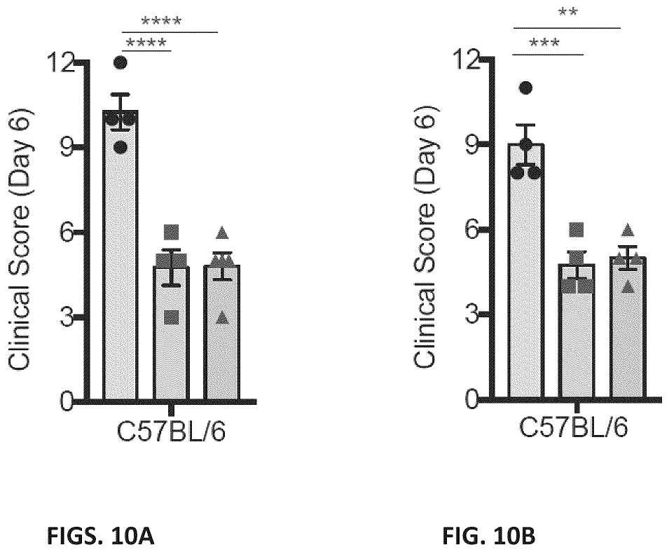

The requirements of the inhibitory FcγRIIB for the anti-inflammatory activity of IVIG and sialylated IgG Fc have been supported by functional studies using murine models (Samuelsson et al., 2001, Bruhns et al., 2003, Anthony et al., 2011, Schwab et al., 2014), and increased surface expression of FcγRIIB on leukocytes as been observed following administration of high dose IVIG to chronic inflammatory demyelinating polyneuropathy patients (Tackenberg et al., 2009). Further, sialylated IgG Fc were shown to require murine SIGN-R1 or human DC-SIGN to suppress inflammation (Anthony et al., 2011, Anthony et al., 2008b, Schwab et al., 2012). Thus, sialylation of IgG converts receptor preference to type II FcγRs, and ligation of these receptors by sialylated IgG culminates in the upregulation of the inhibitory FcγRIIB on inflammatory cells (Pincetic et al., 2014). Experiments were also performed to determine whether in vivo sialylation suppressed inflammation through a pathway similar to IVIG. K/B×N sera along with PBS, IVIG, or B4ST6^{Fe} was administered to wild type or FcγRIIB^{-/-} mice, and paw inflammation over the next several days was tracked. Neither IVIG nor B4ST6^{Fe} suppressed inflammation relative to PBS (FIGS. 3A, 3B and 10A) in FcγRIIB^{-/-} mice demonstrating a requirement for this receptor for their anti-inflammatory activity. Next, an antibody was administered to mice that results in transient knockdown of SIGN-R1 (TKO SIGN-R1 (Kang et al., 2004)), which has been shown to attenuate IVIG and sialylated IgG anti-inflammatory activity in vivo (Anthony et al., 2008b). This resulted in no observed differences in mice treated with K/B×N and PBS, IVIG, or B4ST6^{Fe}, indicating SIGN-R1 perturbation inhibited the anti-inflammatory activity of both IVIG and in vivo sialylation (FIGS. 3C, 3D, and 10B).

Next, K/B×N sera was administered to SIGN-R1^{-/-} and human DC-SIGN transgenic mice that were crossed to a SIGN-R1^{-/-} background (hDC-SIGN⁺/SIGN-R1^{-/-}, FIGS. 3E, 3F, 3G). The mice also received PBS, IVIG, or B4ST6^{Fe}, and inflammation was monitored over the next several days. Transfer of K/B×N sera along with PBS treatment resulted in robust inflammation in both genotypes (FIG. 3E, 3F). SIGN-R1^{-/-} animals were not protected from induced arthritis by IVIG or B4ST6^{Fc} (FIG. 3E). However, both IVIG and B4ST6^{Fe} attenuated induced-inflammation in hDC-SIGN⁺/SIGN-R1^{-/-} mice (FIG. 3F). Together, these results suggest that IVIG and in vivo sialylation by engineered galactosyl- and sialyltransferases suppress inflammation through similar pathways.

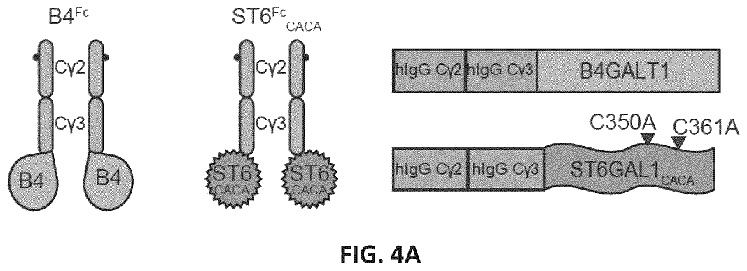

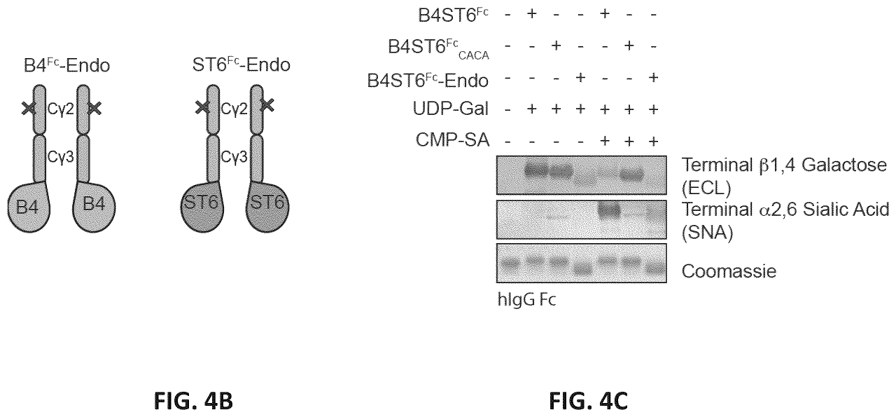

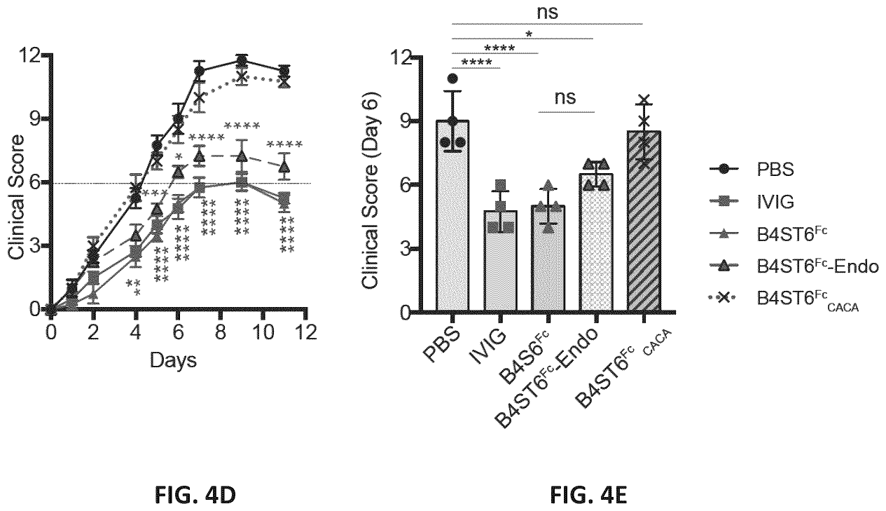

The shared receptors and pathways between IVIG and in vivo sialylation raised the possibility that the Fc glycan on the engineered enzymes, and not enzymatic activity, were responsible for the in vivo anti-inflammatory activity. Therefore, an enzymatically inactive ST6^{Fe} by mutating two enzymatic-domain cysteine residues to alanine was generated (C350A, C361A, ST6^{Fc}_{CACA}, FIG. 4A (Meng et al., 2013)). In parallel, B4^{Fe} and ST6^{Fe} were treated with the IgG Fc-specific endoglycosidase, EndoS to remove the Fc glycan (B4ST6^{Fc}-Endo, FIG. 4B) (Collin and Olsen, 2001). Enzymatic removal of the Fc glycan has been shown to ablate interactions of IgG and FcγRs (Allhorn et al., 2010, Benkhoucha et al., 2012, Yang et al., 2010). Importantly, B4ST6^{Fc}_{CACA} was unable to transfer sialic acid to human IgG Fc in vitro, although galactosyltransferase activity remained intact (FIG. 4C). However, B4ST6^{Fc}-Endo retained galactosyl- and sialyltransferase activity (FIG. 4C). These enzymes were tested for anti-inflammatory activity in vivo. K/B×N sera was administered to mice, which also received PBS, IVIG, B4ST6^{Fc}, B4ST6^{Fc}_{CACA}, or B4ST6^{Fc}-Endo. K/B×N transfer induced robust inflammation, which was attenuated by IVIG, B4ST6^{Fc}, and B4ST6^{Fc}-Endo (FIGS. 4D, 4E). However, B4ST6^{Fc}_{CACA} was unable to suppress induced inflammation (FIGS. 4D, 4E). These results demonstrate that transferase activity, and not Fc glycans on engineered enzymes are required for suppression of inflammation in vivo.

### Example 5: Site-Specific Sialylation In Vivo

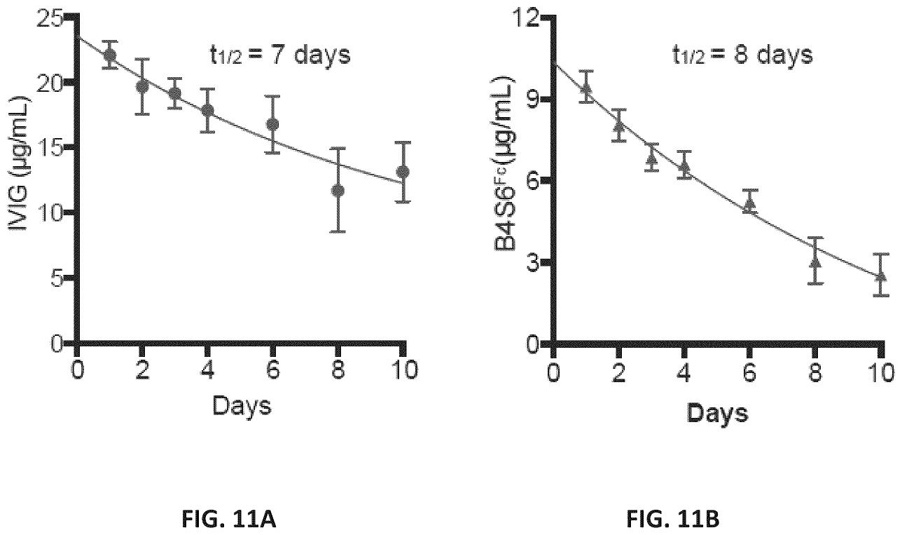

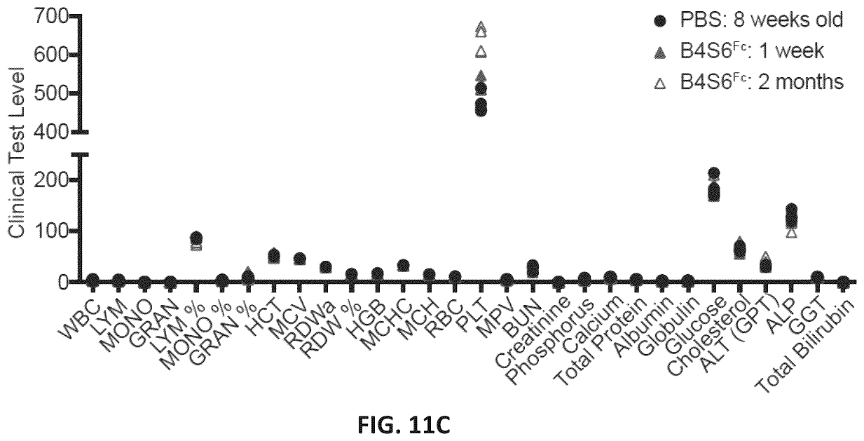

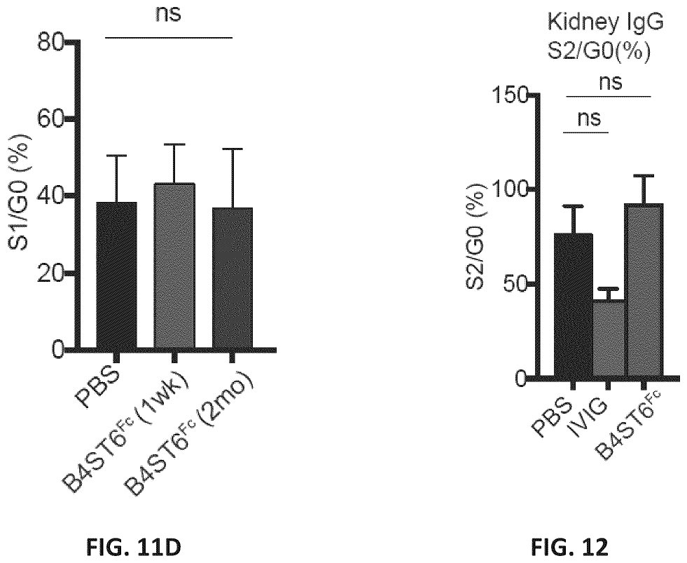

While administration of the engineered glycosyltransferases potently suppressed autoimmune inflammation, a potential undesirable side effect may be off-target glycan modification. In general, low levels of sialic acid are found on IgG Fc glycans, as 5-10% of total IgG in healthy individuals have sialylated Fc glycans. Most complex antennary glycans on cellular and soluble glycoproteins are highly sialylated limiting potential off-target effects of in vivo sialylation (Kaneko et al., 2006b, Youings et al., 1996). Nonetheless, experiments were performed to examine toxicity and systemic glycosylation following B4ST6^{Fe} administration in vivo. The half-life of B4ST6^{Fe} in circulation was similar to IVIG (8 and 7 days, respectively, FIGS. 11A, 11B), suggesting that the Fc portion of these molecules similarly controls serum half-life in vivo. Experiments were also performed to examine the homeostatic impact of B4ST6^{Fe} one week and two months after administration (FIG. 11C). No detrimental effect was noted on complete blood count (CBC) analysis of red blood cells (RBC), white blood cells (WBC) and platelets. Serum glucose and calcium levels remained within normal range, while kidney and liver function was unaltered on comprehensive metabolic panel (CMP) analysis. The analyses revealed little differences between PBS-treatment and either B4ST6^{Fc}-treatment group, and suggested that administration of B4ST6^{Fe} is not toxic (FIG. 11C). Further experiments were performed to examine the glycosylation of IgG and total serum proteins one week and two months following administration of B4ST6^{Fc} (FIG. 11D). Minimal changes in glycosylation were observed in B4ST6^{Fe} treated animals compared to PBS-treated controls, suggesting that minimal off-target effects result from in vivo sialylation.

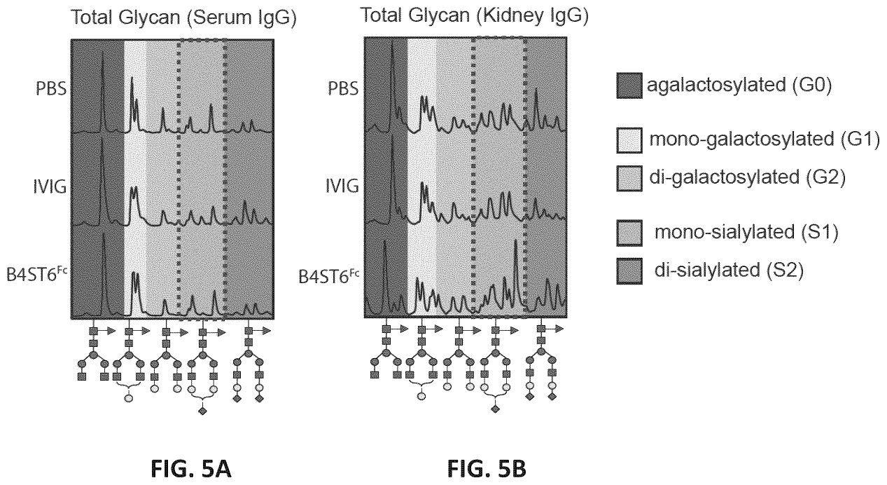

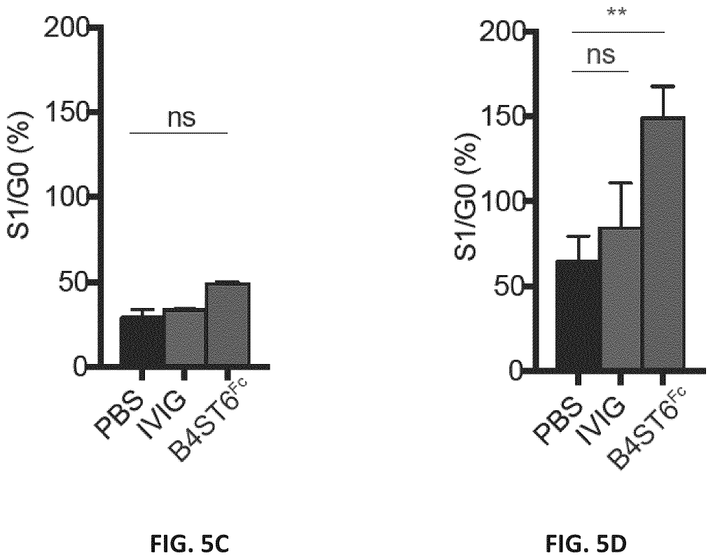

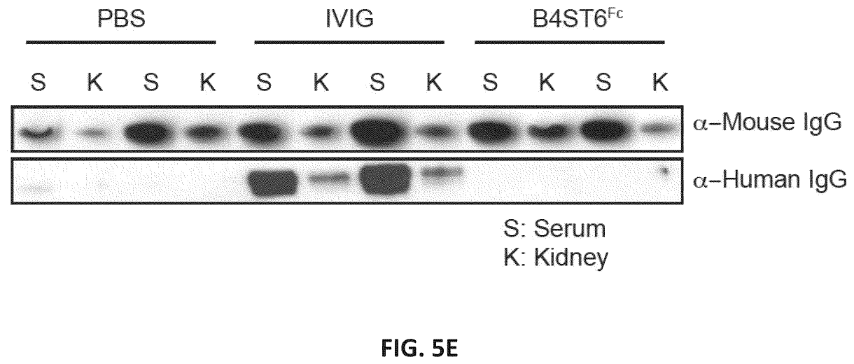

Because administration of both engineered enzymes was anti-inflammatory in vivo, but did not notably alter serum IgG or protein glycosylation during homeostatic conditions, experiments were performed to examine glycosylation during an inflammatory response. NTN was induced in a panel of mice that received PBS, IVIG, or B4ST6^{Fe}, and the total N-linked glycosylation was examined. Seven days after disease induction, no differences in circulating IgG in PBS, IVIG, or B4ST6^{Fc} treated mice were observed, consistent with the findings where B4ST6^{Fe} does not affect glycosylation of circulating glycoproteins (FIG. 5A). Intriguingly, it has been observed that there was an increase in sialylation of IgG recovered from the kidneys of B4ST6^{Fc}-treated animals, compared to IgG recovered from kidneys of PBS or IVIG treated mice (FIG. 5B). The ratio of anti-inflammatory monosialylated to inflammatory agalactosylated IgG Fc glycans (S1/G0%) of serum revealed no differences in sialic acid content on serum IgG, while IgG recovered from the kidney had significant increases in sialylation, but no change in disialylated to agalactosylated kidney IgG Fc glycans (FIGS. 5C, 5D, 12). Immunoblot analysis of IgG purified from the serum (S) and kidney (K) of PBS, IVIG, or B4ST6^{Fe} treated animals revealed measurable levels of mouse IgG in all samples, while human IgG Fc were only detectable in mice treated with IVIG (FIG. 5E). This indicates that the analysis of IgG glycans on PBS and B4ST6^{Fe} treatment group was restricted to endogenous mouse IgG Fc, and not the Fc of the engineered glycosyltransferases. Together, these results demonstrate that endogenous IgG at the site of inflammation is sialylated selectively by B4ST6^{Fc}.

### Example 6: In Vivo Sialylation Requires Platelet Activation

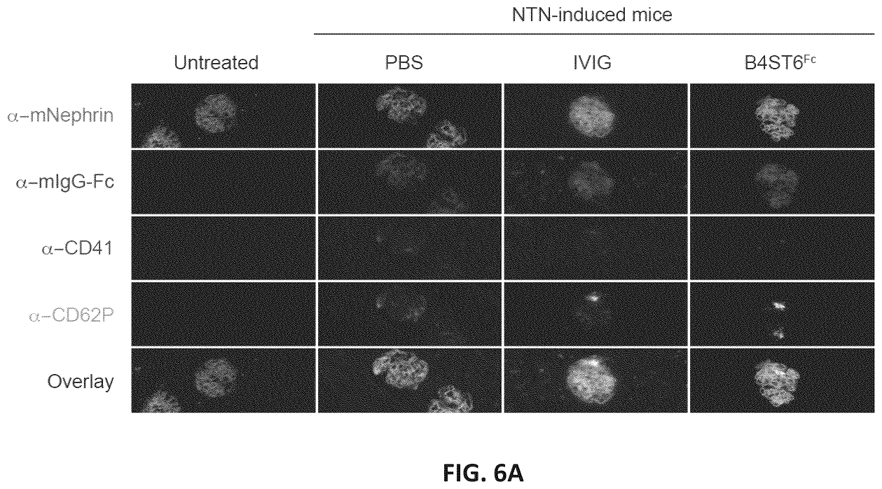

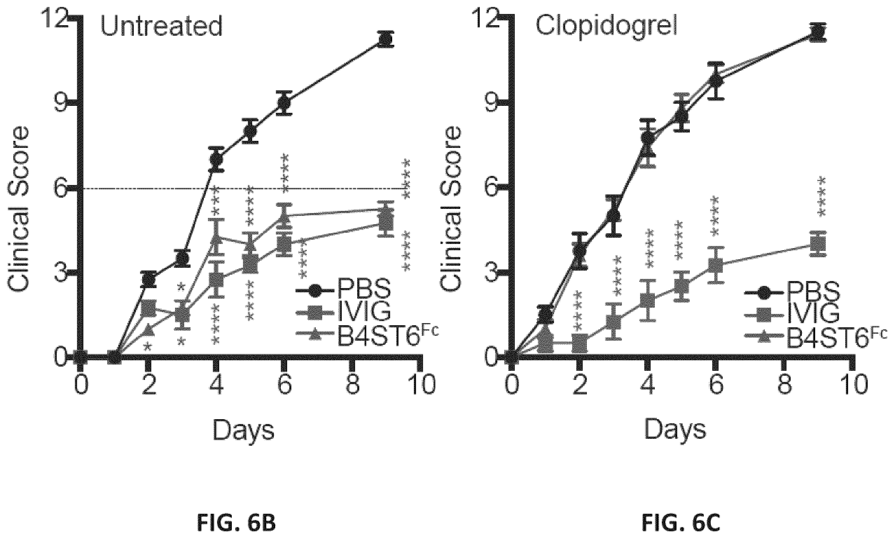

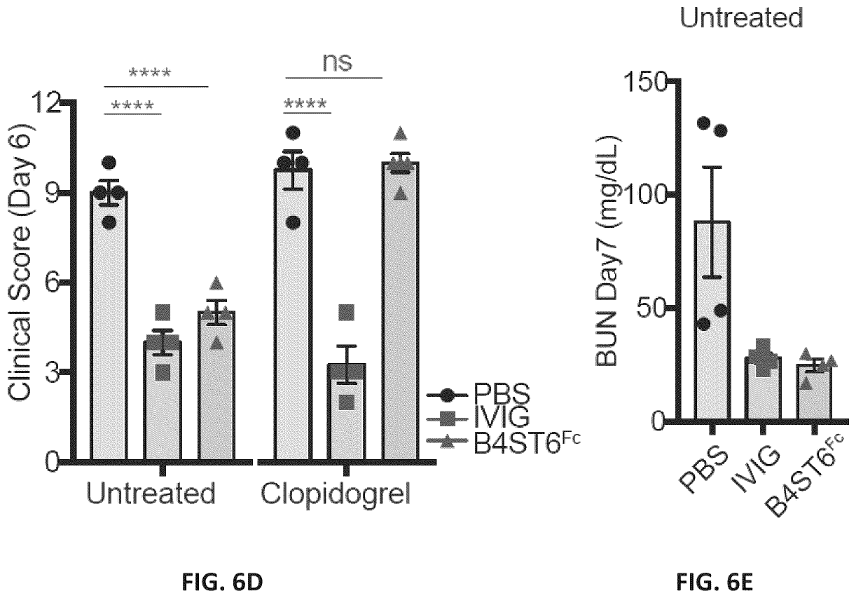

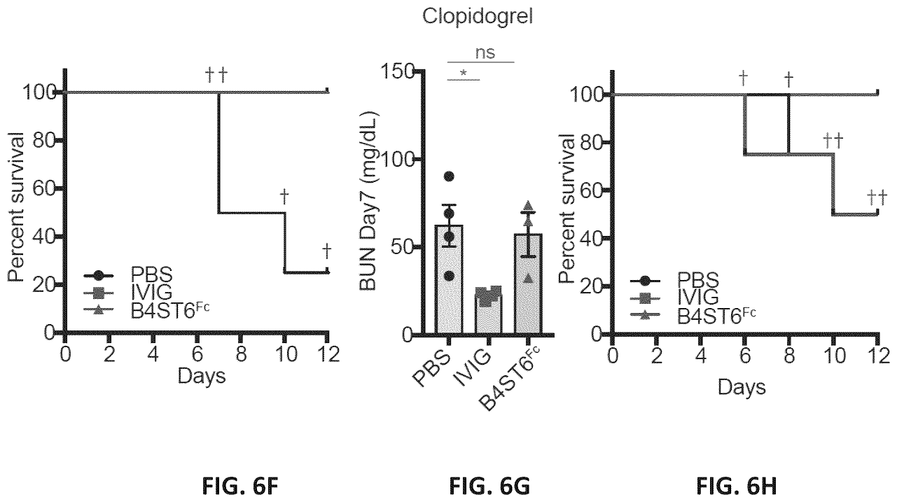

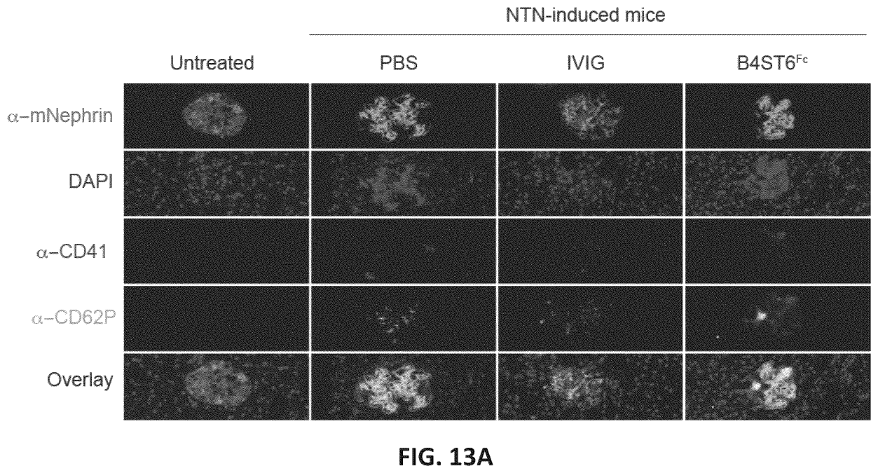

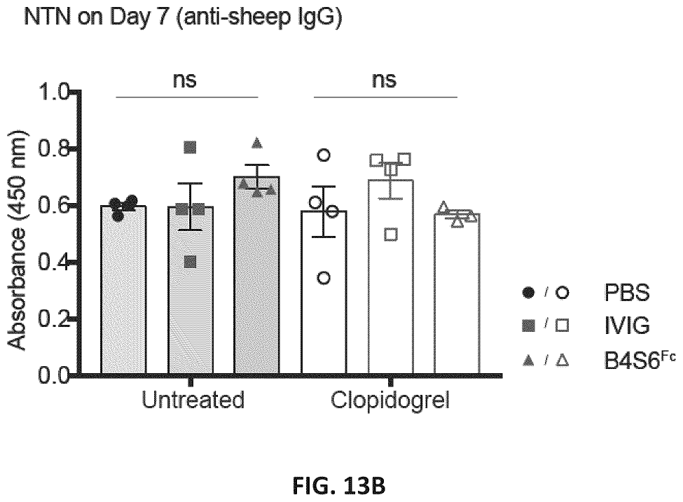

Studies that have examined the activity of soluble ST6GAL1 implicated platelets as donors of CMP-sialic acid (CMP-SA), which is required for sialylation reactions (Jones et al., 2016, Jones et al., 2012, Lee et al., 2014). Therefore, experiments were performed to determine whether platelets provided sugar-nucleotide donors for B4ST6^{Fc}. Indeed, CD41+ platelets were detected in the glomeruli of mouse kidneys in which NTN had been induced, but not in the glomeruli of untreated kidneys, consistent with previous studies demonstrating platelet recruitment to sites of inflammation (FIGS. 6A and 13A) (Devi et al., 2010, Boilard et al., 2010). Further, CD41⁺ platelets in NTN-inflamed kidneys also expressed the platelet activation marker, CD62P (FIGS. 6A, 13A). Treatment with PBS, IVIG, or B4ST6Fc during NTN-inflammation did not affect accumulation and activation of platelets (FIGS. 6A, 13A).

Experiments were also performed to determine whether platelet activation was required for the anti-inflammatory activity of in vivo sialylation. Mice were given clopidogrel (10 mg/kg) daily two days prior to administration of K/B×N sera to prevent platelet activation (Pucci et al., 2016). The mice also received PBS, IVIG, or B4ST6^{Fe} and inflammation was monitored over the next several days. Clopidogrel treatment did not affect induced-inflammation of PBS-treated animals (FIGS. 6B-6D). IVIG suppressed inflammation in the presence of clopidogrel (FIGS. 6B-6D). However, B4ST6^{Fe} was unable to attenuate inflammation when given coordinately with Clopidogrel (FIGS. 6C, 6D).

In an effort to extend these results, NTN was induced in mice that were administered clopidogrel (FIGS. 6E-6H) that then received PBS, IVIG, or B4ST6^{Fc} Importantly, these treatments did not affect the anti-sheep IgG titers in the treated mice (FIG. 13B). Induction of NTN caused kidney damage in clopidogrel and PBS-treated animals, as measured by blood urea nitrogen levels and survival (FIGS. 6G, 6H). IVIG protected treated mice from kidney disease, regardless of clopidogrel treatment (FIGS. 6E-H). However, B4ST6^{Fe} was ineffective at attenuating disease when given along with clopidogrel (FIGS. 6G, 6H). Together, these results demonstrate that the anti-inflammatory activity of B4ST6^{Fe}, but not IVIG, is dependent on platelet activation in vivo.

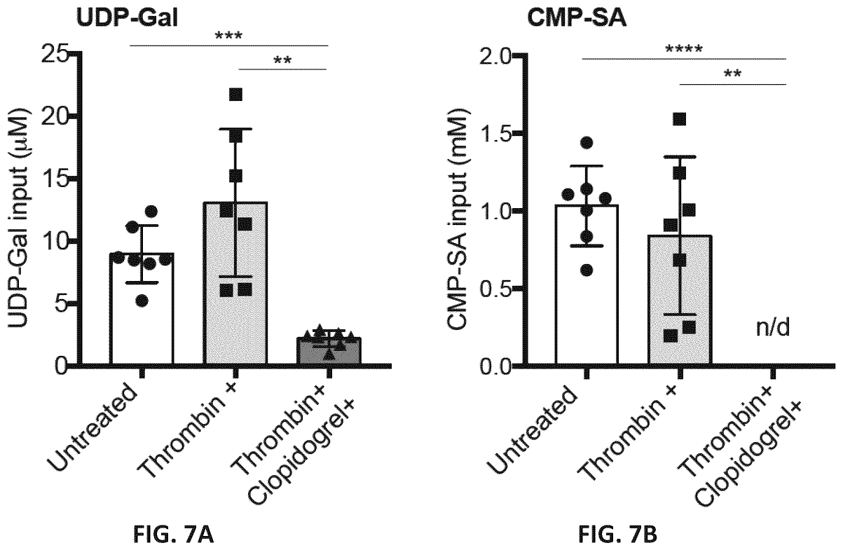

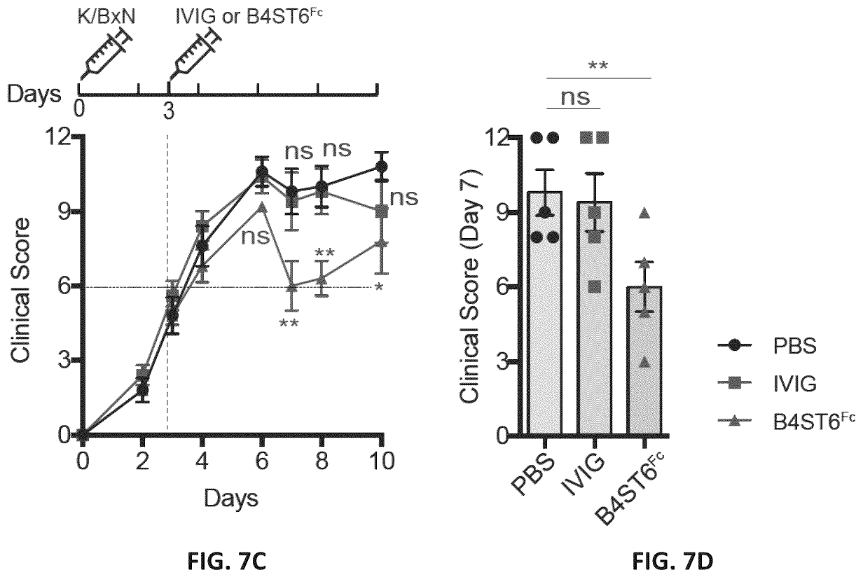

Further experiments were performed to determine whether human platelets released sugar-nucleotide donors required for sialylation, and generated platelet-enriched plasma (PRP) from healthy donors (Lee et al., 2014, Jones et al., 2016, Tan et al., 2016). Platelets was left untreated, activated (Thrombin+), or activated following clopridogrel treatment (Clopridogrel+/Thrombin+), and assayed for release of sialic acid- and galactose-nucleotide donors (CMP-SA, UDP-Gal). Indeed, human platelets released both sialic acid- and galactose-nucleotide donors upon activation, and the release was significantly inhibited by clopidogrel (FIGS. 7A, 7B). Intriguingly, activation increased galactose-nucleotide donor release, but not sialic acid-donor release.

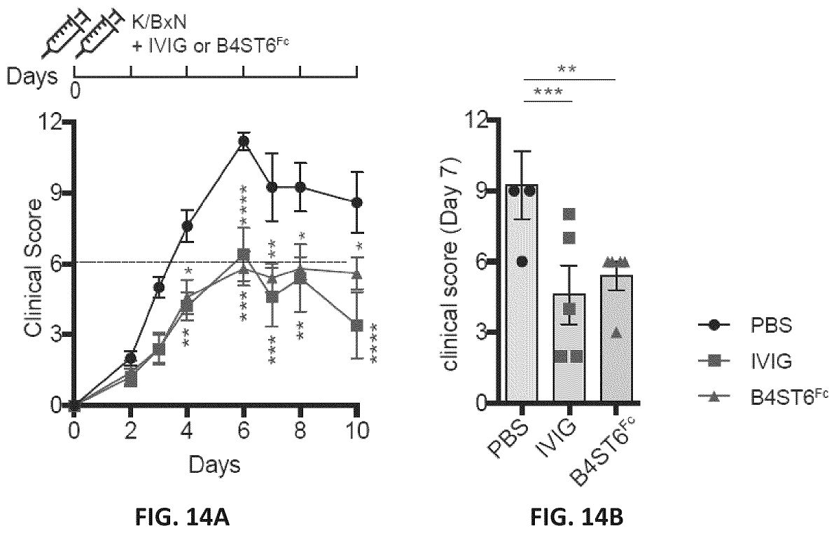

A successful anti-inflammatory therapeutic is required to suppress ongoing inflammation. To determine whether in vivo sialylation was effective therapeutically, mice were treated with arthritis-inducing sera and then given PBS, IVIG, or B4ST6^{Fc} on day 0 or day 3 after induction of arthritis. IVIG and B4ST6^{Fc}, but not PBS, were effective at reducing induced arthritis when administered on day 0 (FIGS. 14A, 14B). However, IVIG was unable to suppress induced arthritis when administered day 3 after disease induction (FIGS. 7C, 7D). Importantly, mice treated with arthritogenic sera and B4ST6^{Fe} on day 3 exhibited significantly reduced inflammation on day 7 and 8 compared to IVIG- and PBS-treated groups (FIGS. 7C, 7D). These results reveal that B4ST6^{Fe} is able to effectively attenuate autoantibody-induced inflammation in a therapeutic fashion, which was unachievable with IVIG in this model, consistent with previous results (Bruhns et al., 2003).

### Example 7: Heterodimerization of B4^{Fe} and ST6^{Fe}

In order to achieve heterodimerization of B4^{Fe} and ST6^{Fe}, “knobs-into-holes” (KIH) mutations were introduced in each heavy chain, CH3 domains. Specifically, Y349C/T366S/L368A/Y407V and S354C/T366W point mutations were introduced in B4^{Fc} (SEQ ID NO: 42) and ST6^{Fc} (SEQ ID NO: 43), respectively.

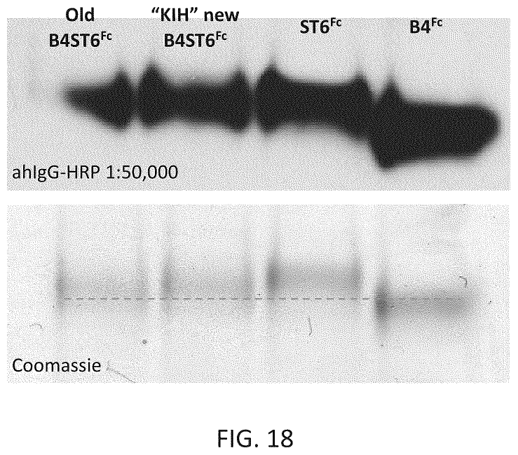

FIG. 18 shows western blot results (with anti-human IgG) and Coomassie Gel stain of the original B4ST6^{Fe} and new “knobs-into-holes” B4ST6^{Fc}. In the figure, ST6^{Fe} stands for ST6^{Fe} homodimers. B4^{Fe} stands for B4^{Fe} homodimers. The original B4ST6^{Fe} does not have the knobs-into-holes mutations, thus it is likely it is a mixture of ST6^{Fe} homodimers, B4^{Fe} homodimers, and B4^{FC}ST6^{Fe} heterodimers. The “KIH” B4ST6^{Fc} are B4^{FC}ST6^{Fe} heterodimers and ran slightly higher in a gel as shown in Coomassie gel.

### Example 8: Testing B4^{Fc}ST6^{Fc} Heterodimers in Arthritis Model

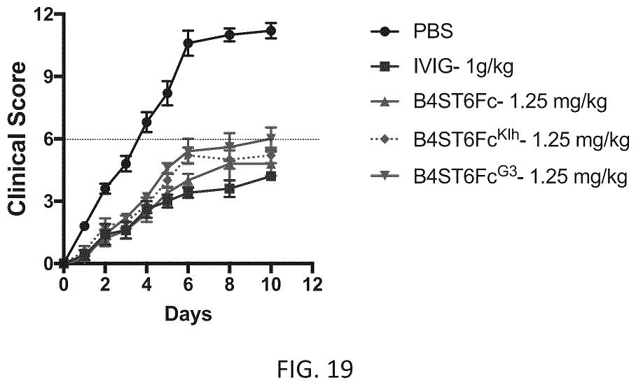

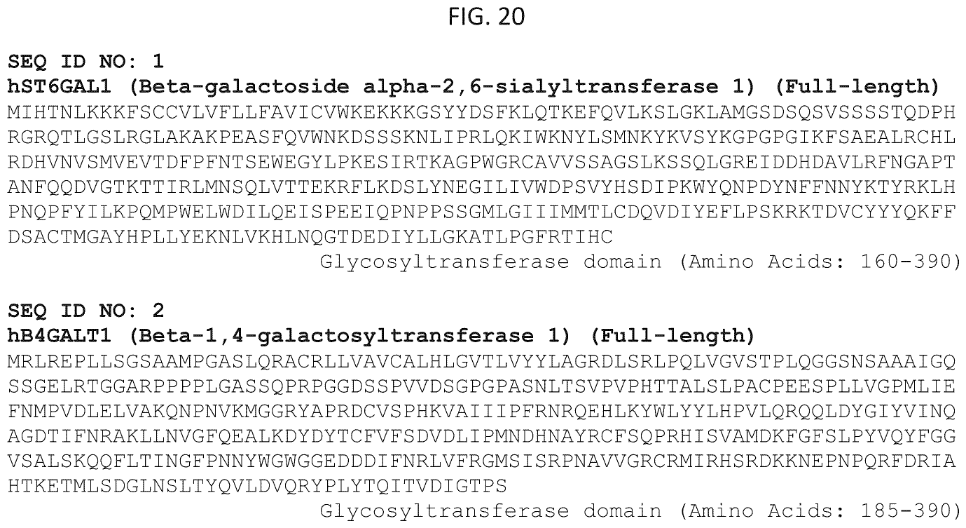

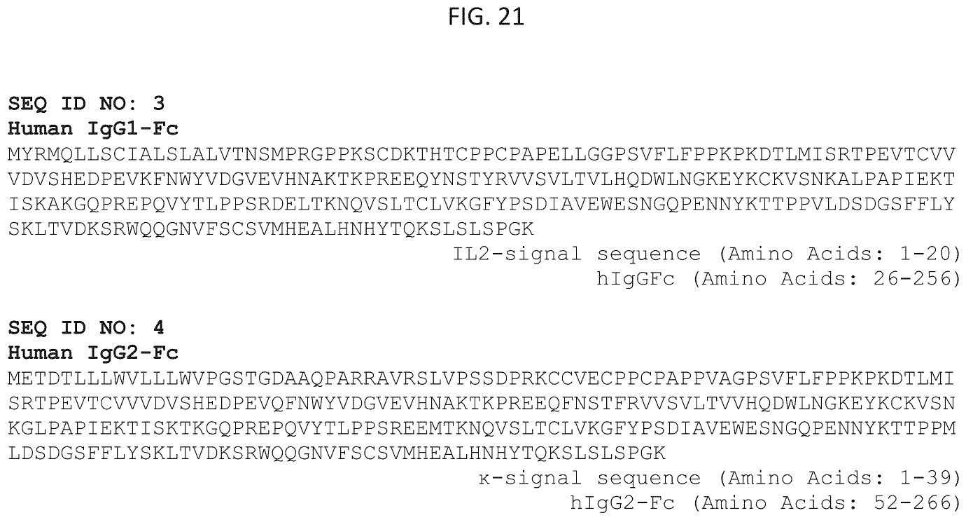

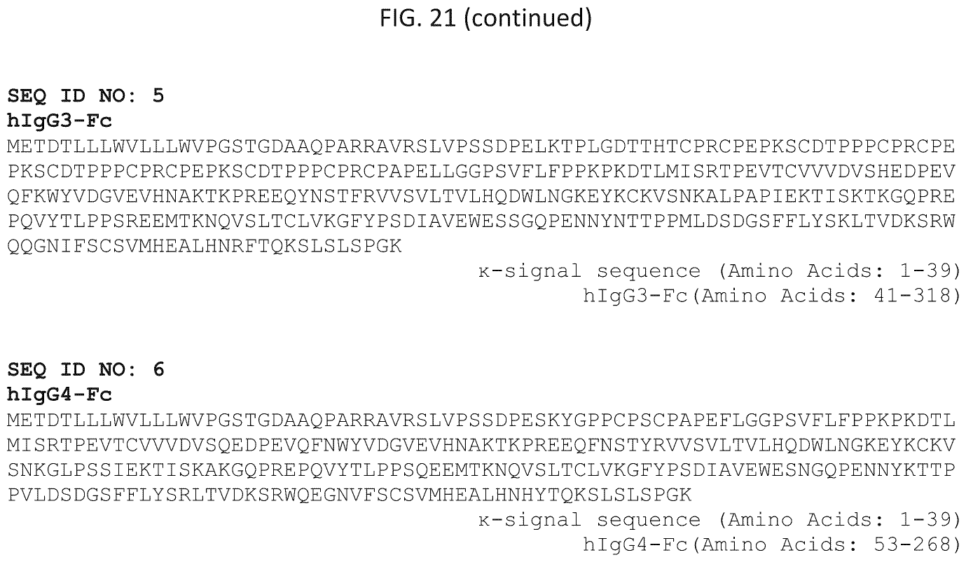

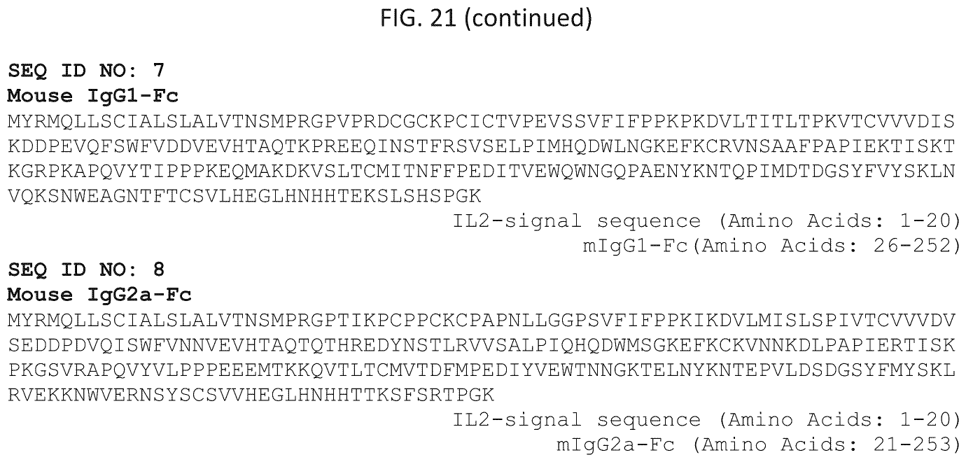

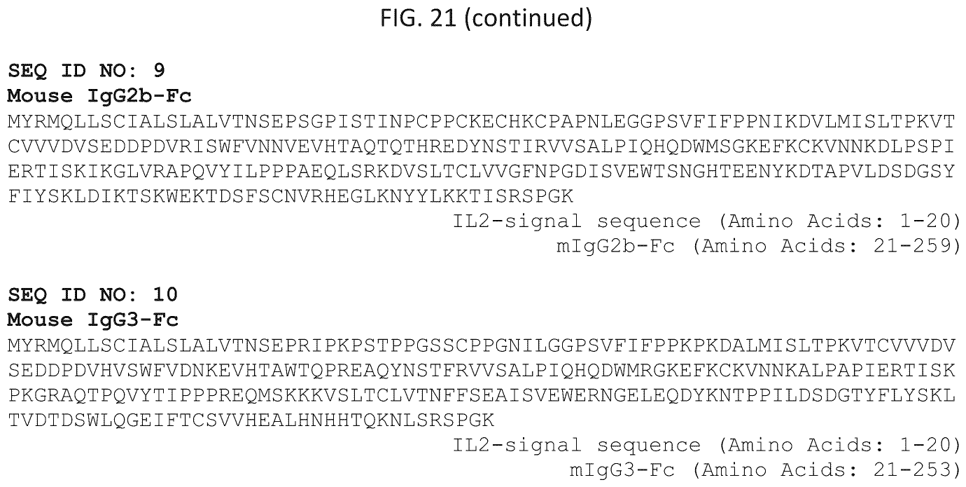

To test the in vivo activity, mice were administered with K/B×N sera and then high dose of IVIG, the original B4ST6^{Fe}, heterodimeric B4ST6Fc^{Kln} (“knobs-into-holes” B4ST6^{Fc}), and B4ST6Fc^{G3} (which has an IgG3 Fc domain without KIH mutations) were administered to the mice. Paw swelling was monitored over the next several days. As shown in FIG. 19, in the PBS-treated controls (circle), K/B×N sera induced robust inflammation in the paw. In contrast, high dose of IVIG (square), B4ST6^{Fe} (solid triangles), heterodimeric B4ST6Fc^{Kln}, (dotted line and diamonds), and B4ST6Fc^{G3} (inverted triangles) all attenuated inflammation. The results indicate that the original B4ST6^{Fe}, heterodimeric B4ST6Fc^{Kln}, and B4ST6Fc^{G3} can attenuate inflammation in arthritis models, and thus can be used to treat autoimmune disorders.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Ackerman, M. E., Crispin, M., Yu, X., Baruah, K., Boesch, A. W., Harvey, D. J., Dugast, A. S., Heizen, E. L., Ercan, A., Choi, I., Streeck, H., Nigrovic, P. A., Bailey-Kellogg, C., Scanlan, C. & Alter, G. 2013. Natural variation in Fc glycosylation of HIV-specific antibodies impacts antiviral activity. J Clin Invest, 123, 2183-92.

Albert, H., Collin, M., Dudziak, D., Ravetch, J. V. & Nimmerjahn, F. 2008. In vivo enzymatic modulation of IgG glycosylation inhibits autoimmune disease in an IgG subclass-dependent manner. Proc Natl Acad Sci USA, 105, 15005-9.

Allhorn, M., Briceno, J. G., Baudino, L., Lood, C., Olsson, M. L., Izui, S. & Collin, M. 2010. The IgG-specific endoglycosidase EndoS inhibits both cellular and complement-mediated autoimmune hemolysis. Blood, 115, 5080-8.
Anthony, R. M., Kobayashi, T., Wermeling, F. & Ravetch, J. V. 2011. Intravenous gammaglobulin suppresses inflammation through a novel T(H)2 pathway. Nature, 475, 110-3.
Anthony, R. M., Nimmerjahn, F., Ashline, D. J., Reinhold, V. N., Paulson, J. C. & Ravetch, J. V. 2008a. Recapitulation of IVIG anti-inflammatory activity with a recombinant IgG Fc. Science, 320, 373-6.
Anthony, R. M., Wermeling, F., Karlsson, M. C. & Ravetch, J. V. 2008b. Identification of a receptor required for the anti-inflammatory activity of IVIG. Proc Natl Acad Sci USA, 105, 19571-8.
Arnold, J. N., Wormald, M. R., Sim, R. B., Rudd, P. M. & Dwek, R. A. 2007. The impact of glycosylation on the biological function and structure of human immunoglobulins. Annu Rev Immunol, 25, 21-50.
Bayry, J., Bansal, K., Kazatchkine, M. D. & Kaveri, S. V. 2009. DC-SIGN and alpha2,6-sialylated IgG Fc interaction is dispensable for the anti-inflammatory activity of IVIg on human dendritic cells. Proc Natl Acad Sci USA, 106, E24; author reply E25.
Beck, A., Wurch, T., Bailly, C. & Corvaia, N. 2010. Strategies and challenges for the next generation of therapeutic antibodies. Nat Rev Immunol, 10, 345-52.
Benkhoucha, M., Molnarfi, N., Santiago-Raber, M. L., Weber, M. S., Merkler, D., Collin, M. & Lalive, P. H. 2012. IgG glycan hydrolysis by EndoS inhibits experimental autoimmune encephalomyelitis. J Neuroinflammation, 9, 209.
Boilard, E., Nigrovic, P. A., Larabee, K., Watts, G. F., Coblyn, J. S., Weinblatt, M. E., Massarotti, E. M., Remold-O'donnell, E., Famdale, R. W., Ware, J. & Lee, D. M. 2010. Platelets amplify inflammation in arthritis via collagen-dependent microparticle production. Science, 327, 580-3.
Bruhns, P., Samuelsson, A., Pollard, J. W. & Ravetch, J. V. 2003. Colony-stimulating factor-1-dependent macrophages are responsible for IVIG protection in antibody-induced autoimmune disease. Immunity, 18, 573-81.
Chung, A. W., Ghebremichael, M., Robinson, H., Brown, E., Choi, I., Lane, S., Dugast, A. S., Schoen, M. K., Rolland, M., Suscovich, T. J., Mahan, A. E., Liao, L., Streeck, H., Andrews, C., Rerks-Ngarm, S., Nitayaphan, S., De Souza, M. S., Kaewkungwal, J., Pitisuttithum, P., Francis, D., Michael, N. L., Kim, J. H., Bailey-Kellogg, C., Ackerman, M. E. & Alter, G. 2014. Polyfunctional Fc-effector profiles mediated by IgG subclass selection distinguish RV 144 and VAX003 vaccines. Sci Transl Med, 6, 228ra38.
Clynes, R. 2007. Protective mechanisms of IVIG. Curr Opin Immunol, 19, 646-51.
Collin, M. & Olsen, A. 2001. EndoS, a novel secreted protein from Streptococcus pyogenes with endoglycosidase activity on human IgG. EMBO J, 20, 3046-55.
Davies, J., Jiang, L., Pan, L. Z., Labarre, M. J., Anderson, D. & Reff, M. 2001. Expression of GnTIII in a recombinant anti-CD20 CHO production cell line: Expression of antibodies with altered glycoforms leads to an increase in ADCC through higher affinity for FC gamma RIIL Biotechnol Bioeng, 74, 288-94.
Debre, M., Bonnet, M. C., Fridman, W. H., Carosella, E., Philippe, N., Reinert, P., Vilmer, E., Kaplan, C., Teillaud, J. L. & Griscelli, C. 1993. Infusion of Fc gamma fragments for treatment of children with acute immune thrombocytopenic purpura. Lancet, 342, 945-9.
Devi, S., Kuligowski, M. P., Kwan, R. Y., Westein, E., Jackson, S. P., Kitching, A. R. & Hickey, M. J. 2010. Platelet recruitment to the inflamed glomerulus occurs via an alphaIIbbeta3/GPVI-dependent pathway. Am J Pathol, 177, 1131-42.
Ferrara, C., Grau, S., Jager, C., Sondermann, P., Brunker, P., Waldhauer, I., Hennig, M., Ruf, A., Rufer, A. C., Stihle, M., Umana, P. & Benz, J. 2011. Unique carbohydrate-carbohydrate interactions are required for high affinity binding between Fc{gamma}RIII and antibodies lacking core fucose. Proc Natl Acad Sci USA, 108, 12669-74
Fiebiger, B. M., Maamary, J., Pincetic, A. & Ravetch, J. V. 2015. Protection in antibody- and T cell-mediated autoimmune diseases by antiinflammatory IgG Fcs requires type II FcRs. Proc Natl Acad Sci USA, 112, E2385-94.
Franklin, E. C. 1975. Structure and function of immunoglobulins. Acta Endocrinol Suppl (Copenh), 194, 77-95.
Huber, R., Deisenhofer, J., Colman, P. M., Matsushima, M. & Palm, W. 1976. Crystallographic structure studies of an IgG molecule and an Fc fragment. Nature, 264, 415-20.
Imbach, P., Barandun, S., D'apuzzo, V., Baumgartner, C., Hirt, A., Morell, A., Rossi, E., Schoni, M., Vest, M. & Wagner, H. P. 1981. High-dose intravenous gammaglobulin for idiopathic thrombocytopenic purpura in childhood. Lancet, 1, 1228-31.
Jefferis, R. 2005. Glycosylation of recombinant antibody therapeutics. Biotechnol Prog, 21, 11-6.
Jefferis, R. 2009a. Glycosylation as a strategy to improve antibody-based therapeutics. Nat Rev Drug Discov, 8, 226-34.
Jefferis, R. 2009b. Glycosylation of antibody therapeutics: optimisation for purpose. Methods Mol Biol, 483, 223-38.
Jones, M. B., Nasirikenari, M., Lugade, A. A., Thanavala, Y. & Lau, J. T. 2012. Anti-inflammatory IgG production requires functional P1 promoter in beta-galactoside alpha2,6-sialyltransferase 1 (ST6Gal-1) gene. J Biol Chem, 287, 15365-70.
Jones, M. B., Oswald, D. M., Joshi, S., Whiteheart, S. W., Orlando, R. & Cobb, B. A. 2016. B-cell-independent sialylation of IgG. Proc Natl Acad Sci USA, 113, 7207-12.
Kalcheva, I., Elliott, R. W., Dalziel, M. & Lau, J. T. 1997. The gene encoding beta-galactoside alpha2,6-sialyltransferase maps to mouse chromosome 16. Mamm Genome, 8, 619-20.
Kaneko, Y., Nimmerjahn, F., Madaio, M. P. & Ravetch, J. V. 2006a. Pathology and protection in nephrotoxic nephritis is determined by selective engagement of specific Fc receptors. J Exp Med, 203, 789-97.
Kaneko, Y., Nimmerjahn, F. & Ravetch, J. V. 2006b. Anti-inflammatory activity of immunoglobulin G resulting from Fc sialylation. Science, 313, 670-3.
Kang, Y. S., Kim, J. Y., Bruening, S. A., Pack, M., Charalambous, A., Pritsker, A., Moran, T. M., Loeffler, J. M., Steinman, R. M. & Park, C. G. 2004. The C-type lectin SIGN-R1 mediates uptake of the capsular polysaccharide of Streptococcus pneumoniae in the marginal zone of mouse spleen. Proc Natl Acad Sci USA, 101, 215-20.
Korganow, A. S., Ji, H., Mangialaio, S., Duchatelle, V., Pelanda, R., Martin, T., Degott, C., Kikutani, H., Rajewsky, K., Pasquali, J. L., Benoist, C. & Mathis, D. 1999. From systemic T cell self-reactivity to organ-specific autoimmune disease via immunoglobulins. Immunity, 10, 451-61.
Lee, M. M., Nasirikenari, M., Manhardt, C. T., Ashline, D. J., Hanneman, A. J., Reinhold, V. N. & Lau, J. T. 2014. Platelets support extracellular sialylation by supplying the sugar donor substrate. J Biol Chem, 289, 8742-8.
Lerner, R. A., Glassock, R. J. & Dixon, F. J. 1967. The role of anti-glomerular basement membrane antibody in the pathogenesis of human glomerulonephritis. J Exp Med, 126, 989-1004.
Li, T., Dilillo, D. J., Bournazos, S., Giddens, J. P., Ravetch, J. V. & Wang, L. X. 2017. Modulating IgG effector function by Fc glycan engineering. Proc Natl Acad Sci USA, 114, 3485-3490.
Lu, L. L., Chung, A. W., Rosebrock, T. R., Ghebremichael, M., Yu, W. H., Grace, P. S., Schoen, M. K., Tafesse, F., Martin, C., Leung, V., Mahan, A. E., Sips, M., Kumar, M. P., Tedesco, J., Robinson, H., Tkachenko, E., Draghi, M., Freedberg, K. J., Streeck, H., Suscovich, T. J., Lauffenburger, D. A., Restrepo, B. I., Day, C., Fortune, S. M. & Alter, G. 2016. A Functional Role for Antibodies in Tuberculosis. Cell, 167, 433-443 e14.
Meng, L., Forouhar, F., Thieker, D., Gao, Z., Ramiah, A., Moniz, H., Xiang, Y., Seetharaman, J., Milaninia, S., Su, M., Bridger, R., Veillon, L., Azadi, P., Komhaber, G., Wells, L., Montelione, G. T., Woods, R. J., Tong, L. & Moremen, K. W. 2013. Enzymatic basis for N-glycan sialylation: structure of rat alpha2,6-sialyltransferase (ST6GAL1) reveals conserved and unique features for glycan sialylation. J Biol Chem, 288, 34680-98.
Natsume, A., Wakitani, M., Yamane-Ohnuki, N., Shoji-Hosaka, E., Niwa, R., Uchida, K., Satoh, M. & Shitara, K. 2005. Fucose removal from complex-type oligosaccharide enhances the antibody-dependent cellular cytotoxicity of single-gene-encoded antibody comprising a single-chain antibody linked the antibody constant region. J Immunol Methods, 306, 93-103.
Negi, V. S., Elluru, S., Siberil, S., Graff-Dubois, S., Mouthon, L., Kazatchkine, M. D., Lacroix-Desmazes, S., Bayry, J. & Kaveri, S. V. 2007. Intravenous immunoglobulin: an update on the clinical use and mechanisms of action. J Clin Immunol, 27, 233-45.
Nimmerjahn, F., Gordan, S. & Lux, A. 2015. FcgammaR dependent mechanisms of cytotoxic, agonistic, and neutralizing antibody activities. Trends Immunol, 36, 325-36.
Nimmerjahn, F. & Ravetch, J. V. 2008a. Anti-inflammatory actions of intravenous immunoglobulin. Annu Rev Immunol, 26, 513-33.
Nimmerjahn, F. & Ravetch, J. V. 2008b. Fcgamma receptors as regulators of immune responses. Nat Rev Immunol, 8, 34-47.
Ohmi, Y., Ise, W., Harazono, A., Takakura, D., Fukuyama, H., Baba, Y., Narazaki, M., Shoda, H., Takahashi, N., Ohkawa, Y., Ji, S., Sugiyama, F., Fujio, K., Kumanogoh, A., Yamamoto, K., Kawasaki, N., Kurosaki, T., Takahashi, Y. & Furukawa, K. 2016. Sialylation converts arthritogenic IgG into inhibitors of collagen-induced arthritis. Nat Commun, 7, 11205.
Okazaki, A., Shoji-Hosaka, E., Nakamura, K., Wakitani, M., Uchida, K., Kakita, S., Tsumoto, K., Kumagai, I. & Shitara, K. 2004. Fucose depletion from human IgG1 oligosaccharide enhances binding enthalpy and association rate between IgG1 and FcgammaRIlla. J Mol Biol, 336, 1239-49.
Pfeifle, R., Rothe, T., Ipseiz, N., Scherer, H. U., Culemann, S., Harre, U., Ackermann, J. A., Seefried, M., Kleyer, A., Uderhardt, S., Haugg, B., Hueber, A. J., Daum, P., Heidkamp, G. F., Ge, C., Bohm, S., Lux, A., Schuh, W., Magorivska, I., Nandakumar, K. S., Lonnblom, E., Becker, C., Dudziak, D., Wuhrer, M., Rombouts, Y., Koeleman, C. A., Toes, R., Winkler, T. H., Holmdahl, R., Herrmann, M., Bluml, S., Nimmerjahn, F., Schett, G. & Kronke, G. 2017. Regulation of autoantibody activity by the IL-23-TH17 axis determines the onset of autoimmune disease. Nat Immunol, 18, 104-113.
Pincetic, A., Bournazos, S., Dilillo, D. J., Maamary, J., Wang, T. T., Dahan, R., Fiebiger, B. M. & Ravetch, J. V. 2014. Type I and type II Fc receptors regulate innate and adaptive immunity. Nat Immunol, 15, 707-16.
Pucci, F., Rickelt, S., Newton, A. P., Garris, C., Nunes, E., Evavold, C., Pfirschke, C., Engblom, C., Mino-Kenudson, M., Hynes, R. O., Weissleder, R. & Pittet, M. J. 2016. PF4 Promotes Platelet Production and Lung Cancer Growth. Cell Rep, 17, 1764-1772.
Samuelsson, A., Towers, T. L. & Ravetch, J. V. 2001. Anti-inflammatory activity of IVIG mediated through the inhibitory Fc receptor. Science, 291, 484-6.
Scallon, B. J., Tam, S. H., Mccarthy, S. G., Cai, A. N. & Raju, T. S. 2007. Higher levels of sialylated Fc glycans in immunoglobulin G molecules can adversely impact functionality. Mol Immunol, 44, 1524-34.
Schlothauer, T., Herter, S., Koller, C. F., Grau-Richards, S., Steinhart, V., Spick, C., Kubbies, M., Klein, C., Umana, P. & Mossner, E. 2016. Novel human IgG1 and IgG4 Fc-engineered antibodies with completely abolished immune effector functions. Protein Eng Des Sel, 29, 457-466.
Schrijver, G., Bogman, M. J., Assmann, K. J., De Waal, R. M., Robben, H. C., Van Gasteren, H. & Koene, R. A. 1990. Anti-GBM nephritis in the mouse: role of granulocytes in the heterologous phase. Kidney Int, 38, 86-95.
Schwab, I., Biburger, M., Kronke, G., Schett, G. & Nimmerjahn, F. 2012. IVIg-mediated amelioration of ITP in mice is dependent on sialic acid and SIGNR1. Eur J Immunol, 42, 826-30.
Schwab, I., Mihai, S., Seeling, M., Kasperkiewicz, M., Ludwig, R. J. & Nimmerjahn, F. 2014. Broad requirement for terminal sialic acid residues and FcgammaRIIB for the preventive and therapeutic activity of intravenous immunoglobulins in vivo. Eur J Immunol, 44, 1444-53.
Schwab, L & Nimmerjahn, F. 2013. Intravenous immunoglobulin therapy: how does IgG modulate the immune system? Nat Rev Immunol, 13, 176-89.
Shields, R. L., Lai, J., Keck, R., O'connell, L. Y., Hong, K., Meng, Y. G., Weikert, S. H. & Presta, L. G. 2002. Lack of fucose on human IgG1 N-linked oligosaccharide improves binding to human Fcgamma RIII and antibody-dependent cellular toxicity. J Biol Chem, 277, 26733-40.
Stadlmann, J., Weber, A., Pabst, M., Anderle, H., Kunert, R., Ehrlich, H. J., Peter Schwarz, H. & Altmann, F. 2009. A close look at human IgG sialylation and subclass distribution after lectin fractionation. Proteomics, 9, 4143-53.
Sugimoto, I., Futakawa, S., Oka, R., Ogawa, K., Marth, J. D., Miyoshi, E., Taniguchi, N., Hashimoto, Y. & Kitazume, S. 2007. Beta-galactoside alpha2,6-sialyltransferase I cleavage by BACE1 enhances the sialylation of soluble glycoproteins. A novel regulatory mechanism for alpha2,6-sialylation. J Biol Chem, 282, 34896-903.
Tackenberg, B., Jelcic, I., Baerenwaldt, A., Oertel, W. H., Sommer, N., Nimmerjahn, F. & Lunemann, J. D. 2009. Impaired inhibitory Fcgamma receptor IIB expression on B cells in chronic inflammatory demyelinating polyneuropathy. Proc Natl Acad Sci USA, 106, 4788-92.
Tackenberg, B., Nimmerjahn, F. & Lunemann, J. D. 2010. Mechanisms of IVIG efficacy in chronic inflammatory demyelinating polyneuropathy. J Clin Immunol, 30 Suppl 1, S65-9.
Tan, M., Yan, H. B., Li, J. N., Li, W. K., Fu, Y. Y., Chen, W. & Zhou, Z. 2016. Thrombin Stimulated Platelet-Derived Exosomes Inhibit Platelet-Derived Growth Factor Receptor-Beta Expression in Vascular Smooth Muscle Cells. Cell Physiol Biochem, 38, 2348-65.
Wang, T. T., Maamary, J., Tan, G. S., Boumazos, S., Davis, C. W., Krammer, F., Schlesinger, S. J., Palese, P., Ahmed, R. & Ravetch, J. V. 2015. Anti-HA Glycoforms Drive B Cell Affinity Selection and Determine Influenza Vaccine Efficacy. Cell, 162, 160-9.
Wang, T. T., Sewatanon, J., Memoli, M. J., Wrammert, J., Boumazos, S., Bhaumik, S. K., Pinsky, B. A., Chokephaibulkit, K., Onlamoon, N., Pattanapanyasat, K., Taubenberger, J. K., Ahmed, R. & Ravetch, J. V. 2017. IgG antibodies to dengue enhanced for FcgammaRIIIA binding determine disease severity. Science, 355, 395-398.
Wang, X., Vertino, A., Eddy, R. L., Byers, M. G., Jani-Sait, S. N., Shows, T. B. & Lau, J. T. 1993. Chromosome mapping and organization of the human beta-galactoside alpha 2,6-sialyltransferase gene. Differential and cell-type specific usage of upstream exon sequences in B-lymphoblastoid cells. J Biol Chem, 268, 4355-61.
Washburn, N., Schwab, I., Ortiz, D., Bhatnagar, N., Lansing, J. C., Medeiros, A., Tyler, S., Mekala, D., Cochran, E., Sarvaiya, H., Garofalo, K., Meccariello, R., Meador, J. W., 3rd, Rutitzky, L., Schultes, B. C., Ling, L., Avery, W., Nimmerjahn, F., Manning, A. M., Kaundinya, G. V. & Bosques, C. J. 2015. Controlled tetra-Fc sialylation of IVIg results in a drug candidate with consistent enhanced anti-inflammatory activity. Proc Natl Acad Sci USA, 112, E1297-306.
Woodard-Grice, A. V., Mcbrayer, A. C., Wakefield, J. K., Zhuo, Y. & Bellis, S. L. 2008. Proteolytic shedding of ST6Gal-I by BACE1 regulates the glycosylation and function of alpha4beta1 integrins. J Biol Chem, 283, 26364-73.
Xiao, H., Woods, E. C., Vukojicic, P. & Bertozzi, C. R. 2016. Precision glycocalyx editing as a strategy for cancer inimunotherapy. Proc Natl Acad Sci USA, 113, 10304-9.
Yang, R., Otten, M. A., Hellmark, T., Collin, M., Bjorck, L., Zhao, M. H., Daha, M. R. & Segelmark, M. 2010. Successful treatment of experimental glomerulonephritis with IdeS and EndoS, IgG-degrading streptococcal enzymes. Nephrol Dial Transplant, 25, 2479-86.
Youings, A., Chang, S. C., Dwek, R. A. & Scragg, I. G. 1996. Site-specific glycosylation of human immunoglobulin G is altered in four rheumatoid arthritis patients. Biochem J, 314 (Pt 2), 621-30.
Zhang, G., Massaad, C. A., Gao, T., Pillai, L., Bogdanova, N., Ghauri, S. & Sheikh, K. A. 2016. Sialylated intravenous immunoglobulin suppress anti-ganglioside antibody mediated nerve injury. Exp Neurol, 282, 49-55.

1. A fusion polypeptide comprising:
an antibody heavy chain CH2 region;
an antibody heavy chain CH3 region; and
a catalytic domain of a sialyltransferase, wherein the catalytic domain of the sialyltransferase catalyzes sialylation of a glycoprotein.

2. The fusion polypeptide of claim 1, wherein the sialyltransferase is beta-galactoside alpha-2,6 sialyltransferase 1.

3. The fusion polypeptide of claim 1, wherein the sialyltransferase is a human sialyltransferase.

4. The fusion polypeptide of claim 1, wherein the antibody heavy chain CH2 region comprises a human IgG heavy chain CH2 region.

5. The fusion polypeptide of claim 1, wherein the antibody heavy chain CH3 region is a human IgG heavy chain CH3 region.

6. A fusion polypeptide comprising:
an antibody heavy chain CH2 region;
an antibody heavy chain CH3 region; and
a catalytic domain of galactosyltransferase, wherein the catalytic domain of galactosyltransferase catalyzes galactosylation of a glycoprotein.

7. The fusion polypeptide of claim 6, wherein the galactosyltransferase is beta-1,4 galactosyltransferase 1.

8. The fusion polypeptide of claim 6, wherein the galactosyltransferase is a human galactosyltransferase.

9. The fusion polypeptide of claim 6, wherein the antibody heavy chain CH2 region is a human IgG heavy chain CH2 region.

10. The fusion polypeptide of claim 6, wherein the antibody heavy chain CH3 region is a human IgG heavy chain CH3 region.

11. A polynucleotide encoding the fusion polypeptide of claim 1.

12. A vector comprising a polynucleotide encoding the fusion polypeptide of claim 1.

13. A cell comprising the vector of claim 12.

14. A heteromultimer comprising
a first fusion polypeptide comprising an antibody heavy chain CH2 region, an antibody heavy chain CH3 region, and a catalytic domain of sialyltransferase, wherein the catalytic domain of sialyltransferase catalyzes sialylation of a glycoprotein; and
a second fusion polypeptide comprising an antibody heavy chain CH2 region, an antibody heavy chain CH3 region, and a catalytic domain of galactosyltransferase, wherein the catalytic domain of galactosyltransferase catalyzes galactosylation of a glycoprotein.

15. The heteromultimer of claim 14, wherein the heteromultimer is a heterodimer, and the first fusion polypeptide associates with the second fusion polypeptide, thereby forming the heterodimer.

16. The heteromultimer of claim 14, wherein the sialyltransferase is beta-galactoside alpha-2,6 sialyltransferase 1.

17. The heteromultimer of claim 14, wherein the sialyltransferase is a human sialyltransferase.

18. The heteromultimer of claim 14, wherein the galactosyltransferase is beta-1,4-galactosyltransferase 1.

19. The heteromultimer of claim 14, wherein the galactosyltransferase is a human galactosyltransferase.

20. A method of treating a subject having an IgG-mediated disorder, the method comprising:
administering to the subject an effective amount of a composition comprising the heteromultimer of claim 14.

21-51. (canceled)
